SEQUENCE LISTING (22 sequences)

SEQ ID NO: 1 - protein, 509 aa, human herpesvirus 1
MAPGRVGLAVVLWGLLWLGAGVAGGSETASTGPTITAGAVTNASEAPTSGSPGSAASPEVTPTSTPNPNNVTQNKTTPTEPASPPTTPKPTSTPKSPPTSTPDPKPKNNTTPAKSGRPTKPPGPVWCDRRDPLARYGSRVQIRCRFRNSTRMEFRLQIWRYSMGPSPPIAPAPDLEEVLTNITAPPGGLLVYDSAPNLTDPHVLWAEGAGPGADPPLYSVTGPLPTQRLIIGEVTPATQGMYYLAWGRMDSPHEYGTWVRVRMFRPPSLTLQPHAVMEGQPFKATCTAAAYYPRNPVEFDWFEDDRQVFNPGQIDTQTHEHPDGFTTVSTVTSEAVGGQVPPRTFTCQMTWHRDSVTFSRRNATGLALVLPRPTMEFGVRHVVCTAGCVPEGVTFAWFLGDDPSPAAKSAVTAQESCDHPGLATVRSTLPISYDYSEYICRLTGYPAGIPVLEHHGSHQPPPRDPTERQVIEAIEWVGIGIGVLAAGVLVVTAIVYVVRTSQSRQRHRR

SEQ ID NO: 2 - protein, 478 aa, human herpesvirus 2
MALGRVGLAVGLWGLLWVGVVVVLANASPGRTVGPRGNASNAAPSASPRNASAPRTTPTPPQPRKATKSKASTAKPAPPPKTGPPKTSSEPVRCNRHDPLARYGSRVQIRCRFPNSTRTEFRLQIWRYATATDAEIGTAPSLEEVMVNVSAPPGGQLVYDSAPNRTDPHVIWAEGAGPGASPRLYSVVGPLGRQRLIIEELTLETQGMYYWVWGRTDRPSAYGTWVRVRVFRPPSLTIHPHAVLEGQPFKATCTAATYYPGNRAEFVWFEDGRRVFDPAQIHTQTQENPDGFSTVSTVTSAAVGGQGPPRTFTCQLTWHRDSVSFSRRNASGTASVLPRPTITMEFTGDHAVCTAGCVPEGVTFAWFLGDDSSPAEKVAVASQTSCGRPGTATIRSTLPVSYEQTEYICRLAGYPDGIPVLEHHGSHQPPPRDPTERQVIRAVEGAGIGVAVLVAVVLAGTAVVYLTHASSVRYRRLR

SEQ ID NO: 3 - protein, 394 aa, human herpesvirus 1
MGGAAARLGAVILFVVIVGLHGVRGKYALADASLKLADPNRFRRKDLPVLDQLTDPPGVRRVYHIQAGLPDPFQPPSLPITVYYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRFIPENQRTVAVYSLKIAGWHGPKAPYTSTLLPPELSETPNATQPELAPEAPEDSALLEDPVGTVAPQIPPNWHIPSIQDAATPYHPPATPNNMGLIAGAVGGSLLAALVICGIVYWMRRRTQKAPKRIRLPHIREDDQPSSHQPLFY

SEQ ID NO: 4 - protein, 391 aa, human herpesvirus 2
MGRLTSGVGTAALLVVAVGLRVVCAKYALADPSLKMADPNRFRGKNLPVLDQLTDPPGVKRVYHIQPSLEDPFQPPSIPITVYYAVLERACRSVLLHAPSEAPQIVRGASDEARKHTYNLTIAWYRMGDNCAIPITVMEYTECPYNKSLGVCPIRTQPRWSYYDSFSAVSEDNLGFLMHAPAFETAGTYLRLVKINDWTEQFILEHRARASCKYALPLRIPPAACLTSKAYQQGVTVDSIGMLPRFIPENQRTVALYSLKIAGWHGPKPPYTSTLLPPELSDTTNATQPELVPEDPEDSALLEDPAGTVSSQIPPNWHIPSIQDVAPHHAPAAPSNPGLIIGALAGSTLAVLVIGGIAFWVRRRAQMAPKRLRLPHIRDDDAPPSHQPLFY

SEQ ID NO: 5 - protein, 552 aa, human herpesvirus 1
MDRGAVVGFLLGVCVVSCLAGTPKTSWRRVSVGEDVSLLPAPGPTGRGPTQKLLWAVEPLDGCGPLHPSWVSLMPPKQVPETVVDAACMRAPVPLAMAYAPPAPSATGGLRTDFVWQERAAVVNRSLVIYGVRETDSGLYTLSVGDIKDPARQVASVVLVVQPAPVPTPPPTPADYDEDDNDEGEGEDESLAGTPASGTPRLPPSPAPPRSWPSAPEVSHVRGVTVRMETPEAILFSPGEAFSTNVSIHAIAHDDQTYTMDVVWLRFDVPTSCAEMRIYESCLYHPQLPECLSPADAPCAASTWTSRLAVRSYAGCSRTNPPPRCSAEAHMEPFPGLAWQAASVNLEFRDASPQHSGLYLCVVYVNDHIHAWGHITINTAAQYRNAVVEQPLPQRGADLAEPTHPHVGAPPHAPPTHGALRLGAVMGAALLLSALGLSVWACMTCWRRRAWRAVKSRASGKGPTYIRVADSELYADWSSDSEGERDQVPWLAPPERPDSPSTNGSGFEILSPTAPSVYPRSDGHQSRRQLTTFGSGRPDRRYSQASDSSVFW

SEQ ID NO: 6 - protein, 543 aa, human herpesvirus 2
MARGAGLVFFVGVWVVSCLAAAPRTSWKRVTSGEDVVLLPAPAERTRAHKLLWAAEPLDACGPLRPSWVALWPPRRVLETVVDAACMRAPEPLAIAYSPPFPAGDEGLYSELAWRDRVAVVNESLVIYGALETDSGLYTLSVVGLSDEARQVASVVLVVEPAPVPTPTPDDYDEEDDAGVTNARRSAFPPQPPPRRPPVAPPTHPRVIPEVSHVRGVTVHMETLEAILFAPGETFGTNVSIHAIAHDDGPYAMDVVWMRFDVPSSCADMRIYEACLYHPQLPECLSPADAPCAVSSWAYRLAVRSYAGCSRTTPPPRCFAEARMEPVPGLAWLASTVNLEFQHASPQHAGLYLCVVYVDDHIHAWGHMTISTAAQYRNAVVEQHLPQRQPEPVEPTRPHVRAPHPAPSARGPLRLGAVLGAALLLAALGLSAWACMTCWRRRSWRAVKSRASATGPTYRVADSELYADWSSDSEGERDGSLWQDPPERPDSPSTNGSGFEILSPTAPSVYPHSEGRKSRRPLTTFGSGSPGRRHSQASYPSVL

SEQ ID NO: 7 - protein, 904 aa, human herpesvirus 1
MHQGAPSWGRRWFVVWALLGLTLGVLVASAAPTSPGTPGVAAATQAANGGPATPAPPPLGAAPTGDPKPKKNKKPKNPTPPRPAGDNATVAAGHATLREHLRDIKAENTDANFYVCPPPTGATVVQFEQPRRCPTRPEGQNYTEGIAVVFKENIAPYKFKATMYYKDVTVSQVWFGHRYSQFMGIFEDRAPVPFEEVIDKINAKGVCRSTAKYVRNNLETTAFHRDDHETDMELKPANAATRTSRGWHTTDLKYNPSRVEAFHRYGTTVNCIVEEVDARSVYPYDEFVLATGDFVYMSPFYGYREGSHTEHTTYAADRFKQVDGFYARDLTTKARATAPTTRNLLTTPKFTVAWDWVPKRPSVCTMTKWQEVDEMLRSEYGGSFRFSSDAISTTFTTNLTEYPLSRVDLGDCIGKDARDAMDRIFARRYNATHIKVGQPQYYQANGGFLIAYQPLLSNTLAELYVREHLREQSRKPPNPTPPPPGASANASVERIKTTSSIEFARLQFTYNHIQRHVNDMLGRVAIAWCELQNHELTLWNEARKLNPNAIASVTVGRRVSARMLGDVMAVSTCVPVAADNVIVQNSMRISSRPGACYSRPLVSFRYEDQGPLVEGQLGENNELRLTRDAIEPCTVGHRRYFTFGGGYVYFEEYAYSHQLSRADITTVSTFIDLNITMLEDHEFVPLEVYTRHEIKDSGLLDYTEVQRRNQLHDLRFADIDTVIHADANAAMFAGLGAFFEGMGDLGRAVGKVVMGIVGGVVSAVSGVSSFMSNPFGALAVGLLVLAGLAAAFFAFRYVMRLQSNPMKALYPLTTKELKNPTNPDASGEGEEGGDFDEAKLAEAREMIRYMALVSAMERTEHKAKKKGTSALLSAKVTDMVMRKRRNTNYTQVPNKDGDADEDDL

SEQ ID NO: 8 - protein, 904 aa, human herpesvirus 2
MRGGGLICALVVGALVAAVASAAPAAPAAPRASGGVAATVAANGGPASRPPPVPSPATTKARKRKTKKPPKRPEATPPPDANATVAAGHATLRAHLREIKVENADAQFYVCPPPTGATVVQFEQPRRCPTRPEGQNYTEGIAVVFKENIAPYKFKATMYYKDVTVSQVWFGHRYSQFMGIFEDRAPVPFEEVIDKINTKGVCRSTAKYVRNNMETTAFHRDDHETDMELKPAKVATRTSRGWHTTDLKYNPSRVEAFHRYGTTVNCIVEEVDARSVYPYDEFVLATGDFVYMSPFYGYREGSHTEHTSYAADRFKQVDGFYARDLTTKARATSPTTRNLLTTPKFTVAWDWVPKRPAVCTMTKWQEVDEMLRAEYGGSFRFSSDAISTTFTTNLTEYSLSRVDLGDCIGRDAREAIDRMFARKYNATHIKVGQPQYYLATGGFLIAYQPLLSNTLAELYVREYMREQDRKPRNATPAPLREAPSANASVERIKTTSSIEFARLQFTYNHIQRHVNDMLGRIAVAWCELQNHELTLWNEARKLNPNAIASATVGRRVSARMLGDVMAVSTCVPVAPDNVIVQNSMRVSSRPGTCYSRPLVSFRYEDQGPLIEGQLGENNELRLTRDALEPCTVGHRRYFIFGGGYVYFEEYAYSHQLSRADVTTVSTFIDLNITMLEDHEFVPLEVYTRHEIKDSGLLDYTEVQRRNQLHDLRFADIDTVIRADANAAMFAGLCAFFEGMGDLGRAVGKVVMGVVGGVVSAVSGVSSFMSNPFGALAVGLLVLAGLVAAFFAFRYVLQLQRNPMKALYPLTTKELKTSDPGGVGGEGEEGAEGGGFDEAKLAEAREMIRYMALVSAMERTEHKARKKGTSALLSSKVTNMVLRKRNKARYSPLHNEDEAGDEDEL

SEQ ID NO: 9 - DNA, 1041 bp, Artificial Sequence
  Codon optimized HSV-2 gD fragment 26-339 with signal sequence and His tag
atgagatttccttcaatttttactgctgttttattcgcagcatcctccgcattagctaagtacgctttggctgacccatctttgaagatggctgacccaaacagattcagaggaaagaacttgccagttttggaccaattgactgacccaccaggtgttaagagagtttaccacatccaaccatctttggaggatccattccaaccaccatccatcccaatcactgtttactacgctgttttggagagagcttgtagatccgttttgttgcacgctccatctgaagctccacagattgttagaggagcttctgacgaagctagaaagcacacttacaacttgactatcgcttggtacagaatgggtgacaactgtgctatccctatcactgttatggaatacactgagtgtccatacaacaagtccttgggagtttgtccaatcagaactcagccaagatggtcctactacgattccttctctgctgtttccgaggataacttgggattcttgatgcacgctccagcttttgaaactgctggtacttacttgagattggttaagatcaacgactggactgagatcactcagttcatcttggagcacagagctagagcttcatgtaagtatgctttgccattgagaatcccacctgctgcttgtttgacttccaaggcttaccaacagggtgttactgttgattccatcggaatgttgccaagattcatcccagagaaccagagaactgttgctttgtactccttgaagatcgctggatggcatggtccaaagccaccatacacttccactttgttgccaccagaattgtccgacactacaaacgctactcagccagaattggttccagaagatccagaggattccgctttgttggaagatccagctggtactgtttcctcccaaatcccaccaaactggcacattccatccattcaagacgttgctccacatcatgctccagctgctccatctaatccaggtggtggtcatcatcaccatcaccaccaccatcactaatag

SEQ ID NO: 10 - DNA, 942 bp, Artificial Sequence
  Codon optimized HSV-2 gD fragment 26-339
aagtacgctttggctgacccatctttgaagatggctgacccaaacagattcagaggaaagaacttgccagttttggaccaattgactgacccaccaggtgttaagagagtttaccacatccaaccatctttggaggatccattccaaccaccatccatcccaatcactgtttactacgctgttttggagagagcttgtagatccgttttgttgcacgctccatctgaagctccacagattgttagaggagcttctgacgaagctagaaagcacacttacaacttgactatcgcttggtacagaatgggtgacaactgtgctatccctatcactgttatggaatacactgagtgtccatacaacaagtccttgggagtttgtccaatcagaactcagccaagatggtcctactacgattccttctctgctgtttccgaggataacttgggattcttgatgcacgctccagcttttgaaactgctggtacttacttgagattggttaagatcaacgactggactgagatcactcagttcatcttggagcacagagctagagcttcatgtaagtatgctttgccattgagaatcccacctgctgcttgtttgacttccaaggcttaccaacagggtgttactgttgattccatcggaatgttgccaagattcatcccagagaaccagagaactgttgctttgtactccttgaagatcgctggatggcatggtccaaagccaccatacacttccactttgttgccaccagaattgtccgacactacaaacgctactcagccagaattggttccagaagatccagaggattccgctttgttggaagatccagctggtactgtttcctcccaaatcccaccaaactggcacattccatccattcaagacgttgctccacatcatgctccagctgctccatctaatcca

SEQ ID NO: 11 - DNA, 954 bp, Artificial Sequence
  Codon optimized HSV-2 gD 26-310 fragment with signal sequence and His tag
atgagatttccttcaatttttactgctgttttattcgcagcatcctccgcattagctaagtacgctttggctgacccatctttgaagatggctgacccaaacagattcagaggaaagaacttgccagttttggaccaattgactgacccaccaggtgttaagagagtttaccacatccaaccatctttggaggatccattccaaccaccatccatcccaatcactgtttactacgctgttttggagagagcttgtagatccgttttgttgcacgctccatctgaagctccacagattgttagaggagcttctgacgaagctagaaagcacacttacaacttgactatcgcttggtacagaatgggtgacaactgtgctatccctatcactgttatggaatacactgagtgtccatacaacaagtccttgggagtttgtccaatcagaactcagccaagatggtcctactacgattccttctctgctgtttccgaggataacttgggattcttgatgcacgctccagcttttgaaactgctggtacttacttgagattggttaagatcaacgactggactgagatcactcagttcatcttggagcacagagctagagcttcatgtaagtatgctttgccattgagaatcccacctgctgcttgtttgacttccaaggcttaccaacagggtgttactgttgattccatcggaatgttgccaagattcatcccagagaaccagagaactgttgctttgtactccttgaagatcgctggatggcatggtccaaagccaccatacacttccactttgttgccaccagaattgtccgacactacaaacgctactcagccagaattggttccagaagatccagaggattccgctttgttggaagatccagctggtactggtggtggtcatcatcaccatcaccaccaccatcactaatag

SEQ ID NO: 12 - DNA, 855 bp, Artificial Sequence
  Codon optimized HSV-2 gD 26-310 fragment
aagtacgctttggctgacccatctttgaagatggctgacccaaacagattcagaggaaagaacttgccagttttggaccaattgactgacccaccaggtgttaagagagtttaccacatccaaccatctttggaggatccattccaaccaccatccatcccaatcactgtttactacgctgttttggagagagcttgtagatccgttttgttgcacgctccatctgaagctccacagattgttagaggagcttctgacgaagctagaaagcacacttacaacttgactatcgcttggtacagaatgggtgacaactgtgctatccctatcactgttatggaatacactgagtgtccatacaacaagtccttgggagtttgtccaatcagaactcagccaagatggtcctactacgattccttctctgctgtttccgaggataacttgggattcttgatgcacgctccagcttttgaaactgctggtacttacttgagattggttaagatcaacgactggactgagatcactcagttcatcttggagcacagagctagagcttcatgtaagtatgctttgccattgagaatcccacctgctgcttgtttgacttccaaggcttaccaacagggtgttactgttgattccatcggaatgttgccaagattcatcccagagaaccagagaactgttgctttgtactccttgaagatcgctggatggcatggtccaaagccaccatacacttccactttgttgccaccagaattgtccgacactacaaacgctactcagccagaattggttccagaagatccagaggattccgctttgttggaagatccagctggtact

SEQ ID NO: 13 - DNA, 1590 bp, Artificial Sequence
  Codon optimized HSV-2 gC full length ectodomain with signal sequence and His tag
atgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagaatgggtcatcatcatcaccatcaccaccatcacggtggtggtatcgaaggtagattggctaatgcttctccaggtagaactatcactgttggtccaagaggtaacgcttctaacgctgctccatctgcttctccaagaaacgcttccgctccaagaactactccaactccaccacaaccaagaaaggctactaagtccaaggcttccactgctaaaccagctccaccaccaaaaactggtccaccaaagacttcctctgagccagttagatgtaacagacacgacccattggctagatacggttccagagttcagatcagatgtagattcccaaactccactagaactgagttcagattgcagatttggagatacgctactgctactgacgctgagattggtactgctccatccttggaagaggttatggttaacgtttctgctccaccaggtggtcaattggtttacgattccgctcctaacagaactgacccacacgttatttgggctgaaggtgctggtcctggtgcttctcctagattgtactccgttgttggtccattgggtagacagagattgatcatcgaagagttgactttggagacacagggaatgtactactgggtctggggtagaactgatagaccatccgcttacggtacttgggtccgtgttagagttttcagaccaccatccttgactattcacccacacgctgttttggaaggacaaccattcaaggctacttgtactgctgctacttactacccaggtaacagagctgagttcgtctggttcgaggacggtagaagagttttcgacccagctcaaatccacactcagacacaagaaaacccagacggtttctccactgtttccacagttacttccgctgctgttggtggtcaaggtccaccaagaactttcacttgtcagttgacttggcacagagattctgtttccttctccagaagaaatgcttctggtactgcttccgttttgccaagaccaactatcactatggagttcactggtgaccatgctgtttgcactgctggttgtgttccagagggtgttactttcgcttggtttttgggtgacgattcttctccagctgagaaagttgctgttgcttcccaaacttcctgtggtagaccaggtactgctactatcagatccactttgccagtttcctacgagcaaactgagtacatctgtagattggctggttacccagacggaatcccagttttggaacaccacggttctcatcaaccaccaccaagagacccaactgagagacaggttatcagagctatcgaaggtagataataa

SEQ ID NO: 14 - DNA, 1329 bp, Artificial Sequence
  Codon optimized HSV-2 gC full length ectodomain
atgggtcatcatcatcaccatcaccaccatcacggtggtggtatcgaaggtagattggctaatgcttctccaggtagaactatcactgttggtccaagaggtaacgcttctaacgctgctccatctgcttctccaagaaacgcttccgctccaagaactactccaactccaccacaaccaagaaaggctactaagtccaaggcttccactgctaaaccagctccaccaccaaaaactggtccaccaaagacttcctctgagccagttagatgtaacagacacgacccattggctagatacggttccagagttcagatcagatgtagattcccaaactccactagaactgagttcagattgcagatttggagatacgctactgctactgacgctgagattggtactgctccatccttggaagaggttatggttaacgtttctgctccaccaggtggtcaattggtttacgattccgctcctaacagaactgacccacacgttatttgggctgaaggtgctggtcctggtgcttctcctagattgtactccgttgttggtccattgggtagacagagattgatcatcgaagagttgactttggagacacagggaatgtactactgggtctggggtagaactgatagaccatccgcttacggtacttgggtccgtgttagagttttcagaccaccatccttgactattcacccacacgctgttttggaaggacaaccattcaaggctacttgtactgctgctacttactacccaggtaacagagctgagttcgtctggttcgaggacggtagaagagttttcgacccagctcaaatccacactcagacacaagaaaacccagacggtttctccactgtttccacagttacttccgctgctgttggtggtcaaggtccaccaagaactttcacttgtcagttgacttggcacagagattctgtttccttctccagaagaaatgcttctggtactgcttccgttttgccaagaccaactatcactatggagttcactggtgaccatgctgtttgcactgctggttgtgttccagagggtgttactttcgcttggtttttgggtgacgattcttctccagctgagaaagttgctgttgcttcccaaacttcctgtggtagaccaggtactgctactatcagatccactttgccagtttcctacgagcaaactgagtacatctgtagattggctggttacccagacggaatcccagttttggaacaccacggttctcatcaaccaccaccaagagacccaactgagagacaggttatcagagctatcgaaggtaga

SEQ ID NO: 15 - DNA, 1479 bp, Artificial Sequence
  Codon optimized HSV-2 gC 75-448 fragment with signal sequence and His tag
atgagatttccttcaatttttactgctgttttattcgcagcatcctccgcattagctgctatgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagacatcatcatcaccatcaccaccatcacggtggtggtactgctaaaccagctccaccaccaaaaactggtccaccaaagacttcctctgagccagttagatgtaacagacacgacccattggctagatacggttccagagttcagatcagatgtagattcccaaactccactagaactgagttcagattgcagatttggagatacgctactgctactgacgctgagattggtactgctccatccttggaagaggttatggttaacgtttctgctccaccaggtggtcaattggtttacgattccgctcctaacagaactgacccacacgttatttgggctgaaggtgctggtcctggtgcttctcctagattgtactccgttgttggtccattgggtagacagagattgatcatcgaagagttgactttggagacacagggaatgtactactgggtctggggtagaactgatagaccatccgcttacggtacttgggtccgtgttagagttttcagaccaccatccttgactattcacccacacgctgttttggaaggacaaccattcaaggctacttgtactgctgctacttactacccaggtaacagagctgagttcgtctggttcgaggacggtagaagagttttcgacccagctcaaatccacactcagacacaagaaaacccagacggtttctccactgtttccacagttacttccgctgctgttggtggtcaaggtccaccaagaactttcacttgtcagttgacttggcacagagattctgtttccttctccagaagaaatgcttctggtactgcttccgttttgccaagaccaactatcactatggagttcactggtgaccatgctgtttgcactgctggttgtgttccagagggtgttactttcgcttggtttttgggtgacgattcttctccagctgagaaagttgctgttgcttcccaaacttcctgtggtagaccaggtactgctactatcagatccactttgccagtttcctacgagcaaactgagtacatctgtagattggctggttacccagacggaatcccagttttggaacaccacggttctcatcaaccaccaccaagagacccaactgagagacaggttatcagagctatcgaaggtagataataa

SEQ ID NO: 16 - DNA, 1122 bp, Artificial Sequence
  Codon optimized HSV-2 gC 75-448 fragment
actgctaaaccagctccaccaccaaaaactggtccaccaaagacttcctctgagccagttagatgtaacagacacgacccattggctagatacggttccagagttcagatcagatgtagattcccaaactccactagaactgagttcagattgcagatttggagatacgctactgctactgacgctgagattggtactgctccatccttggaagaggttatggttaacgtttctgctccaccaggtggtcaattggtttacgattccgctcctaacagaactgacccacacgttatttgggctgaaggtgctggtcctggtgcttctcctagattgtactccgttgttggtccattgggtagacagagattgatcatcgaagagttgactttggagacacagggaatgtactactgggtctggggtagaactgatagaccatccgcttacggtacttgggtccgtgttagagttttcagaccaccatccttgactattcacccacacgctgttttggaaggacaaccattcaaggctacttgtactgctgctacttactacccaggtaacagagctgagttcgtctggttcgaggacggtagaagagttttcgacccagctcaaatccacactcagacacaagaaaacccagacggtttctccactgtttccacagttacttccgctgctgttggtggtcaaggtccaccaagaactttcacttgtcagttgacttggcacagagattctgtttccttctccagaagaaatgcttctggtactgcttccgttttgccaagaccaactatcactatggagttcactggtgaccatgctgtttgcactgctggttgtgttccagagggtgttactttcgcttggtttttgggtgacgattcttctccagctgagaaagttgctgttgcttcccaaacttcctgtggtagaccaggtactgctactatcagatccactttgccagtttcctacgagcaaactgagtacatctgtagattggctggttacccagacggaatcccagttttggaacaccacggttctcatcaaccaccaccaagagacccaactgagagacaggttatcagagctatcgaaggtaga

SEQ ID NO: 17 - DNA, 26 bp, Artificial Sequence
  Forward primer for HSV-2 gD 26-310 fragment
gaattcgaaacgatgagatttccttc

SEQ ID NO: 18 - DNA, 79 bp, Artificial Sequence
  Reverse primer for HSV-2 gD 26-310 fragment
ggccggccctattagtgatggtggtggtgatggtgatgatgaccaccaccagtaccagctggatcttccaacaaagcgg

SEQ ID NO: 19 - DNA, 90 bp, Artificial Sequence
  Forward PCR primer for HSV-2 gC 24-448 fragment
gagtcaaaaaatgggtcatcatcatcaccatcaccaccatcacggtggtggtatcgaaggtagattggctaatgcttctccaggtagaac

SEQ ID NO: 20 - DNA, 50 bp, Artificial Sequence
  Reverse PCR primer for HSV-2 gC 24-448 fragment
ggccggcccttattaagcaccttcaacagctctgataacctgtctctcag

SEQ ID NO: 21 - DNA, 69 bp, Artificial Sequence
  Forward PCR primer for HSV-2 gC 75-448 fragment
gagtcaaaaacatcatcatcaccatcaccaccatcacggtggtggtactgctaaaccagctccaccacc

SEQ ID NO: 22 - DNA, 33 bp, Artificial Sequence
  Reverse PCR primer for HSV-2 gC 75-448 fragment
gagtcaaaaaactgctaaaccagctccaccacc

(19) United States
(12) Patent Application Publication
(10) Pub. No.: US 2012/0121630 A1
(43) Pub. Date: May 17, 2012

(54) RECOMBINANT ECTODOMAIN EXPRESSION OF HERPES SIMPLEX VIRUS GLYCOPROTEINS IN YEAST

(76) Inventors: Janine T. Bryan, Furlong, PA (US); John W. Ballet, Norristown, PA (US); Jessica A. Flynn, Lansdale, PA (US); Danilo R. Casimiro, Harleysville, PA (US); Robert C. Davidson, Enfield, NH (US); Victoria Copeland, Bradford, VT (US); Sandra E. Rios, Enfield, NH (US); Byung-Kwon Choi, Norwich, VT (US); Stefan Wildt, New York, NY (US)

(21) Appl. No.: 13/386,508

(22) PCT Filed: Jul. 21, 2010
(86) PCT No.: PCT/US10/42719

§ 371 Date: Jan. 23, 2012

Related U.S. Application Data
(60) Provisional application No. 61/228,347, filed on Jul. 24, 2009.

(51) Int. Cl. A61K 39/245; A61P 37/06; C12N 15/38; C07K 14/035; C12P 21/00

(52) U.S. Cl. 424/186.1; 530/395; 435/69.3; 536/23.72

(57) ABSTRACT

The present invention provides Herpes Simplex Virus (HSV) gD, gC, gB and/or gE recombinant glycoproteins having a particular pre-selected N-linked glycosylation pattern as the predominant N-glycoform. The present invention also provides methods of producing these recombinant glycoproteins in yeast, preferably Pichia pastoris, which may be glycoengineered to provide particular glycosylation patterns. The present invention further provides vaccines comprising gD and gC, and optionally gB and/or gE, at least one of which has a particular pre-selected N-linked glycosylation pattern as the predominant N-glycoform. The recombinant glycoproteins are produced by a method which, in one embodiment, comprises transforming a yeast of the genus Pichia with an expression vector containing a DNA encoding an HSV glycoprotein, which is under regulation of a promoter functional in a yeast of the genus Pichia, culturing the yeast in a medium, and recovering the recombinant glycoprotein from the obtained culture. DNA encoding the recombinant glycoproteins is preferably codon-optimized to achieve optimal expression in Pichia.

### CROSS-REFERENCE TO RELATED APPLICATIONS

None

### FIELD OF INVENTION

The present invention provides Herpes Simplex Virus (HSV) recombinant glycoproteins, such as gD, gC, gB and gE, having a particular pre-selected N-linked glycosylation pattern as the predominant N-glycoform. The present invention also provides methods of producing these recombinant glycoproteins in yeast, preferably Pichia pastoris, which may be glycoengineered to provide particular glycosylation patterns. The present invention further provides vaccines comprising gD and gC, and optionally gB and/or gE, at least one of which has a particular pre-selected N-linked glycosylation pattern as the predominant N-glycoform. DNA encoding the recombinant glycoprotein is preferably codon-optimized to achieve high level expression in yeast.

### BACKGROUND OF THE INVENTION

Herpes Simplex Virus type 1 (HSV-1) and Herpes Simplex Virus type 2 (HSV-2) are common human pathogens and cause a variety of clinical illnesses, including oral-facial infections, genital herpes, ocular infections, herpes encephalitis, and neonatal herpes. HSV-1 is more frequently associated with non-genital infection and is mostly acquired during childhood via nonsexual contact. In the last decade, however, it has become an important cause of genital herpes. HSV-2 is the leading cause worldwide of sexually transmitted genital ulcers, and it infects an estimated 500,000 persons annually in the United States. Infection by HSV-2 is life-long with intermittent subclinical and clinical viral reactivation and shedding from mucosal surfaces, inducing significant physical and psychosocial morbidity. In newborns and immune-compromised individuals, primary HSV-2 infections can be particularly devastating. Despite the availability of antiviral agents to treat HSV disease, genital HSV-2 infections have remained a persistent problem with a seroprevalence of approximately 17% among 14-49 year old subjects in the United States, with much greater prevalence rates in parts of South America and Africa.

Herpes Simplex Virus is also a major risk factor for Human Immunodeficiency Virus (HIV) infection. Individuals who are seropositive for HSV-2 have a 2-fold increased risk of acquiring HIV. Acquisition rates appear greatest following initial HSV-2 infection, when HSV-2 reactivation is most frequent. Treatments and vaccine strategies aimed at reducing HSV infection may reduce HIV transmission, acquisition, and disease progression.

Thus, vaccines for preventing primary infection, reducing the incidence or disease and/or preventing recurrences of HSV-1 and HSV-2, and components useful for such vaccines, are urgently needed in the art.

### SUMMARY OF THE INVENTION

The present invention provides compositions of a recombinant Herpes Simplex Virus glycoprotein selected from the group consisting of gC, gD, gB and gE, or an immunogenic fragment thereof, said composition comprising a plurality of bi-antennary N-linked glycans attached to the glycoprotein, or immunogenic fragment thereof, said plurality of N-linked glycans comprising greater than 50 mole percent of an N-linked glycan consisting of a structure selected from the group consisting of Man₅GlcNAc₂ [2.0]; Gal₄GlcNAc₂Man₃GlcNAc₂ [5.9]; NANA₂Gal₂GlcNAc₂Man₃GlcNAc₂ [6.0]; Gal₂GlcNAc₂Man₃GlcNAc₂; GlcNAc₂Man₃GlcNAc₂; Man₃GlcNAc₂; NANAGalGlcNAcMan₅GlcNAc₂; GalGlcNAcMan₅GlcNAc₂; Gal₂GlcNAcMan₅GlcNAc₂; and GlcNAcMan₅GlcNAc₂. In particular embodiments, the glycoprotein protein is gC, gD, or an immunogenic fragment thereof. In certain embodiments, the glycoprotein is a HSV-1 glycoprotein or a HSV-2 glycoprotein.

In certain embodiments, the plurality of N-linked glycans comprises greater than 50 mole percent of an N-linked glycan consisting of a structure selected from the group consisting of Man₅GlcNAc₂ [2.0]; Gal₄GlcNAc₂Man₃GlcNAc₂ [5.9]; and NANA₂Gal₂GlcNAc₂Man₃GlcNAc₂ [6.0]. In particular embodiments, the plurality of N-linked glycans comprises greater than 75 or greater than 85 mole percent of an N-linked glycan consisting of Man₅GlcNAc₂ [2.0]. In particular embodiments, the plurality of N-linked glycans comprises greater than 60 or greater than 70 mole percent of an N-linked glycan consisting of Gal₄GlcNAc₂Man₃GlcNAc₂ [5.9]. In particular embodiments, the plurality of N-linked glycans comprises greater than 60 or greater than 70 mole percent of an N-linked glycan consisting of NANA₂Gal₂GlcNAc₂Man₃GlcNAc₂ [6.0].

The present invention further provides immunogenic compositions comprising a) a recombinant glycoprotein of the invention, wherein said glycoprotein is gD protein or immunogenic fragment thereof, b) a recombinant HSV gC protein or immunogenic fragment thereof, and c) an adjuvant; immunogenic compositions comprising a) a recombinant HSV gD protein or immunogenic fragment thereof, b) a recombinant glycoprotein of the invention, wherein said glycoprotein is gC protein or immunogenic fragment thereof, and c) an adjuvant; and immunogenic compositions comprising a) a recombinant glycoprotein of the invention, wherein said glycoprotein is gD protein or immunogenic fragment thereof, b) a recombinant glycoprotein of the invention, wherein said glycoprotein is gC protein or immunogenic fragment thereof, and c) an adjuvant.

In certain embodiments, the immunogenic compositions comprise gD protein and gC protein which are HSV-2 proteins. The immunogenic compositions may further comprise one or more recombinant HSV-2 proteins selected from a gB protein and a gE protein.

In certain embodiments, the adjuvant is a CpG-containing nucleotide molecule, an aluminum salt adjuvant, an ISCOM, or any combination thereof. In a particular embodiment, the adjuvant is a combination of an aluminum salt adjuvant and an ISCOM.

The present invention further provides methods for the production of recombinant Herpes Simplex Virus glycoprotein gC, gD, gB or gE comprising: a) transfecting an expression vector containing a DNA encoding a HSV protein selected from the group consisting of gC, gD, gB and gE, which is under regulation of a promoter functional in yeast, and which is directed for secretion by a signal sequence functional in yeast into a yeast host strain which has been engineered for heterologous protein expression through a combination of either knock-in or knock-out of protein folding chaperones, cargo receptor proteins or proteases; b) culturing said yeast in a medium; and c) recovering/isolating the HSV protein from the cultured yeast. In certain embodiments, the yeast is a methylotrophic yeast. In preferred embodiments the yeast is Pichia pastoris. In certain embodiments, the Pichia pastoris is a strain in which one or more of the following modifications have been made: i) disruption of the yeast protein disulfide isomerase gene (PDI); or ii) expression of one or more chaperones selected from the group consisting of human PDI, human calreticulin (hCRT), human Erp57, human FLC1, human ERO1, human FAD1. In particular embodiments, the yeast is a glycoengineered Pichia pastoris strain selected from 2.0, 5.9, and 6.0, which has one or more of the above modifications. In certain embodiments, the HSV glycoprotein is selected from gC, gB and gE.

The present invention further provides isolated DNA sequences encoding HSV glycoproteins which are codon-optimized for high level expression in yeast. In certain embodiments, the isolated nucleic acids comprise a nucleotide sequence of SEQ ID NO: 10, 12, 14, or 16. In one embodiment, the isolated nucleic acids further comprise an signal sequence and/or a HIS tag.

### BRIEF DESCRIPTION OF THE DRAWINGS

FIG. 1: Biacore and Western blot analysis of truncated forms of HSV-2 gC. Binding of Pichia gCt-2 to immobilized human C3b demonstrated by Biacore analysis (main figure). Western blot analysis of gCt-2 proteins (insert). Molecular mass standards (in kilodaltons) are indicated.

FIGS. 2A-B: Total and neutralizing antibody titers to HSV-2. (A) Pichia gCt-2 serum IgG titers to HSV-2 determined by ELISA. (B) Pichia gCt-2 neutralization titers to HSV-2 using heat-inactivated serum supplemented with 6.7% rabbit complement. Individual points represent the reciprocal dilution of the titer. The horizontal line indicates the geometric mean titer. Error bars indicate 95% confidence intervals.

FIGS. 3A-B: HSV-2 disease scores in guinea pigs vaccinated with gCt-2. Animals were scored for infection site (A) and urinary system disease (B) for 21 days p.i. (post infection). Disease scores are shown as the mean+standard error of the mean.

FIGS. 4A-B: Frequency and total number of herpetic lesions in guinea pigs vaccinated with gCt-2. (A) Lesion days, which are the days when clinically apparent herpetic lesions were observed on the perineum, were plotted cumulatively per day post-infection for Pichia gCt-2. (B) Total number of lesions per vaccine group. Lesions on the perineum of HSV-2-infected guinea pigs were quantified for 21 days and summed per vaccine group.

FIG. 5: Levels of HSV-2 shed in the vaginal tract of guinea pigs vaccinated with Pichia gCt-2. The results are shown as the mean+standard error of the mean.

FIG. 6: HSV-2 genome loads in lumbosacral DRG from guinea pigs vaccinated with Pichia gCt-2. Individual points represent the number of viral genomes per 1×10⁶ GAPDH gene copies. The horizontal line and error bars indicate the mean±standard error of the mean. LoQ (dotted line) and LoD (dashed line) indicate level of quantitation and level of detection of the PCR, respectively.

FIG. 7: Levels of C3b blocking activity of IgG from guinea pigs vaccinated with gCt-2. The results are shown as the mean OD₄₀₅+standard deviation.

FIG. 8: Inhibition of HSV-2 infection of Vero cell monolayers by soluble forms of Pichia-expressed gDt-2. The percent inhibition (100-[% β-gal⁺ cells in treated wells relative to the negative control]) was plotted as a function of gD-2 concentration.

FIGS. 9A-B: Endpoint antibody titers of mice vaccinated with 1 μg (A) or 3 μg (B) of gDt-2 proteins. Individual points represent the reciprocal dilution of the titer. The horizontal line indicates the geometric mean titer. Error bars indicate 95% confidence intervals.

FIGS. 10A-B: Survival curves of vaginally challenged mice that were vaccinated with 1 μg (A) or 3 μg (B) of various gDt-2 proteins.

FIGS. 11A-B: Total and neutralizing antibody titers to HSV-2. (A) Total IgG ELISA Geometric mean titers (GMT) against HSV-2 lysate. (B) 50% Neutralization GMTs to HSV-2 using heat-inactivated serum supplemented with 6.7% rabbit complement. Individual points represent the reciprocal dilution of the titer of individual animals. The horizontal line indicates the geometric mean titer. Error bars indicate 95% confidence intervals.

FIGS. 12A-D: HSV-2 disease scores in guinea pigs vaccinated with gD+gC. Animals were scored for total disease (A), vaginal disease (B), urinary system disease (C) and CNS disease (D) for 21 days p.i.

FIGS. 13A-B: Levels of HSV-2 shed in the vaginal tract of guinea pigs vaccinated with Pichia gD+gC.

FIG. 14: HSV-2 genome loads in lumbosacral DRG from vaccinated guinea pigs. Individual points represent the number of viral genomes per 1×10⁶ GAPDH gene copies. The horizontal line and error bars indicate the mean of each vaccine group±standard error of the mean. LoQ (dotted line) and LoD indicate the level of quantitation and level of detection of the PCR assay, respectively.

FIG. 15: Western blot analysis demonstrates that expression of HSV-2 gC is enhanced in P. pastoris host strains that have been engineered to express human chaperones. Lanes 1-4 are HSV-2 gC expressed in strain YGLY3812, a GFI5.9 host strain. Lane 5 is gC expressed in strain YGLY3669, a GFI2.0 P. pastoris host strain with the Pppdi gene deleted and expressing human PDI (hPDI), human Calreticulin, human ERP57, and human ERO1. Lane 6 is gC expressed in strain YGLY733, a GFI2.0 P. pastoris host strain. Lane 7 is gC expressed in strain YGLY3625, a GFI2.0 P. pastoris host strain with the Pppdi gene deleted and expressing hPDI. Lane 8 is gC expressed in strain YGLY3626, a GFI2.0 P. pastoris host strain with the Pppdi gene deleted and expressing hPDI.

### DETAILED DESCRIPTION OF THE INVENTION

The present invention provides recombinant Herpes Simplex Virus glycoproteins selected from the group consisting of gC, gD, gB and gE, or an immunogenic fragment thereof, comprising a plurality of bi-antennary N-linked glycans attached to the glycoprotein, or immunogenic fragment thereof, said plurality of N-linked glycans comprising greater than 50 mole percent of an N-linked glycan consisting of a structure selected from the group consisting of Man₅GlcNAc₂ [2.0]; NANA₂Gal₂GlcNAc₂Man₃GlcNAc₂ [6.0]; Gal₄GlcNAc₂Man₃GlcNAc₂ [5.9]; Gal₂GlcNAc₂Man₃GlcNAc₂; GlcNAc₂Man₃GlcNAc₂; Man₃GlcNAc₂; NANAGalGlcNAcMan₅GlcNAc₂; Gal₂GlcNAcMan₅GlcNAc₂; GalGlcNAcMan₅GlcNAc₂; and GlcNAcMan₅GlcNAc₂. In certain embodiments, a particular N-linked glycan is the predominant form. The recombinant glycoproteins of the invention are preferably produced in yeast such as Pichia pastoris which have been glycoengineered to provide the particular glycoform.

Expression in Pichia pastoris ensures reliable product quality, characterization and production. Glycoproteins have been expressed in CHO cells and baculovirus both which are difficult to express at large scale and result in an array and somewhat arbitrary decoration of sugars (glycans). The decoration pattern may not be consistently presented from lot to lot. Recently, significant advances have been made in engineering the yeast Pichia pastoris to introduce defined human glycosylation patterns into expressed proteins. Engineered Pichia pastoris have mammalian modified glycosylation with one specific glycoform per strain. Thus, the resulting expressed heterologous glycoprotein is uniformly decorated with sugars thereby standardizing the process and quality of vaccine antigens, e.g., expression of HSV-2 gD and gC with reproducible quality. In addition, specific glycosylated residues can be incorporated to potentially increase the immunogenicity of a given antigen. A protein-based vaccine with N-glycans containing terminal α-1,3-galactose has been shown previously to improve immunogenicity of such a molecule because humans have a high level of circulating antibodies directed against α-1,3-galactose residues. See International Patent Application No. PCT/US09/045,446, filed May 28, 2009; and Galili et al., 1984, J Exp Med 160:1519-31. Pichia is also a host system which is readily scale-able; proteins can be produced free of animals products and at low cost.

The present invention further provides immunogenic compositions comprising a) a recombinant glycoprotein of the invention, wherein said glycoprotein is gD protein or immunogenic fragment thereof, b) a recombinant HSV gC protein or immunogenic fragment thereof, and c) an adjuvant; immunogenic compositions comprising a) a recombinant HSV gD protein or immunogenic fragment thereof, b) a recombinant glycoprotein of the invention, wherein said glycoprotein is gC protein or immunogenic fragment thereof, and c) an adjuvant; and immunogenic compositions comprising a) a recombinant glycoprotein of the invention, wherein said glycoprotein is gD protein or immunogenic fragment thereof, b) a recombinant glycoprotein of the invention, wherein said glycoprotein is gC protein or immunogenic fragment thereof, and c) an adjuvant. Unless specified as a recombinant glycoprotein of the invention, a recombinant HSV glycoprotein may be from any source known in the art. In certain embodiments, the immunogenic compositions comprise gD protein and gC protein which are HSV-2 glycoprotein, HSV-1 glycoproteins, or any combination thereof. The immunogenic compositions may further comprise one or more recombinant HSV-2 glycoproteins or recombinant HSV-1 glycoproteins selected from a gB protein and a gE protein.

A combination vaccine of glycoprotein ectodomains including gD and gC would be useful for the prophylactic prevention of HSV infection and disease and reduction of viral load and shedding. Inclusion of Merck aluminum adjuvant plus Iscomatrix® is an example of a combination of adjuvants for including in a vaccine.

As used herein, “HSV protein” refers to an HSV-1 or HSV-2 protein. Thus, any reference to HSV in the composition and methods of the present invention may refer to HSV-1, HSV-2, both HSV-1 and HSV-2, or HSV-1 or HSV-2.

As used herein, “HSV-1” refers to a Herpes Simplex Virus-1. The term may refer to any HSV-1 strain known in the art including, but not limited to, KOS, F, NS, CL101, “17”, “17+syn”, MacIntyre, McKrae, MP, or HF.

As used herein, “HSV-2” refers to a Herpes Simplex Virus-2. The term may refer to any HSV-2 strain known in the art including, but not limited to, 333, 2.12, HG52, MS, G, or 186.

As used herein, the terms “N-glycan” and “glycoform” are used interchangeably and refer to an N-linked oligosaccharide, e.g., one that is attached by an asparagine-N-acetylglucosamine linkage to an asparagine residue of a polypeptide. N-linked glycoproteins contain an N-acetylglucosamine residue linked to the amide nitrogen of an asparagine residue in the protein. The predominant sugars found on glycoproteins are glucose, galactose, mannose, fucose, N-acetylgalactosamine (GalNAc), N-acetylglucosamine (GlcNAc) and sialic acid (e.g., N-acetyl-neuraminic acid (NANA)). The processing of the sugar groups occurs co-translationally in the lumen of the ER and continues post-translationally in the Golgi apparatus for N-linked glycoproteins.

N-glycans have a common pentasaccharide core of Man₃GlcNAc₂ (“Man” refers to mannose; “Glc” refers to glucose; and “NAc” refers to N-acetyl; GlcNAc refers to N-acetylglucosamine). N-glycans differ with respect to the number of branches (antennae) comprising peripheral sugars (e.g., GlcNAc, galactose, fucose and sialic acid) that are added to the Man₃GlcNAc₂ (“Man₃”) core structure which is also referred to as the “triammnose core”, the “pentasaccharide core” or the “paucimannose core”. N-glycans are classified according to their branched constituents (e.g., high mannose, complex or hybrid). A “high mannose” type N-glycan has five or more mannose residues. A “complex” type N-glycan typically has at least one GlcNAc attached to the 1,3 mannose arm and at least one GlcNAc attached to the 1,6 mannose arm of a “trimannose” core. Complex N-glycans may also have galactose (“Gal”) or N-acetylgalactosamine (“GalNAc”) residues that are optionally modified with sialic acid or derivatives (e.g., “NANA” or “NeuAc”, where “Neu” refers to neuraminic acid and “Ac” refers to acetyl). Complex N-glycans may also have intrachain substitutions comprising “bisecting” GlcNAc and core fucose (“Fuc”). Complex N-glycans may also have multiple antennae on the “trimannose core,” often referred to as “multiple antennary glycans.” A “hybrid” N-glycan has at least one GlcNAc on the terminal of the 1,3 mannose arm of the trimannose core and zero or more mannoses on the 1,6 mannose arm of the trimannose core. The various N-glycans are also referred to as “glycoforms.”

Abbreviations used herein are of common usage in the art, see, e.g., abbreviations of sugars, above. Other common abbreviations include “PNGase”, or “glycanase” or “glucosidase” which all refer to peptide N-glycosidase F (EC 3.2.2.18).

When referring to “mole percent” of a glycan present in a preparation of a glycoprotein, the term means the molar percent of a particular glycan present in the pool of N-linked oligosaccharides released when the protein preparation is treated with PNG'ase and then quantified by a method that is not affected by glycoform composition, (for instance, labeling a PNG'ase released glycan pool with a fluorescent tag such as 2-aminobenzamide and then separating by high performance liquid chromatography or capillary electrophoresis and then quantifying glycans by fluorescence intensity). For example, 50 mole percent NANA₂Gal₂GlcNAc₂Man₃GlcNAc₂ means that 50 percent of the released glycans are NANA₂Gal₂GlcNAc₂Man₃GlcNAc₂ and the remaining 50 percent are comprised of other N-linked oligosaccharides. In various embodiments, the mole percent of a particular glycan in a preparation of glycoprotein will be between 20% and 100%, preferably above 25%, 30%, 35%, 40% or 45%, more preferably above 50%, 55%, 60%, 65% or 70% and most preferably above 75%, 80% 85%, 90% or 95%.

As used herein, the term “predominantly” or variations such as “the predominant” or “which is predominant” will be understood to mean the glycan species that has the highest mole percent (%) of total N-glycans after the glycoprotein has been treated with PNGase and released glycans analyzed by mass spectroscopy, for example, MALDI-TOF MS. In other words, the phrase “predominantly” is defined as an individual entity, such as a specific glycoform, is present in greater mole percent than any other individual entity. For example, if a composition consists of species A in 40 mole percent, species B in 35 mole percent and species C in 25 mole percent, the composition comprises predominantly species A

As used herein, “administering” or “administration” refers to directly introducing into a subject by injection or other means a composition of the present invention. “Administering” or “administration” may also refer to contacting a cell of the subject's immune system with a vaccine or recombinant HSV protein or mixture thereof.

As used herein, “DRG” refers to a neuronal cell body and, in particular, contains the neuron cell bodies of nerve fibers. The term may also refer to any other definition of “DRG” used in the art, for example, dorsal root ganglia, also a site for HSV latency.

As used herein, “TG” refers to trigeminal ganglia, which is the normal site latency for HSV-1.

As used herein, the term “fragment” is used herein to refer to a protein or polypeptide that is shorter or comprises fewer amino acids than the full length protein or polypeptide. In another embodiment, fragment refers to a nucleic acid that is shorter or comprises fewer nucleotides than the full length nucleic acid. A fragment may be an N-terminal fragment, a C-terminal fragment, a fragment completely within the full-length sequence, i.e., having one or more residues deleted at both ends, or an internally deleted fragment, i.e., having one or more residues deleted within the full-length sequence, or any combination thereof. A fragment of an HSV glycoprotein has one or more glycosylation sites for presenting a particular N-linked glycan.

As used herein, a “flare” or “recurrence” refers to reinfection of skin tissue following latent neuronal HSV infection. The terms may also refer to reactivation of HSV after a latency period and/or symptomatic HSV lesions following a non-symptomatic latency period.

As used herein, “HSV encephalitis” refers to an encephalitis caused by a Herpes Simplex Virus-1 (HSV-1). The term may also refer to an encephalitis associated with HSV or any other type of HSV-mediated encephalitis known in the art.

As used herein, an “immunogenic fragment” refers to a portion of a polypeptide or protein that is immunogenic and elicits a protective immune response when administered to a subject.

As used herein, “immunogenicity” or “immunogenic” refers to the innate ability of a protein, peptide, nucleic acid, antigen or organism to elicit an immune response in an animal when the protein, peptide, nucleic acid, antigen or organism is administered to the animal.

As used herein, the terms “impeding a HSV infection” and “impeding a primary HSV infection” refer to decreasing the titer of infectious virus by a medically significant amount, for example, 50%, 55%, 60%, 65%, 70%, 75%, 80%, 85%, 90%, 92%, 95%, 96%, 97%, 98%, 99% or over 99%. The terms may also refer to decreasing the extent of viral replication by a medically significant amount, for example, 50%, 55%, 60%, 65%, 70%, 75%, 80%, 85%, 90%, 92%, 95%, 96%, 97%, 98%, 99% or over 99%.

As used herein, “preventing” refers, inter alia, to delaying the onset of symptoms, preventing relapse to a disease, decreasing the number or frequency of relapse episodes, increasing latency between symptomatic episodes, or a combination thereof. “Suppressing” or “inhibiting”, refers, inter alia, to reducing the severity of symptoms, reducing the severity of an acute episode, reducing the number of symptoms, reducing the incidence of disease-related symptoms, reducing the latency, ameliorating symptoms, reducing secondary symptoms, reducing secondary infections, prolonging patient survival, or a combination thereof.

As used herein, “treating” refers to either therapeutic treatment or prophylactic or preventative measures, wherein the object is to prevent or lessen the targeted pathologic condition or disorder as described hereinabove. Thus, in one embodiment, treating may include directly affecting or curing, suppressing, inhibiting, preventing, reducing the severity of, delaying the onset of, reducing symptoms associated with the disease, disorder or condition, or a combination thereof. Thus, in one embodiment, “treating” refers, inter alia, to delaying progression, expediting remission, inducing remission, augmenting remission, speeding recovery, increasing efficacy of or decreasing resistance to alternative therapeutics, or a combination thereof.

As used herein, “zosteriform” refers to skin lesions characteristic of an HSV infection, particularly during reactivation infection, which begin as a rash and follow a distribution near dermatomes, commonly occurring in a strip or belt-like pattern. In one embodiment, the rash evolves into vesicles or small blisters filled with serous fluid. In one embodiment, zosteriform lesions form in mice as a result of contact with HSV. In another embodiment, zosteriform lesions form in humans as a result of contact with HSV.

As used herein, “zosteriform spread” refers to an HSV infection that spreads from the ganglia to secondary skin sites within the dermatome. The term may also refer to spread within the same dermatome as the initial site of infection or any other definition of “zosteriform spread” known in the art.

### HSV Glycoproteins

The present invention provides compositions of a recombinant Herpes Simplex Virus glycoprotein selected from the group consisting of gC, gD, gB and gE, or an immunogenic fragment thereof, wherein the glycoprotein is mostly (greater than 50%) and/or predominantly a specific N-linked glycan. The N-linked glycan includes, but is not limited to, Man₅GlcNAc₂ [2.0]; Gal₄GlcNAc₂Man₃GlcNAc₂ [5.9]; NANA₂Gal₂GlcNAc₂Man₃GlcNAc₂ [6.0]; Gal₂GlcNAc₂Man₃GlcNAc₂; GlcNAc₂Man₃GlcNAc₂; GlcNAcMan₅GlcNAc₂; GlcNAcMan₅GlcNAc₂; Man₃GlcNAc₂; NANAGalGlcNAcMan₅GlcNAc₂; GalGlcNAcMan₅GlcNAc₂; Gal₂GlcNAcMan₅GlcNAc₂; GlcNAcMan₅GlcNAc₂; and Man₈GlcNAc₂.

Particularly preferred ectodomains, or fragments thereof, include, but are not limited to, gC-1, gC-2, gD-1, gD-2, gB-1, gB-2, gE-I, and gE-2, as well as immunogenic fragments of these gC, gD, gB and gE glycoproteins. Such immunogenic fragments may comprise, or alternatively, consist of, an ectodomain, or a fragment thereof. Representative HSV glycoprotein fragments are provided in U.S. Patent Application Publication No. 2009/0098162, incorporated by reference herein in its entirety.

In certain embodiments, the present invention relates to the production of an HSV glycoprotein ectodomain in yeast, particularly Pichia pastoris.

The invention provides methods and materials for the transformation, expression and selection of recombinant HSV glycoproteins, particularly gC, gD, gB and gE, and immunogenic fragments thereof, in lower eukaryotic host cells, which have been genetically engineered to produce glycoproteins with specific desired N-glycans as the predominant species.

In certain embodiments, the plurality of N-linked glycans comprises greater than 50 mole percent of an N-linked glycan consisting of a structure selected from the group consisting of Man₅GlcNAc₂ [2.0] and NANA₂Gal₂GlcNAc₂Man₃GlcNAc₂ [6.0]. In particular embodiments, the plurality of N-linked glycans comprises greater than 75 or greater than 85 mole percent of an N-linked glycan consisting essentially of Man₅GlcNAc₂ [2.0]. In particular embodiments, the plurality of N-linked glycans comprises greater than 60 or greater than 70 mole percent of an N-linked glycan consisting essentially of NANA₂Gal₂GlcNAc₂Man₃GlcNAc₂ [6.0].

In one embodiment, the gC-1 protein utilized in methods and compositions of the present invention has the sequence:

(SEQ ID NO: 1)
MAPGRVGLAVVLWGLLWLGAGVAGGSETASTGPTITAGAVTNASEAPTSGSPGSAASP
EVTPTSTPNPNNVTQNKTTPTEPASPPTTPKPTSTPKSPPTSTPDPKPKNNTTPAKSGRPTK
PPGPVWCDRRDPLARYGSRVQIRCRFRNSTRMEFRLQIWRYSMGPSPPIAPAPDLEEVLT
NITAPPGGLLVYDSAPNLTDPHVLWAEGAGPGADPPLYSVTGPLPTQRLIIGEVTPATQG
MYYLAWGRMDSPHEYGTWVRVRMFRPPSLTLQPHAVMEGQPFKATCTAAAYYPRNP
VEFDWFEDDRQVFNPGQIDTQTHEHPDGFTTVSTVTSEAVGGQVPPRTFTCQMTWHRD
SVTFSRRNATGLALVLPRPTTTMEFGVRHVVCTAGCVPEGVTFAWFLGDDPSPAAKSAV
TAQESCDHPGLATVRSTLPISYDYSEYICRLTGYPAGIPVLEHHGSHQPPPRDPTERQVIE
AIEWVGIGIGVLAAGVLVVTAIVYVVRTSQSRQRHRR.

In another embodiment, a gC-1 protein utilized in methods and compositions of the present invention is a homologue, isoform or variant of SEQ ID NO: 1. For example, a gC-1 protein may have 95% or greater homology to SEQ ID NO: 1 or may be a variant having up to 20 amino acid changes (any combination of additions, deletions or substitutions). In another embodiment, the protein is an immunogenic fragment of any of SEQ ID NO: 1, a homologue of SEQ ID NO: 1, an isoform of SEQ ID NO: 1, or a variant of SEQ ID NO: 1.

In another embodiment, the nucleic acid sequence encoding a gC-1 protein utilized in methods and compositions of the present invention is set forth in a GenBank entry having one of the following Accession Numbers: NCJ301806, X14112, AJ421509, AJ421508, AJ421507, AJ421506, AJ421505, AJ421504, AJ421503, AJ421502, AJ421501, AJ421500, AJ421499, AJ421498, AJ421497, AJ421496, AJ421495, AJ421494, AJ421493, AJ421492, AJ421491, AJ421490, AJ421489, AJ421488, and AJ421487. In another embodiment, the gC-1 protein is encoded by a nucleotide molecule having a sequence set forth in one of the above GenBank entries. In another embodiment, the protein is a homologue, isoform, variant or fragment of a protein encoded by a sequence set forth in one of the above GenBank entries. In another embodiment, the protein is a fragment of any of a homologue, isoform, or variant of a protein encoded by a sequence set forth in one of the above GenBank entries.

In one embodiment, a gC-2 protein utilized in methods and compositions of the present invention has the sequence:

(SEQ ID NO: 2)
MALGRVGLAVGLWGLLWVGVVVVLANASPGRTTTVGPRGNASNAAPSASPRNASAPR
TTPTPPQPRKATKSKASTAKPAPPPKTGPPKTSSEPVRCNRHDPLARYGSRVQIRCRFPNS
TRTEFRLQIWRYATATDAEIGTAPSLEEVMVNVSAPPGGQLVYDSAPNRTDPHVIWAEG
AGPGASPRLYSVVGPLGRQRLIIEELTLETQGMYYWVWGRTDRPSAYGTWVRVRVFRP
PSLTIHPHAVLEGQPFKATCTAATYYPGNRAEFVWFEDGRRVFDPAQIHTQTQENPDGFS
TVSTVTSAAVGGQGPPRTFTCQLTWHRDSVSFSRRNASGTASVLPRPTITMEFTGDHA
VCTAGCVPEGVTFAWFLGDDSSPAEKVAVASQTSCGRPGTATIRSTLPVSYEQTEYICRL
AGYPDGIPVLEHHGSHQPPPRDPTERQVIRAVEGAGIGVAVLVAVVLAGTAVVYLTHAS
SVRYRRLR.

In another embodiment, a gC-2 protein utilized in methods and compositions of the present invention is a homologue, isoform or variant of SEQ ID NO: 2. For example, a gC-2 protein may have 95% or greater homology to SEQ ID NO: 2 or may be a variant having up to 20 amino acid changes (any combination of additions, deletions or substitutions). In another embodiment, the protein is an immunogenic fragment of any of SEQ ID NO: 2, a homologue of SEQ ID NO: 2, an isoform of SEQ ID NO: 2, or a variant of SEQ ID NO: 2.

In another embodiment, the nucleic acid sequence encoding a gC-2 protein utilized in methods and compositions of the present invention is set forth in a GenBank entry having one of the following Accession Numbers: NCJ301798, Z86099, M10053, AJ297389, AF021341, U12179, U12177, U12176, and U12178. In another embodiment, the gC-2 protein is encoded by a nucleotide molecule having a sequence set forth in one of the above GenBank entries. In another embodiment, the protein is a homologue, isoform, or variant of a protein encoded by a sequence set forth in one of the above GenBank entries. In another embodiment, the protein is a fragment of any of a homologue, isoform, or variant of a protein encoded by a sequence set forth in one of the above GenBank entries.

In one embodiment, the gC protein fragment utilized in methods and compositions of the present invention is an immunogenic fragment. In another embodiment, mutants, sequence conservative variants, and functional conservative variants of gC are useful in methods and compositions of the present invention, provided that all such variants retain the required immunoprotective effect.

In another embodiment, the immunogenic fragment can comprise an immunoprotective gC antigen from any strain of HSV. In another embodiment, the immunogenic fragment can comprise sequence variants of HSV, as found in infected individuals.

In another embodiment, the gC protein fragment comprises, or consists of, a gC immune evasion domain, or a portion thereof. In another embodiment, the gC protein fragment is a C3b-binding domain or portion thereof. In another embodiment, “C3b-binding domain” refers to a domain that mediates binding of gC with a host C3b molecule or a domain that mediates interaction of gC with a host C3b molecule.

In another embodiment, the gC protein fragment is a properdin interfering domain, or a portion thereof “Properdin-interfering domain” refers to a domain that blocks or inhibits binding or interaction of a host C3b molecule with a host properdin molecule. In another embodiment, the gC domain is any other gC domain known in the art to interfere with binding of a host C3b molecule with a host properdin molecule.

In another embodiment, the gC protein fragment is a C5 interfering domain, or a portion thereof. “C5-interfering domain” refers to a domain that interferes with binding or interaction of a host C3b molecule with a host C5 molecule. In another embodiment, the gC domain is any other gC domain known in the art to interfere with or inhibit binding or interaction of a host C3b molecule with a host C5 molecule.

In another embodiment, (e.g. in the case of gC-1), the gC domain consists of approximately AA 27-475, approximately AA 27-383, approximately AA 78-475, approximately AA78-383, or approximately AA 124-383.

In another embodiment, (e.g. in the case of gC-2), the gC domain consists of approximately AA 24-444, approximately AA 24-353, approximately AA 51-444, approximately AA 51-353, or approximately AA 93-354.

In another embodiment, the gC-1 or gC-2 protein, or fragment thereof, is modified with an antigenic tag. In another embodiment, the tag is a histidine (“His”) tag. In another embodiment, the His tag consists of 5, 6 or 9 histidine residues. In another embodiment, the His tag consists of another number of histidine residues. In one embodiment, the gC-1 fragment is AA 26-457 modified with a His tag. In another embodiment, the gC-2 fragment is AA 27-426 modified with a His tag.

In one embodiment, the gD-1 protein utilized in methods and compositions of the present invention has the sequence:

(SEQ ID NO: 3)
MGGAAARLGAVILFVVIVGLHGVRGKYALADASLKLADPNRFRRKDLPVLDQLTDPPG
VRRVYHIQAGLPDPFQPPSLPITVYYAVLERACRSVLLNAPSEAPQIVRGASEDVRKQPY
NLTIAWFRMGGNCATPITVMEYTECSYNKSLGACPIRTQPRWNYYDSFSAVSEDNLGFL
MHAPAFETAGTYLRLVKINDWTEITQFILEHRAKGSCKYALPLRIPPSACLSPQAYQQGV
TVDSIGMLPRFIPENQRTVAVYSLKIAGWHGPKAPYTSTLLPPELSETPNATQPELAPEAP
EDSALLEDPVGTVAPQIPPNWHIPSIQDAATPYHPPATPNNMGLIAGAVGGSLLAALVICG
IV YWMRRRTQKAPKRIRLPHIREDDQPSSHQPLFY.

In another embodiment, a gD-1 protein utilized in methods and compositions of the present invention is a homologue, isoform or variant of SEQ ID NO: 3. For example, a gD-1 protein may have 95% or greater homology to SEQ ID NO: 3 or may be a variant having up to 20 amino acid changes (any combination of additions, deletions or substitutions). In another embodiment, the gD-1 protein is an immunogenic fragment of any of SEQ ID NO: 3, a homologue of SEQ ID NO: 3, an isoform of SEQ ID NO: 3, or a variant of SEQ ID NO: 3.

In another embodiment, the nucleic acid sequence encoding a gD-1 protein utilized in methods and compositions of the present invention is set forth in a GenBank entry having one of the following Accession Numbers: NC_{—}001806, X14112, E03111, E03023, E02509, E00402, E00401, E00395, AF487902, AF487901, AF293614, L09242, J02217, L09244, L09245, and L09243. In another embodiment, the gD-1 protein is encoded by a nucleotide molecule having a sequence set forth in one of the above GenBank entries. In another embodiment, the gD-1 protein is a homologue, isoform or variant of a protein encoded by a sequence set forth in one of the above GenBank entries. In another embodiment, the gD-1 protein is a fragment of any of a protein encoded by a sequence set forth in one of the above GenBank entries, a homologue of a protein encoded by a sequence set forth in one of the above GenBank entries, an isoform of a protein encoded by a sequence set forth in one of the above GenBank entries, or a variant of a protein encoded by a sequence set forth in one of the above GenBank entries.

In another embodiment, the gD-1 fragment consists of about amino acids (AA) 26-306, about AA 26-310, about AA 26-331, about AA 26-339, or about AA 26-339 and, optionally, containing one or more of mutations V62C and A327C.

In another embodiment, the gD protein fragment of methods and compositions of the present invention is a gD-2 fragment. In another embodiment, the gD-2 fragment consists of, or alternatively comprises, the gD-2 ectodomain, or a fragment thereof. In another embodiment, the gD-2 fragment is any other gD-2 fragment known in the art.

In one embodiment, the gD-2 protein utilized in methods and compositions of the present invention has the sequence:

(SEQ ID NO: 4)
MGRLTSGVGTAALLVVAVGLRVVCAKYALADPSLKMADPNRFRGKNLPVLDQLTDPP
GVKRVYHIQPSLEDPFQPPSIPITVYYAVLERACRSVLLHAPSEAPQIVRGASDEARKHTY
NLTIAWYRMGDNCAIPITVMEYTECPYNKSLGVCPIRTQPRWSYYDSFSAVSEDNLGFL
MHAPAFETAGTYLRLVKINDWTETTQFILEHRARASCKYALPLRIPPAACLTSKAYQQGV
TVDSIGMLPRFIPENQRTVALYSLKIAGWHGPKPPYTSTLLPPELSDTTNATQPELVPEDP
EDSALLEDPAGTVSSQIPPNWHIPSIQDVAPHHAPAAPSNPGLIIGALAGSTLAVLVIGGIA
FWVRRRAQMAPKRLRLPHIRDDDAPPSHQPLFY.

In another embodiment, a gD-2 protein utilized in methods and compositions of the present invention is a homologue, isoform or variant of SEQ ID NO: 4. For example, a gD-2 protein may have 95% or greater homology to SEQ ID NO: 4 or may be a variant having up to 20 amino acid changes (any combination of additions, deletions or substitutions). In another embodiment, the protein is an immunogenic fragment of any of SEQ ID NO: 4, a homologue of SEQ ID NO: 4, an isoform of SEQ ID NO: 4, or a variant of SEQ ID NO: 4.

In another embodiment, the nucleic acid sequence encoding a gD-2 protein utilized in methods and compositions of the present invention is set forth in a GenBank entry having one of the following Accession Numbers: NC_{—}001798, E00205, Z86099, AY779754, AY779753, AY779752, AY779751, AY779750, AY517492, AY155225, and K01408. In another embodiment, the gD-2 protein is encoded by a nucleotide molecule having a sequence set forth in one of the above GenBank entries. In another embodiment, the protein is a homologue, isoform, or variant of a protein encoded by a sequence set forth in one of the above GenBank entries. In another embodiment, the protein is a fragment of any of a protein encoded by a sequence set forth in one of the above GenBank entries, a homologue of a protein encoded by a sequence set forth in one of the above GenBank entries an isoform of a protein encoded by a sequence set forth in one of the above GenBank entries, or a variant of a protein encoded by a sequence set forth in one of the above GenBank entries.

In another embodiment, the gD-2 fragment consists of about AA 26-306, about AA 26-310, about AA 26-331, about AA 26-339, or about AA 26-339 and, optionally, containing one or more of the mutations V62C and V327C.

In another embodiment, the recombinant gD protein, or fragment thereof, elicits antibodies that inhibit binding of gD to a cellular receptor. A cellular receptor is selected from herpesvirus entry mediator A (HveA/HVEM), nectin-1 (HveC), nectin-2 (HveB), a modified form of heparan sulfate, a heparan sulfate proteoglycan or any other gD receptor known in the art.

In another embodiment, the recombinant gD protein or fragment thereof includes AA 26-57. In another embodiment, inclusion of these residues elicits antibodies that inhibit binding to HVEM. In another embodiment, the gD protein or fragment includes P246. In another embodiment, the recombinant gD protein or fragment includes a residue selected from Y63, R159, D240, and P246. In another embodiment, inclusion of one of these residues elicits antibodies that inhibit binding to nectin-1.

The above nomenclature for gD AA residues includes the residues of the signal sequence. Thus, in one embodiment, residue one of the mature protein is referred to as “26”.

In another embodiment, a gD protein fragment utilized in methods and compositions of the present invention is an immunogenic fragment. In another embodiment, mutants, sequence conservative variants, and functional conservative variants of gD are useful in methods and compositions of the present invention, provided that all such variants retain the required immuno-protective effect.

In another embodiment, the immunogenic fragment can comprise an immuno-protective gD antigen from any strain of HSV. In another embodiment, the immunogenic fragment can comprise sequence variants of HSV, as found in infected individuals.

In one embodiment, the gE-1 protein utilized in methods and compositions of the present invention has the sequence:

(SEQ ID NO: 5)
MDRGAVVGFLLGVCVVSCLAGTPKTSWRRVSVGEDVSLLPAPGPTGRGPTQKLLWAVE
PLDGCGPLHPSWVSLMPPKQVPETVVDAACMRAPVPLAMAYAPPAPSATGGLRTDFV
WQERAAVVNRSLVIYGVRETDSGLYTLSVGDIKDPARQVASVVLVVQPAPVPTPPPTPA
DYDEDDNDEGEGEDESLAGTPASGTPRLPPSPAPPRSWPSAPEVSHVRGVTVRMETPEAI
LFSPGEAFSTNVSIHAIAHDDQTYTMDVVWLRFDVPTSCAEMRIYESCLYHPQLPECLSP
ADAPCAASTWTSRLAVRSYAGCSRTNPPPRCSAEAHMEPFPGLAWQAASVNLEFRDAS
PQHSGLYLCVVYVNDHIHAWGHTTINTAAQYRNAVVEQPLPQRGADLAEPTHPHVGAPP
HAPPTHGALRLGAVMGAALLLSALGLSVWACMTCWRRRAWRAVKSRASGKGPTYIRV
ADSELYADWSSDSEGERDQVPWLAPPERPDSPSTNGSGFEILSPTAPSVYPRSDGHQSRR
QLTTFGSGRPDRRYSQASDSSVFW.

In another embodiment, a gE-1 protein utilized in methods and compositions of the present invention is a homologue, isoform, or variant of SEQ ID NO: 5. For example, a gE-1 protein may have 95% or greater homology to SEQ ID NO: 5 or may be a variant having up to 20 amino acid changes (any combination of additions, deletions or substitutions). In another embodiment, the protein is an immunogenic fragment of any of SEQ ID NO: 5, a homologue of SEQ ID NO: 5, an isoform of SEQ ID NO: 5, or a variant of SEQ ID NO: 5.

In another embodiment, the nucleic acid sequence encoding a gE-1 protein utilized in methods and compositions of the present invention is set forth in a GenBank entry having one of the following Accession Numbers: NC_{—}001806, X14112, DQ889502, X02138, and any of AJ626469-AJ626498. In another embodiment, the gE-1 protein is encoded by a nucleotide molecule having a sequence set forth in one of the above GenBank entries. In another embodiment, the protein is a homologue of a protein encoded by a sequence set forth in one of the above GenBank entries, an isoform of a protein encoded by a sequence set forth in one of the above GenBank entries, or a variant of a protein encoded by a sequence set forth in one of the above GenBank entries. In another embodiment, the protein is a fragment of any of a protein encoded by a sequence set forth in one of the above GenBank entries, a homologue of a protein encoded by a sequence set forth in one of the above GenBank entries, an isoform of a protein encoded by a sequence set forth in one of the above GenBank entries, or a fragment of a variant of a protein encoded by a sequence set forth in one of the above GenBank entries.

In one embodiment, the gE-2 protein utilized in methods and compositions of the present invention has the sequence:

(SEQ ID NO: 6)
MARGAGLVFFVGVWVVSCLAAAPRTSWKRVTSGEDVVLLPAPAERTRAHK
LLWAAEPLDACGPLRPSWVALWPPRRVLETVVDAACMRAPEPLAIAYSPP
FPAGDEGLYSELAWRDRVAVVNESLVIYGALETDSGLYTLSVVGLSDEAR
QVASVVLVVEPAPVPTPTPDDYDEEDDAGVTNARRSAFPPQPPPRRPPVA
PPTHPRVIPEVSHVRGVTVHMETLEAILFAPGETFGTNVSIHAIAHDDGP
YAMDVVWMRFDVPSSCADMRIYEACLYHPQLPECLSPADAPCAVSSWA
YRLAVRS YAGCSRTTPPPRCFAEARMEPVPGLAWLASTVNLEFQHASPQ
HAGLYLCVVYVDDHIHAWGHMTISTAAQYRNAVVEQHLPQRQPEPVEPTR
PHVRAPHPAPSARGPLRLGAVLGAALLLAALGLSAWACMTCWRRRSWRA
VKSRASATGPTY1RVADSEL YADWSSDSEGERDGSLWQDPPERPDSPST
NGSGFEILSPTAPSVYPHSEGRKSRRPLTTFGSGSPGRRHSQASYPSVL
W.

In another embodiment, a gE-2 protein utilized in methods and compositions of the present invention is a homologue, isoform or variant of SEQ ID NO: 6. For example, a gE-2 protein may have 95% or greater homology to SEQ ID NO: 6 or may be a variant having up to 20 amino acid changes (any combination of additions, deletions or substitutions). In another embodiment, the protein is an immunogenic fragment of any of SEQ ID NO: 6, a homologue of SEQ ID NO: 6, an isoform of SEQ ID NO: 6, or a variant of SEQ ID NO: 6.

In another embodiment, the nucleic acid sequence encoding a gE-2 protein utilized in methods and compositions of the present invention is set forth in a GenBank entry having one of the following Accession Numbers: NC_{—}001798, Z86099, D00026, X04798, and M 14886. In another embodiment, the gE-2 protein is encoded by a nucleotide molecule having a sequence set forth in one of the above GenBank entries. In another embodiment, the protein is a homologue, isoform or variant of a protein encoded by a sequence set forth in one of the above GenBank entries. In another embodiment, the protein is a fragment of any of a protein encoded by a sequence set forth in one of the above GenBank entries, a homologue of a protein encoded by a sequence set forth in one of the above GenBank entries, an isoform of a protein encoded by a sequence set forth in one of the above GenBank entries, or a variant of a protein encoded by a sequence set forth in one of the above GenBank entries.

In another embodiment, a gE fragment utilized in methods and compositions of the present invention comprises an IgG Fc-binding domain of the gE protein. In another embodiment, the gE fragment comprises AA 24-224, or a portion thereof. In another embodiment, the portion is sufficient to elicit antibodies that block immune evasion by the IgG Fc-binding domain of the gE protein.

In another embodiment, the gE fragment comprises a portion of a gE IgG Fc-binding domain. In another embodiment, (e.g. in the case of gE-1) the gE domain consists of about AA 24-409. In another embodiment, the domain consists of about 24-224. In alternative embodiments, the gE domain consists essentially of, or comprises, any of the specified amino acid residue ranges. In another embodiment, the gE domain is any other gE domain known in the art to mediate binding to IgG Fc.

In another embodiment, the gE protein comprises a gE domain involved in cell-to-cell spread. In another embodiment, the gE domain consists approximately of AA 256-291. In another embodiment, the gE domain consists approximately of AA 348-380. In another embodiment, the gE domain includes AA 380. In another embodiment, the gE domain is any other gE domain known in the art to be involved in cell-to-cell spread. In another embodiment, the gE domain is known to facilitate cell-to-cell spread. In another embodiment, the gE domain is known to be required for cell-to-cell spread.

In another embodiment, a gE fragment utilized in methods and compositions of the present invention is an immunogenic fragment. In another embodiment, the immunogenic fragment can comprise an immuno-protective gE antigen from any strain of HSV. In another embodiment, the immunogenic fragment can comprise sequence variants of HSV, as found in infected individuals.

In another embodiment, the gE fragment comprises an immune evasion domain, or a portion of an immune evasion domain. In another embodiment, an HSV-1 gE AA sequence is utilized. In another embodiment, an HSV-1 gE protein or peptide is utilized.

In another embodiment, (e.g. in the case of gE-2) the gE protein fragment consists of about AA 21-416. In another embodiment, the range is any of the ranges specified in the preceding paragraphs.

“Immune evasion domain” refers to a domain that interferes with or reduces in vivo anti-HSV efficacy of anti-HSV antibodies (e.g. anti-gD antibodies). In another embodiment, the domain interferes or reduces in vivo anti-HSV efficacy of an anti-HSV immune response. In another embodiment, the domain reduces the immunogenicity of an HSV protein (e.g. gD) during subsequent infection. In another embodiment, the domain reduces the immunogenicity of an HSV protein during subsequent challenge. In another embodiment, the domain reduces the immunogenicity of HSV during subsequent challenge. In another embodiment, the domain reduces the immunogenicity of an HSV protein in the context of ongoing HSV infection. In another embodiment, the domain reduces the immunogenicity of HSV in the context of ongoing HSV infection.

In another embodiment, the gE protein, or fragment thereof, is modified with an antigenic tag. In another embodiment, the tag is a histidine (“His”) tag, preferably consisting of 5, 6 or 9 histidine residues. In another embodiment, the gE fragment utilized in methods and compositions of the present invention is approximately AA 24-409 with a 6 His tag at the C-terminus.

In another embodiment, the gE-1 fragment consists of about AA 26-411, about AA 213-390. Fragments may contain a C359A mutation.

In another embodiment, the gE-2 fragment consists of about AA 26-406, about AA 208-385. Fragments may contain a C354A mutation.

In one embodiment, the gB-1 protein utilized in methods and compositions of the present invention has the sequence:

(SEQ ID NO: 7)
MHQGAPSWGRRWFVVWALLGLTLGVLVASAAPTSPGTPGVAAATQAANGGPATPAPP
PLGAAPTGDPKPKKNKKPKNPTPPRPAGDNATVAAGHATLREHLRDIKAENTDANFYV
CPPPTGATVVQFEQPRRCPTRPEGQNYTEGIAVVFKENIAVYKFKATMYYKDVTVSQVW
FGHRYSQFMGIFEDRAPVPFEEVIDKINAKGVCRSTAKYVRNNLETTAFFIRDDHETDME
LKPANAATRTSRGWHTTDLKYNPSRVEAFHRYGTTVNCIVEEVDARSVYPYDEFVLAT
GDFVYMSPFYGYREGSHTEHTTYAADRFKQVDGFYARDLTTKARATAPTTRNLLTTPK
FTVAWDWVPKRPSVCTMTKWQEVDEMLRSEYGGSFRFSSDAISTTFTTNLTEYPLSRVD
LGDCIGKDARDAMDRIFARRYNATHIKVGQPQYYQANGGFLIAYQPLLSNTLAELYVRE
HLREQSRKPPNPIPPPPGASANASVERIKTTSSIEFARLQFTYNHIQRHVNDMLGRVAIAW
CELQNHELTLWNEARKLNPNAIASVTVGRRVSARMLGDVMAVSTCVPVAADNVIVQNS
MRISSRPGACYSRPLVSFRYEDQGPLVEGQLGENNELRLTRDAIEPCTVGHRRYFTFGGG
YVYFEEYAYSHQLSRADITTVSTFIDLNITMLEDHEFVPLEVYTRHEIKDSGLLDYTEVQR
RNQLHDLRFADIDTVIHADANAAMFAGLGAFFEGMGDLGRAVGKVVMGIVGGVVSAV
SGVSSFMSNPFGALAVGLLVLAGLAAAFFAFRYVMRLQSNPMKALYPLTTKELKNPTNP
DASGEGEEGGDFDEAKLAEAREMIRYMALVSAMERTEHKAKKKGTSALLSAKVTDMV
MRKRRNTNYTQVPNKDGDADEDDL.

In another embodiment, a gB-1 protein utilized in methods and compositions of the present invention is a homologue, isoform or variant of SEQ ID NO: 7. For example, a gB-1 protein may have 95% or greater homology to SEQ ID NO: 7 or may be a variant having up to 20 amino acid changes (any combination of additions, deletions or substitutions). In another embodiment, the gB-1 protein is an immunogenic fragment of any of SEQ ID NO: 7, a homologue of SEQ ID NO: 7, an isoform of SEQ ID NO: 7, or a variant of SEQ ID NO: 7.

In another embodiment, the nucleic acid sequence encoding a gB-1 protein utilized in methods and compositions of the present invention is set forth in a GenBank entry having one of the following Accession Numbers: AB252863, X14112, AF097023, E03024, E03025, E03026, E03092, E03112, E03113, E03114, K01760, K02720, K03541, M14164, or U49121. In another embodiment, the gB-1 protein is encoded by a nucleotide molecule having a sequence set forth in one of the above GenBank entries. In another embodiment, the gB-1 protein is a homologue, isoform or variant of a protein encoded by a sequence set forth in one of the above GenBank entries. In another embodiment, the gB-1 protein is a fragment of any of a protein encoded by a sequence set forth in one of the above GenBank entries, a homologue of a protein encoded by a sequence set forth in one of the above GenBank entries, an isoform of a protein encoded by a sequence set forth in one of the above GenBank entries, or a variant of a protein encoded by a sequence set forth in one of the above GenBank entries.

In another embodiment, the gB-1 fragment consists of about AA 31-659, about AA 31-693, or about AA 31-773.

In certain embodiments, the gB-1 fragment is a fusion between the ectodomain and the cytoplasmic domain, i.e., is an internally deleted fragment. Such fragments may provide optimal secretion of gB. Exemplary fragments of this kind include, but are not limited to, fragments having about AA 31-659 fused to AA 799-904, about AA 31-693 fused to AA 796-904, about AA 31-773 fused to AA 799-904, about AA 31-659 fused to AA 874-904, about AA 31-693 fused to AA 874-904, or about AA 31-773 fused to AA 874-904.

In another embodiment, the gB protein fragment of methods and compositions of the present invention is a gB-2 fragment. In another embodiment, the gB-2 fragment consists of, or alternatively comprises, the gB-2 ectodomain, or a fragment thereof. In another embodiment, the gB-2 fragment is any other gB-2 fragment known in the art.

In one embodiment, the gB-2 protein utilized in methods and compositions of the present invention has, in another embodiment, the sequence:

(SEQ ID NO: 8)
MRGGGLICALVVGALVAAVASAAPAAPAAPRASGGVAATVAANGGPASRPPPVPSPAT
TKARKRKTKKPPKRPEATPPPDANATVAAGHATLRAHLREIKVENADAQFYVCPPPTGA
TVVQFEQPRRCPTRPEGQNYTEGIAVVFKENIAPYKFKATMYYKDVTVSQVWFGHRYS
QFMGIFEDRAPVPFEEVIDKINTKGVCRSTAKYVRNNMETTAFHRDDHETDMELKPAKV
ATRTSRGWHTTDLKYNPSRVEAFHRYGTTVNCIVEEVDARSVYPYDEFVLATGDFVYM
SPFYGYREGSHTEHTSYAADRFKQVDGFYARDLTTKARATSPTTRNLLTTPKFTVAWD
WVPKRPAVCTMTKWQEVDEMLRAEYGGSFRFSSDAISTTFTTNLTEYSLSRVDLGDCIG
RDAREAIDRMFARKYNATHIKVGQPQYYLATGGFLIAYQPLLSNTLAELYVREYMREQD
RKPRNATPAPLREAPSANASVERIKTTSSTEFARLQFTYNHIQRHVNDMLGRIAVAWCEL
QNHELTLWNEARKLNPNAIASATVGRRVSARMLGDVMAVSTCVPVAPDNVIVQNSMR
VSSRPGTCYSRPLVSFRYEDQGPLIEGQLGENNELRLTRDALEPCTVGHRRYFIFGGGYV
YFEEYAYSHQLSRADVTTVSTFIDLNITMLEDHEFVPLEVYTRHEIKDSGLLDYTEVQRR
NQLHDLRFADIDTVIRADANAAMFAGLCAFFEGMGDLGRAVGKVVMGVVGGVVSAVS
GVSSFMSNPFGALAVGLLVLAGLVAAFFAFRYVLQLQRNPMKALYPLTTKELKTSDPGG
VGGEGEEGAEGGGEDEAKLAEAREMIRYMALVSAMERTEHKARKKGTSALLSSKVTN
MVLRKRNKARYSPLHNEDEAGDEDEL.

In another embodiment, a gB-2 protein utilized in methods and compositions of the present invention is a homologue, isoform or variant of SEQ ID NO: 8. For example, a gB-2 protein may have 95% or greater homology to SEQ ID NO: 8 or may be a variant having up to 20 amino acid changes (any combination of additions, deletions or substitutions). In another embodiment, the protein is an immunogenic fragment of any of SEQ ID NO: 8, a homologue of SEQ ID NO: 8, an isoform of SEQ ID NO: 8, or a variant of SEQ ID NO: 8.

In another embodiment, the nucleic acid sequence encoding a gB-2 protein utilized in methods and compositions of the present invention is set forth in a GenBank entry having one of the following Accession Numbers: AAB60546, ABU45428, AF021340, AY630441, BAG49514, HHU12172, HHU12173, HHU12174, HHU12175, M14923, M15118, P24994, or Z86099. In another embodiment, the gB-2 protein is encoded by a nucleotide molecule having a sequence set forth in one of the above GenBank entries. In another embodiment, the protein is a homologue, isoform, or variant of a protein encoded by a sequence set forth in one of the above GenBank entries. In another embodiment, the protein is a fragment of any of a protein encoded by a sequence set forth in one of the above GenBank entries, a homologue of a protein encoded by a sequence set forth in one of the above GenBank entries, an isoform of a protein encoded by a sequence set forth in one of the above GenBank entries, or a variant of a protein encoded by a sequence set forth in one of the above GenBank entries.

In another embodiment, the gB-2 fragment consists of about AA 23-657, about AA 23-720, or about AA 23-748.

In certain embodiments, the gB-2 fragment is a fusion between the ectodomain and the cytoplasmic domain, i.e., is an internally deleted fragment. Such fragments may provide optimal secretion of gB. Exemplary fragments of this kind include, but are not limited to, fragments having about AA 23-657 fused to AA 796-904, about AA 23-720 fused to AA 796-904, about AA 23-748 fused to AA 796-904, about AA 23-657 fused to AA 874-904, about AA 23-720 fused to AA 874-904, or about AA 23-748 fused to AA 874-904.

In another embodiment, the recombinant gB protein, or fragment thereof, elicits antibodies that inhibit binding of gB to a cellular receptor. A cellular receptor is selected from paired immunoglobulin-like type 2 receptor (PILRα) (Satoh et al., 2008, Cell 132:935-944), a heparan sulfate proteoglycan or any other gB receptor known in the art.

In another embodiment, a gB protein fragment utilized in methods and compositions of the present invention is an immunogenic fragment. In another embodiment, mutants, sequence conservative variants, and functional conservative variants of gB are useful in methods and compositions of the present invention, provided that all such variants retain the required immunoprotective effect.

In another embodiment, the immunogenic fragment can comprise an immunoprotective gB antigen from any strain of HSV. In another embodiment, the immunogenic fragment can comprise sequence variants of HSV, as found in infected individuals.

### Analogs, Homologues, Isoforms and Variants

The present invention also provides for analogs of HSV glycoproteins, including any of the above glycoproteins, or fragments thereof. Analogs may differ from naturally occurring proteins or peptides by conservative amino acid sequence substitutions or by modifications which do not affect sequence, or by both.

For example, conservative amino acid changes may be made, which although they alter the primary sequence of the protein or peptide, do not normally alter its function. Conservative amino acid substitutions typically include substitutions within the following groups: (a) glycine, alanine; (b) valine, isoleucine, leucine; (c) aspartic acid, glutamic acid; (d) asparagine, glutamine; (e) serine, threonine; (f) lysine, arginine; (g) phenylalanine, tyrosine.

Modifications (which do not normally alter primary sequence) include in vivo, or in vitro chemical derivatization of polypeptides, e.g., acetylation, or carboxylation. Also included are modifications of glycosylation, e.g., those made by modifying the glycosylation patterns of a polypeptide during its synthesis and processing or in further processing steps; e.g., by exposing the polypeptide to enzymes which affect glycosylation, e.g., mammalian glycosylating or deglycosylating enzymes. Also included are sequences which have phosphorylated amino acid residues, e.g., phosphotyrosine, phosphoserine, or phosphothreonine.

Also included are polypeptides which have been modified using ordinary molecular biological techniques so as to improve their resistance to proteolytic degradation or to optimize solubility properties or to render them more suitable as a therapeutic agent. Analogs of such polypeptides include those containing residues other than naturally occurring L-amino acids, e.g., D-amino acids or non-naturally occurring synthetic amino acids.

It is to be understood that the compositions and methods of the present invention may be used in non-HSV herpesvirus as well, which comprise gD, gE, gB or gC proteins that are 70%, 80%, 85%, 90%, 95%, 98%, or 99% homologous to the gD, gE, gB, or gC proteins of HSV-1 or HSV-2. Such vaccines may be useful in suppressing, inhibiting, preventing, or treating, cancers, or in another embodiment, tumors. Non-HSV herpesvirus include, but are not limited to Varicella Zoster Virus (VZV), Epstein-Barr virus (EBV), EBNA, cytomegalovirus (CMV), and human herpesvirus-6 (HHV-6).

The present invention provides for homologues of HSV glycoproteins, or fragments thereof. The terms “homology,” “homologous,” etc, when in reference to any protein or peptide, refer to a percentage of AA residues in the candidate sequence that are identical with the residues of a corresponding native polypeptide, after aligning the sequences and introducing gaps, if necessary, to achieve the maximum percent homology, and not considering any conservative substitutions as part of the sequence identity. Methods and computer programs for the alignment are well known in the art.

Homology is, in another embodiment, determined by computer algorithm for sequence alignment, by methods well described in the art. For example, computer algorithm analysis of nucleic acid sequence homology can include the utilization of any number of software packages available, such as, for example, the BLAST, DOMAIN, BEAUTY (BLAST Enhanced Alignment Utility), GENPEPT and TREMBL packages.

In another embodiment, “homology” refers to identity to a sequence selected from SEQ ID NO: 1-8 of greater than 70%, greater than 72%, greater than 75%, greater than 78%, greater than 80%, greater than 82%, greater than 83%, greater than 85%, greater than 87%, greater than 88%, greater than 90%, greater than 92%, greater than 93%, greater than 95%, greater than 96%, greater than 97%, greater than 98%, or greater than 99%.

Homology may also be determined via determination of candidate sequence hybridization, methods of which are well described in the art. See, for example, “Nucleic Acid Hybridization” Hames, B. D., and Higgins S. J., Eds. (1985); Sambrook et al., 2001, Molecular Cloning, A Laboratory Manual, Cold Spring Harbor Press, N.Y.; and Ausubel et al., 1989, Current Protocols in Molecular Biology, Green Publishing Associates and Wiley Interscience, N.Y. Methods of hybridization may be carried out under moderate to stringent conditions, to the complement of a DNA encoding a HSV glycoprotein described herein. Hybridization conditions being, for example, overnight incubation at 42° C. in a solution comprising: 10-20 formamide, 5×SSC (150 mM NaCl, 15 mM trisodium citrate), 50 mM sodium phosphate (pH 7.6), 5×Denhardt's solution, 10% dextran sulfate, and 20 μg/ml denatured, sheared salmon sperm DNA.

Protein and/or peptide homology for any AA sequence listed herein may be determined by methods well described in the art, including immunoblot analysis, or via computer algorithm analysis of AA sequences, utilizing any of a number of software packages available, via established methods. Some of these packages include the FASTA, BLAST, MPsrch or Scanps packages, and employ the use of the Smith and Waterman algorithms, and/or global/local or BLOCKS alignments for analysis, for example.

The present invention also provides for variants of HSV glycoproteins, or fragments thereof. As used herein, “variant” refers to an amino acid or nucleic acid sequence (or an organism or tissue) that is different from the majority of the population but is still sufficiently similar to the common mode to be considered to be one of them, for example, splice variants. The variant may be a sequence conservative variant or may be a functional conservative variant. A variant may comprise an addition, deletion or substitution (in any combination) of 1, 2, 3, 4, 5, 7 or 10 amino acids. A variant may also comprise an addition, deletion or substitution, or combination thereof of 2-15, 3-20 or 4-25 amino acids. As used herein, variants includes analogs as defined above.

The present invention also provides for isoforms of HSV glycoproteins, or fragments thereof. As used herein, “isoform” refers to a version of a molecule, for example, a protein, with only slight differences to another isoform of the same protein. Isoforms may be produced from different but related genes or may arise from the same gene by alternative splicing. Isoforms may also be caused by single nucleotide polymorphisms.

In another embodiment, methods and compositions of the present invention utilize a chimeric molecule, comprising a fusion of a recombinant HSV protein with a tag polypeptide that provides an epitope to which an anti-tag antibody can selectively bind. The epitope tag is placed, for example, at the amino- or carboxyl-terminus of the protein or in an internal location therein. The presence of such epitope-tagged forms of the recombinant HSV protein can be detected using an antibody against the tag polypeptide. In another embodiment, inclusion of the epitope tag enables the recombinant HSV protein to be readily purified by affinity purification using an anti-tag antibody or another type of affinity matrix that binds to the epitope tag. Various tag polypeptides and their respective antibodies are known in the art. Examples include poly-histidine (poly-his) or poly-histidine-glycine (poly-his-gly) tags; the flu HA tag polypeptide and its antibody 12CA5 (Field et al., 1988, Mol. Cell. Biol. 8:2159-2165); the c-myc tag and the 8F9, 3C7, 6E10, G4, B7 and 9E10 antibodies thereto (Evan et al., 1985, Mol Cell Biol 5:3610-3616); and the Herpes Simplex virus glycoprotein D (gD) tag and its antibody (Paborsky et al., 1990, Protein Engineering 3:547-553). Other tag polypeptides include the Flag-peptide (Hopp et al., 1988, BioTechnology 6:1204-1210); the KT3 epitope peptide (Martin et al., 1992, Science 255: 192-194); a tubulin epitope peptide (Skinner et al., 1991, J. Biol. Chem. 266:15163-15166); and the T7 gene 10 protein peptide tag (Lutz-Freyermuth et al., 1990, Proc. Natl. Acad. Sci. USA 87: 6393-6397). A preferred His tag for any of the above glycoproteins and fragments is 3 Gly residues prior to 9 His residues, which may be at the N-terminus or C-terminus. In certain embodiments, in particular for gC, when placed at the C-terminus, a Factor Xa recognition sequence (IEGR) has been added to cleave off the His tag. In another embodiment, the chimeric molecule comprises a fusion of the recombinant HSV protein with an immunoglobulin or a particular region of an immunoglobulin. Methods for constructing fusion proteins are well known in the art, and are described, for example, in LaRochelle et al., 1995, J. Cell Biol. 139:357-66; Heidaran et al., 1995, FASEB J. 9:140-5; Ashkenazi et al., 1993, Int. Rev. Immunol. 10:219-27; and Cheon et al., 1994, Proc Natl. Acad Sci USA 91:989-93. In another embodiment, the chimeric molecule comprises a fusion of the recombinant HSV protein with sequences coding for a glycosylphosphatidylinositol linkage. Methods for constructing fusion proteins are well known in the art, and are described in, for example, Kemble et al., 1993, J. Cell Biol. 122:1253-1265; Kemble et al., 1994, Cell 76:383-391; Tong and Comptons, 2000, Virology 270:368-376; Melikyan, 1997, J. Cell Biol. 136:995-1005; Weiss and White, 1993, J. Virol. 67:7060-7066; Zhou et al., 1997, Virology 239:327-339; and Salzwekel et al., 1993, J. Virol. 67:5279-5288.

The sequences can also be modified with signal sequences, including pre-signal sequences. Examples of signal sequences include, but are not limited to, S. cerevisiae alpha factor pre signal sequence, S. cerevisiae alpha factor prepro signal sequence, Aspergillus niger α-amylase signal sequence, human Serum Albumin signal sequence, P. pastoris PHO1 signal sequence, P. pastoris KAR2 signal sequence, Chicken Lysozyme signal sequence, Kluyveromyces lactis Killer toxin pre signal sequence, Aspergillus awamori glucoamylase signal sequence.

### Codon Optimization

The present invention further provides isolated DNA sequences encoding HSV glycoproteins which are codon-optimized for high level expression in yeast. In certain embodiments, the isolated nucleic acids comprise a nucleotide sequence of SEQ ID NO: 10, 12, 14, or 16. In one embodiment, the isolated nucleic acids further comprise a signal sequence and/or a HIS tag.

In certain embodiments, DNA encoding HSV glycoproteins are codon-optimized for high level expression in a yeast cell. In alternative embodiments, the nucleotide sequence is altered to eliminate transcription termination signals that are recognized by yeast.

A “triplet” codon of four possible nucleotide bases can exist in over 60 variant forms. Because these codons provide the message for only 20 different amino acids (as well as transcription initiation and termination), some amino acids can be coded for by more than one codon, a phenomenon known as codon redundancy. There appears to exist a variable natural hierarchy or “preference” for certain codons in certain types of cells. As one example, the amino acid leucine is specified by any of six DNA codons including CTA, CTC, CTG, CTT, TTA, and TTG. Exhaustive analysis of genome codon use frequencies for microorganisms has revealed endogenous DNA of E. coli most commonly contains the CTG leucine-specifying codon, while the DNA of yeasts and slime molds most commonly includes a TTA leucine-specifying codon. In view of this hierarchy, it is generally believed that the likelihood of obtaining high levels of expression of a leucine-rich polypeptide by an E. coli host will depend to some extent on the frequency of codon use. For example, it is likely that a gene rich in TTA codons will be poorly expressed in E. coli, whereas a CTG rich gene will probably be highly expressed in this host. Similarly, a preferred codon for expression of a leucine-rich polypeptide in yeast host cells would be TTA.

The phenomenon of codon preference phenomena on recombinant DNA techniques may serve to explain many prior failures to achieve high expression levels of exogenous genes in successfully transformed host organisms—a less “preferred” codon may be repeatedly present in the inserted gene and the host cell machinery for expression may not operate as efficiently. This phenomenon suggests that synthetic genes which have been designed to include a projected host cell's preferred codons provide an optimal form of foreign genetic material for practice of recombinant protein expression. In a preferred embodiment of this invention, it has been found that the use of alternative codons encoding the same protein sequence may remove the constraints on expression of HSV glycoproteins by yeast cells.

In accordance with this invention, HSV glycoprotein gene segments were converted to sequences having identical translated sequences but with alternative codon usage as described by Sharp and Cowe for S. cerevisiae (Sharp et al., 1991, Yeast 7: 657-678) or Sinclair and Choy for Pichia pastoris (Sinclair et al., 2002, Protein Expr Purif 26:96-105). See Table 1 (from Sinclair and Choy).

TABLE 1
Preferred codons from P. pastoris and S. cerevisaie

| Amino acid | P. pastoris codon | S. cerevisiae codon |
|---|---|---|
| Phe | TTC | TTC |
| Leu | TTG | TTG |
| Ile | ATC | ATC/ATT |
| Val | GTT/GTC | GTT/GTC |
| Tyr | TAG | TAG |
| His | CAC | CAC |
| Asn | AAC | AAC |
| Lys | AAG | AAG |
| Asp | GAC | GAC |
| Glu | GAG | GAG |
| Ser | TCT/TCC | TCT/TCC |
| Pro | CCA | CCA |
| Thr | ACC | ACC/ACT |
| Ala | GCT/GCC | GCT |
| Arg | AGA | AGA |
| Gly | GGT | GGT |
| Gln | CAA/CAG | CAA |
| Cys | TGT/TGC | TGT |

The methodology generally consists of identifying codons in the wild-type sequence that are not commonly associated with highly expressed yeast genes and replacing them with optimal codons for high expression in yeast cells. Thus, the DNA encoding an HSV glycoprotein can be altered (using site-directed mutagenesis or any other method known in the art) such that one or more codons for Phe, Len, Ile, Val, Tyr, His, Asn, Lys, Asp, Glu, Ser, Pro, Thr, Ala, Arg, Gly, Gln, or Cys are changed to a preferred yeast codon as shown in Table 1. For each amino acid, one, two, three, four, five, up to all of the codons for a particular amino acid can be changed to the preferred codon. The new gene sequence is then inspected for undesired sequences generated by these codon replacements (e.g., “ATTTA” sequences, inadvertent creation of intron splice recognition sites, unwanted restriction enzyme sites, high GC content, presence of transcription termination signals that are recognized by yeast, etc.). Undesirable sequences are eliminated by substitution of the existing codons with different codons coding for the same amino acid. The synthetic gene segments are then tested for improved expression.

The methods described above were used to create synthetic gene segments for HSV glycoproteins, resulting in a gene comprising codons optimized for high-level expression. See Table 2. While the above procedure provides a summary of one methodology for designing codon-optimized genes for use in HSV vaccines, it is understood by one skilled in the art that similar vaccine efficacy or increased expression of genes may be achieved by minor variations in the procedure or by minor variations in the sequence.

TABLE 2
Codon optimized HSV glycoprotein sequences

| | | SEQ ID NO: |
|---|---|---|
| | | (without |
| | | signal sequence |
| HSV | | and HIS tag and |
| glycoprotein | Nucleotide sequence | stop) |
| HSV-2 gD 339 | atgagatttccttcaatttttactgctgttttattcgc | SEQ ID NO: 10 |
| | agcatcctccgcattagctAAGTACGCTTTGGCTGACC | |
| | CATCTTTGAAGATGGCTGACCCAAACAGATTCAGAGGA | |
| | AAGAACTTGCCAGTTTTGGACCAATTGACTGACCCACC | |
| | AGGTGTTAAGAGAGTTTACCACATCCAACCATCTTTGG | |
| | AGGATCCATTCCAACCACCATCCATCCCAATCACTGTT | |
| | TACTACGCTGTTTTGGAGAGAGCTTGTAGATCCGTTTT | |
| | GTTGCACGCTCCATCTGAAGCTCCACAGATTGTTAGAG | |
| | GAGCTTCTGACGAAGCTAGAAAGCACACTTACAACTTG | |
| | ACTATCGCTTGGTACAGAATGGGTGACAACTGTGCTAT | |
| | CCCTATCACTGTTATGGAATACACTGAGTGTCCATACA | |
| | ACAAGTCCTTGGGAGTTTGTCCAATCAGAACTCAGCCA | |
| | AGATGGTCCTACTACGATTCCTTCTCTGCTGTTTCCGA | |
| | GGATAACTTGGGATTCTTGATGCACGCTCCAGCTTTTG | |
| | AAACTGCTGGTACTTACTTGAGATTGGTTAAGATCAAC | |
| | GACTGGACTGAGATCACTCAGTTCATCTTGGAGCACAG | |
| | AGCTAGAGCTTCATGTAAGTATGCTTTGCCATTGAGAA | |
| | TCCCACCTGCTGCTTGTTTGACTTCCAAGGCTTACCAA | |
| | CAGGGTGTTACTGTTGATTCCATCGGAATGTTGCCAAG | |
| | ATTCATCCCAGAGAACCAGAGAACTGTTGCTTTGTACT | |
| | CCTTGAAGATCGCTGGATGGCATGGTCCAAAGCCACCA | |
| | TACACTTCCACTTTGTTGCCACCAGAATTGTCCGACAC | |
| | TACAAACGCTACTCAGCCAGAATTGGTTCCAGAAGATC | |
| | CAGAGGATTCCGCTTTGTTGGAAGATCCAGCTGGTACT | |
| | GTTTCCTCCCAAATCCCACCAAACTGGCACATTCCATC | |
| | CATTCAAGACGTTGCTCCACATCATGCTCCAGCTGCTC | |
| | CATCTAATCCAGGTGGTGGTCATCATCACCATCACCAC | |
| | CACCATCACTAATAG | |
| | (SEQ ID NO: 9) | |
| | Lower case is the S. cerevisiae | |
| | alpha factor pre signal sequence/ | |
| | Underlined is the G3H9 His tag | |
| | and stop | |
| HSV-2 gD 310 | atgagatttccttcaatttttactgctgttttattcgc | SEQ ID NO: 12 |
| | agcatcctccgcattagctAAGTACGCTTTGGCTGACC | |
| | CATCTTTGAAGATGGCTGACCCAAACAGATTCAGAGGA | |
| | AAGAACTTGCCAGTTTTGGACCAATTGACTGACCCACC | |
| | AGGTGTTAAGAGAGTTTACCACATCCAACCATCTTTGG | |
| | AGGATCCATTCCAACCACCATCCATCCCAATCACTGTT | |
| | TACTACGCTGTTTTGGAGAGAGCTTGTAGATCCGTTTT | |
| | GTTGCACGCTCCATCTGAAGCTCCACAGATTGTTAGAG | |
| | GAGCTTCTGACGAAGCTAGAAAGCACACTTACAACTTG | |
| | ACTATCGCTTGGTACAGAATGGGTGACAACTGTGCTAT | |
| | CCCTATCACTGTTATGGAATACACTGAGTGTCCATACA | |
| | ACAAGTCCTTGGGAGTTTGTCCAATCAGAACTCAGCCA | |
| | AGATGGTCCTACTACGATTCCTTCTCTGCTGTTTCCGA | |
| | GGATAACTTGGGATTCTTGATGCACGCTCCAGCTTTTG | |
| | AAACTGCTGGTACTTACTTGAGATTGGTTAAGATCAAC | |
| | GACTGGACTGAGATCACTCAGTTCATCTTGGAGCACAG | |
| | AGCTAGAGCTTCATGTAAGTATGCTTTGCCATTGAGAA | |
| | TCCCACCTGCTGCTTGTTTGACTTCCAAGGCTTACCAA | |
| | CAGGGTGTTACTGTTGATTCCATCGGAATGTTGCCAAG | |
| | ATTCATCCCAGAGAACCAGAGAACTGTTGCTTTGTACT | |
| | CCTTGAAGATCGCTGGATGGCATGGTCCAAAGCCACCA | |
| | TACACTTCCACTTTGTTGCCACCAGAATTGTCCGACAC | |
| | TACAAACGCTACTCAGCCAGAATTGGTTCCAGAAGATC | |
| | CAGAGGATTCCGCTTTGTTGGAAGATCCAGCTGGTACT | |
| | GGTGGTGGTCATCATCACCATCACCACCACCATCACTA | |
| | ATAG | |
| | (SEQ ID NO: 11) | |
| | Lower case is the S. cerevisiae | |
| | alpha factor pre signal sequence/ | |
| | Underlined is the G3H9 His tag | |
| | and stop | |
| HSV-2 gC | atgagatttccttcaatttttactgctgttttattcgc | SEQ ID NO: 14 |
| Full length | agcatcctccgcattagctgctccagtcaacactacaa | |
| ectodomain | cagaagatgaaacggcacaaattccggctgaagctgtc | |
| | atcggttactcagatttagaaggggatttcgatgttgc | |
| | tgttttgccattttccaacagcacaaataacgggttat | |
| | tgtttataaatactactattgccagcattgctgctaaa | |
| | gaagaaggggtatctctcgagaaaagaATGGGTCATCA | |
| | TCATCACCATCACCACCATCACGGTGGTGGTATCGAAG | |
| | GTAGATTGGCTAATGCTTCTCCAGGTAGAACTATCACT | |
| | GTTGGTCCAAGAGGTAACGCTTCTAACGCTGCTCCATC | |
| | TGCTTCTCCAAGAAACGCTTCCGCTCCAAGAACTACTC | |
| | CAACTCCACCACAACCAAGAAAGGCTACTAAGTCCAAG | |
| | GCTTCCACTGCTAAACCAGCTCCACCACCAAAAACTGG | |
| | TCCACCAAAGACTTCCTCTGAGCCAGTTAGATGTAACA | |
| | GACACGACCCATTGGCTAGATACGGTTCCAGAGTTCAG | |
| | ATCAGATGTAGATTCCCAAACTCCACTAGAACTGAGTT | |
| | CAGATTGCAGATTTGGAGATACGCTACTGCTACTGACG | |
| | CTGAGATTGGTACTGCTCCATCCTTGGAAGAGGTTATG | |
| | GTTAACGTTTCTGCTCCACCAGGTGGTCAATTGGTTTA | |
| | CGATTCCGCTCCTAACAGAACTGACCCACACGTTATTT | |
| | GGGCTGAAGGTGCTGGTCCTGGTGCTTCTCCTAGATTG | |
| | TACTCCGTTGTTGGTCCATTGGGTAGACAGAGATTGAT | |
| | CATCGAAGAGTTGACTTTGGAGACACAGGGAATGTACT | |
| | ACTGGGTCTGGGGTAGAACTGATAGACCATCCGCTTAC | |
| | GGTACTTGGGTCCGTGTTAGAGTTTTCAGACCACCATC | |
| | CTTGACTATTCACCCACACGCTGTTTTGGAAGGACAAC | |
| | CATTCAAGGCTACTTGTACTGCTGCTACTTACTACCCA | |
| | GGTAACAGAGCTGAGTTCGTCTGGTTCGAGGACGGTAG | |
| | AAGAGTTTTCGACCCAGCTCAAATCCACACTCAGACAC | |
| | AAGAAAACCCAGACGGTTTCTCCACTGTTTCCACAGTT | |
| | ACTTCCGCTGCTGTTGGTGGTCAAGGTCCACCAAGAAC | |
| | TTTCACTTGTCAGTTGACTTGGCACAGAGATTCTGTTT | |
| | CCTTCTCCAGAAGAAATGCTTCTGGTACTGCTTCCGTT | |
| | TTGCCAAGACCAACTATCACTATGGAGTTCACTGGTGA | |
| | CCATGCTGTTTGCACTGCTGGTTGTGTTCCAGAGGGTG | |
| | TTACTTTCGCTTGGTTTTTGGGTGACGATTCTTCTCCA | |
| | GCTGAGAAAGTTGCTGTTGCTTCCCAAACTTCCTGTGG | |
| | TAGACCAGGTACTGCTACTATCAGATCCACTTTGCCAG | |
| | TTTCCTACGAGCAAACTGAGTACATCTGTAGATTGGCT | |
| | GGTTACCCAGACGGAATCCCAGTTTTGGAACACCACGG | |
| | TTCTCATCAACCACCACCAAGAGACCCAACTGAGAGAC | |
| | AGGTTATCAGAGCTATCGAAGGTAGATAATAA | |
| | (SEQ ID NO: 13) | |
| | Lower case is the S. cerevisiae | |
| | alpha factor pre pro signal | |
| | sequence/Underlined is the G3H9 | |
| | His tag and stop | |
| HSV-2 gC N- | atgagatttccttcaatttttactgctgttttattcgc | SEQ ID NO: 16 |
| terminally | agcatcctccgcattagctgctccagtcaacactacaa | |
| truncated | cagaagatgaaacggcacaaattccggatgaagctgtc | |
| ectodomain | atcggttactcagatttagaaggggatttcgatgttgc | |
| (starting | tgttttgccattttccaacagcacaaataacgggttat | |
| TAKPA) | tgtttataaatactactattgccagcattgctgctaaa | |
| | gaagaaggggtatctatcgagaaaagaCATCATCATCA | |
| | CCATCACCACCATCACGGTGGTGGTACTGCTAAACCAG | |
| | CTCCACCACCAAAAACTGGTCCACCAAAGACTTCCTCT | |
| | GAGCCAGTTAGATGTAACAGACACGACCCATTGGCTAG | |
| | ATACGGTTCCAGAGTTCAGATCAGATGTAGATTCCCAA | |
| | ACTCCACTAGAACTGAGTTCAGATTGCAGATTTGGAGA | |
| | TACGCTACTGCTACTGACGCTGAGATTGGTACTGCTCC | |
| | ATCCTTGGAAGAGGTTATGGTTAACGTTTCTGCTCCAC | |
| | CAGGTGGTCAATTGGTTTACGATTCCGCTCCTAACAGA | |
| | ACTGACCCACACGTTATTTGGGCTGAAGGTGCTGGTCC | |
| | TGGTGCTTCTCCTAGATTGTACTCCGTTGTTGGTCCAT | |
| | TGGGTAGACAGAGATTGATCATCGAAGAGTTGACTTTG | |
| | GAGACACAGGGAATGTACTACTGGGTCTGGGGTAGAAC | |
| | TGATAGACCATCCGCTTACGGTACTTGGGTCCGTGTTA | |
| | GAGTTTTCAGACCACCATCCTTGACTATTCACCCACAC | |
| | GCTGTTTTGGAAGGACAACCATTCAAGGCTACTTGTAC | |
| | TGCTGCTACTTACTACCCAGGTAACAGAGCTGAGTTCG | |
| | TCTGGTTCGAGGACGGTAGAAGAGTTTTCGACCCAGCT | |
| | CAAATCCACACTCAGACACAAGAAAACCCAGACGGTTT | |
| | CTCCACTGTTTCCACAGTTACTTCCGCTGCTGTTGGTG | |
| | GTCAAGGTCCACCAAGAACTTTCACTTGTCAGTTGACT | |
| | TGGCACAGAGATTCTGTTTCCTTCTCCAGAAGAAATGC | |
| | TTCTGGTACTGCTTCCGTTTTGCCAAGACCAACTATCA | |
| | CTATGGAGTTCACTGGTGACCATGCTGTTTGCACTGCT | |
| | GGTTGTGTTCCAGAGGGTGTTACTTTCGCTTGGTTTTT | |
| | GGGTGACGATTCTTCTCCAGCTGAGAAAGTTGCTGTTG | |
| | CTTCCCAAACTTCCTGTGGTAGACCAGGTACTGCTACT | |
| | ATCAGATCCACTTTGCCAGTTTCCTACGAGCAAACTGA | |
| | GTACATCTGTAGATTGGCTGGTTACCCAGACGGAATCC | |
| | CAGTTTTGGAACACCACGGTTCTCATCAACCACCACCA | |
| | AGAGACCCAACTGAGAGACAGGTTATCAGAGCTATCGA | |
| | AGGTAGATAATAA | |
| | (SEQ ID NO: 15) | |
| | Lower case is the S. cerevisiae | |
| | alpha factor pre pro signal | |
| | sequence/Underlined is the G3H9 | |
| | His tag and stop | |

### Expression in Pichia Pastoris

The present invention further provides methods for the production of a recombinant Herpes Simplex Virus glycoprotein selected from the group consisting of gC, gD, gB and gE comprising: a) transforming Pichia pastoris with an expression vector containing a DNA encoding an HSV protein selected from the group consisting of gC, gD, gB and gE, which is under regulation of a promoter functional in Pichia pastoris; b) culturing said Pichia pastoris in a medium; and d) recovering/isolating the HSV protein from the cultured Pichia pastoris. In certain embodiments, the glycoprotein is selected from gC, gB and gE. In certain embodiments, the Pichia pastoris is a strain in which one or more of the following modifications have been made: i) disruption of the yeast protein disulfide isomerase gene (PDI); or ii) expression of one or more chaperones selected from the group consisting of human PDI, human calreticulin (hCRT), human Erp57, human FLC1, human ERO1, human FAD1. In certain embodiments, the Pichia pastoris is a glycoengineered Pichia pastoris strain selected from 2.0, 5.9 and 6.0, which optionally has one or more of the above modifications.

A recombinant vector comprising a nucleotide molecule encoding an HSV glycoprotein is utilized for expression in Pichia. The expression vector to be used is not particularly limited as long as it can be maintained stably by autonomous replication in a fungus body of yeast of the genus Pichia or integration into a yeast genome. Examples of the autonomously-replicable vector include YEp vector, YRp vector, YCp vector and the like. In addition, examples of the vector to be integrated into a yeast genome include YIp vector, YRp vector, KINKO vector (see U.S. Pat. No. 7,029,872), and yeast roll-in vector.

A nucleotide encoding an HSV glycoprotein may be operably linked to a promoter/regulatory sequence that drive expression of the encoded peptide in cells into which the vector is introduced. Promoter/regulatory sequences useful for driving constitutive expression of a gene in yeast are well known in the art. Inducible expression of the nucleic acid encoding a HSV glycoprotein may be accomplished by placing the nucleic acid encoding the peptide under the control of an inducible promoter/regulatory sequence. It will be appreciated that the invention includes the use of any promoter/regulatory sequence, which is either known or unknown, and which is capable of driving expression of the desired protein operably linked thereto.

Examples of promoters functional in the yeast of the genus Pichia include promoters derived from a yeast, such as PHO5 promoter, PGK promoter, GAP promoter, ADH promoter derived from S. cerevisiae and the like, alcohol oxidase (AOX) 1 promoter, AOX2 promoter, dihydroxyacetone synthase promoter, P40 promoter, ADH promoter, folic acid dehydrogenase promoter derived from P. pastoris and the like. In addition, the above-mentioned promoter derived from a yeast may be a mutant promoter modified to further improve the gene expression efficiency, for example, mutant AOX2 (mAOX2) promoter (Ohi et al., 1994, Mol. Gen. Genet. 243:489-499; Japanese Patent Application Publication No. JP-A-4-299984) and the like. Preferably, the promoter is a promoter of an enzyme gene necessary for treating methanol or a metabolic intermediate thereof, in order to use a methanol-metabolizing system in the yeast of the genus Pichia, such as AOX1 promoter, mAOX2 promoter and the like, more preferably AOX1 promoter. In another embodiment, the transcription and translation initiation region is the promoter selected from the group consisting of Pichia pastoris formaldehyde dehydrogenase (FLD1), peroxisomal matrix protein (PEX8), and the GTPase encoding YPT1 promoters. In yet another embodiment, a signal peptide sequence such as a signal peptide sequence for a Saccharomyces cerevisiae α-factor is present.

The expression vector containing the DNA encoding an HSV glycoprotein preferably further contains transcription terminator sequence (terminator) functional in a yeast of the genus Pichia (e.g., AOX1 terminator etc.), enhancer sequence, selection marker gene usable for selecting yeast (auxotrophic gene, for example, URA5, HIS4, LEU2, ARG4 and URA3 gene derived from P. pastoris or S. cerevisiae, and the like, or antibiotic resistance gene, for example, resistance gene to Zeocin, cycloheximide, G-418, chloramphenicol, bleomycin, hygromycin, nourseothricin, arsenite, etc., and the like) and the like, and when desired, may contain replicable unit functional in yeast. For integration into the yeast genome the vector more preferably contains a sequence specific to the yeast species, for example, P. pastoris TRP2, URA6, AOX1, and AOX2. For preparation of the vector in a large amount, moreover, the vector more preferably contains a replicable unit functional in Escherichia coli and a selection marker gene usable for selecting Escherichia coli (e.g., resistance gene to ampicillin and tetracycline etc.).

Methods for constructing and utilizing recombinant vectors are well known in the art and are described, for example, in Sambrook et al. (2001, Molecular Cloning: A Laboratory Manual, Cold Spring Harbor Laboratory, New York), and in Brent et al. (2003, Current Protocols in Molecular Biology, John Wiley & Sons, New York).

### Host Cells and Fermentation

Host cells useful in the present invention include Pichia pastoris, Pichia finlandica, Pichia trehalophila, Pichia koclamae, Pichia membranaefaciens, Pichia minuta (Ogataea minuta, Pichia lindneri), Pichia opuntiae, Pichia thermotolerans, Pichia salictaria, Pichia guercuum, Pichia pijperi, Pichia stiptis, Pichia methanolica, Pichia sp., Saccharomyces cerevisiae, Saccharomyces sp., Hansenula polymorpha, Kluyveromyces sp., Kluyveromyces lactis, Candida albicans, Aspergillus nidulans, Aspergillus niger, Aspergillus oryzae, Trichoderma reesei, Chrysosporium lucknowense, Fusarium sp., Fusarium gramineum, Fusarium venenatum and Neurospora crassa. Various yeasts, such as K. lactis, Pichia pastoris, Pichia methanolica, and Hansenula polymorpha are particularly suitable for cell culture because they are able to grow to high cell densities and secrete large quantities of recombinant protein. Likewise, filamentous fungi, such as Aspergillus niger, Fusarium sp, Neurospora crassa and others can be used to produce glycoproteins at an industrial scale.

Lower eukaryotes, particularly yeast and filamentous fungi, can be genetically modified so that they express glycoproteins in which the glycosylation pattern is human-like or humanized. In this manner, glycoprotein compositions can be produced in which a specific desired glycoform is predominant in the composition. Such can be achieved by eliminating selected endogenous glycosylation enzymes and/or genetically engineering the host cells and/or supplying exogenous enzymes to mimic all or part of the mammalian glycosylation pathway as described in U.S. Patent Application Publication No. 2004/0018590 and U.S. Pat. No. 7,029,872, the disclosures of which are hereby incorporated herein by reference. For example, a host cell can be selected or engineered to be depleted in 1,6-mannosyl transferase activities, which would otherwise add mannose residues onto the N-glycan on a glycoprotein. If desired, additional genetic engineering of the glycosylation can be performed, such that the glycoprotein can be produced with or without core fucosylation. Use of lower eukaryotic host cells is further advantageous in that these cells are able to produce highly homogenous compositions of glycoprotein, such that the predominant glycoform of the glycoprotein may be present as greater than thirty mole percent of the glycoprotein in the composition. In particular aspects, the predominant glycoform may be present in greater than forty mole percent, fifty mole percent, sixty mole percent, seventy mole percent and, most preferably, greater than eighty mole percent of the glycoprotein present in the composition.

Using the methods and materials of the present invention, it is possible to produce glycoprotein compositions comprising a plurality of glycoforms, each glycoform comprising at least one N-glycan attached thereto, wherein the glycoprotein composition thereby comprises a plurality of N-glycans in which a predominant glycoform comprises a desired N-glycan. Utilizing the tools described in U.S. Patent Application Publication No. 20040018590 and U.S. Pat. No. 7,029,872, it is possible to produce many different N-linked glycoforms. Depending upon the specific needs, the methods of the present invention can be used to obtain glycoprotein composition in which the predominant N-glycoform is present in an amount between 5 and 80 mole percent greater than the next most predominant N-glycoform; in preferred embodiments, the predominant N-glycoform may be present in an amount between 10 and 40 mole percent; 20 and 50 mole percent; 30 and 60 mole percent; 40 and 70 mole percent; 50 and 80 mole percent greater than the next most predominant N-glycoform. In other preferred embodiments, the predominant N-glycoform is a desired N-glycoform and is present in an amount of greater than 25 mole percent; greater than 35 mole percent; greater than 50 mole percent; greater than 55%; greater than 60 mole percent; greater than 75 mole percent; or greater than 80 mole percent of the total number of N-glycans.

In one embodiment, the host cell further includes an α1,2-mannosidase catalytic domain fused to a cellular targeting signal peptide not normally associated with the catalytic domain and selected to target the α1,2-mannosidase activity to the ER or Golgi apparatus of the host cell. Passage of a recombinant glycoprotein through the ER or Golgi apparatus of the host cell produces a recombinant glycoprotein comprising a Man₅GlcNAc₂ glycoform, for example, a recombinant glycoprotein composition comprising predominantly a Man₅GlcNAc₂ glycoform. For example, U.S. Pat. No. 7,029,872 and U.S. Patent Application Publication Nos. 2004/0018590 and 2005/0170452 disclose lower eukaryote host cells capable of producing a glycoprotein comprising a Man₅GlcNAc₂ glycoform.

In a further embodiment, the immediately preceding host cell further includes a GlcNAc transferase I (GnT I) catalytic domain fused to a cellular targeting signal peptide not normally associated with the catalytic domain and selected to target GlcNAc transferase I activity to the ER or Golgi apparatus of the host cell. Passage of the recombinant glycoprotein through the ER or Golgi apparatus of the host cell produces a recombinant glycoprotein comprising a GlcNAcMan₅GlcNAc₂ glycoform, for example, a recombinant glycoprotein composition comprising predominantly a GlcNAcMan₅GlcNAc₂ glycoform. U.S. Pat. No. 7,029,872 and U.S. Published Patent Application Nos. 2004/0018590 and 2005/0170452 disclose lower eukaryote host cells capable of producing a glycoprotein comprising a GlcNAcMan₅GlcNAc₂ glycoform. The glycoprotein produced in the above cells can be treated in vitro with a hexaminidase to produce a recombinant glycoprotein comprising a Man₅GlcNAc₂ glycoform.

In a further embodiment, the immediately preceding host cell further includes a mannosidase II catalytic domain fused to a cellular targeting signal peptide not normally associated with the catalytic domain and selected to target mannosidase II activity to the ER or Golgi apparatus of the host cell. Passage of the recombinant glycoprotein through the ER or Golgi apparatus of the host cell produces a recombinant glycoprotein comprising a GlcNAcMan₃GlcNAc₂ glycoform, for example a recombinant glycoprotein composition comprising predominantly a GlcNAcMan₃GlcNAc₂ glycoform. U.S. Pat. No. 7,029,872 and U.S. Patent Application Publication No. 2004/0230042 discloses lower eukaryote host cells that express mannosidase II enzymes and are capable of producing glycoproteins having predominantly a GlcNAc₂Man₃GlcNAc₂ glycoform. The glycoprotein produced in the above cells can be treated in vitro with a hexaminidase to produce a recombinant glycoprotein comprising a Man₃GlcNAc₂ glycoform.

In a further embodiment, the immediately preceding host cell further includes GlcNAc transferase II (GnT II) catalytic domain fused to a cellular targeting signal peptide not normally associated with the catalytic domain and selected to target GlcNAc transferase II activity to the ER or Golgi apparatus of the host cell. Passage of the recombinant glycoprotein through the ER or Golgi apparatus of the host cell produces a recombinant glycoprotein comprising a GlcNAc₂Man₃GlcNAc₂ glycoform, for example a recombinant glycoprotein composition comprising predominantly a GlcNAc₂Man₃GlcNAc₂ glycoform. U.S. Pat. No. 7,029,872 and U.S. Patent Application Publication Nos. 2004/0018590 and 2005/0170452 disclose lower eukaryote host cells capable of producing a glycoprotein comprising a GlcNAc₂Man₃GlcNAc₂ glycoform. The glycoprotein produced in the above cells can be treated in vitro with a hexaminidase to produce a recombinant glycoprotein comprising a Man₃GlcNAc₂ glycoform.

In a further embodiment, the immediately preceding host cell further includes a galactosyltransferase catalytic domain fused to a cellular targeting signal peptide not normally associated with the catalytic domain and selected to target galactosyltransferase activity to the ER or Golgi apparatus of the host cell. Passage of the recombinant glycoprotein through the ER or Golgi apparatus of the host cell produces a recombinant glycoprotein comprising a GalGlcNAc₂Man₃GlcNAc₂ or Gal₂GlcNAc₂Man₃GlcNAc₂ glycoform, or mixture thereof, for example, a recombinant glycoprotein composition comprising predominantly a GalGlcNAc₂Man₃GlcNAc₂ glycoform or Gal₂GlcNAc₂Man₃GlcNAc₂ glycoform or mixture thereof. U.S. Pat. No. 7,029,872 and U.S. Patent Application Publication No. 2006/0040353 discloses lower eukaryote host cells capable of producing a glycoprotein comprising a Gal₂GlcNAc₂Man₃GlcNAc₂ glycoform. The glycoprotein produced in the above cells can be treated in vitro with a galactosidase to produce a recombinant glycoprotein comprising a GlcNAc₂Man₃GlcNAc₂ glycoform, for example, a recombinant glycoprotein composition comprising predominantly a GlcNAc₂Man₃GlcNAc₂ glycoform.

In a further embodiment, the immediately preceding host cell further includes a sialyltransferase catalytic domain fused to a cellular targeting signal peptide not normally associated with the catalytic domain and selected to target sialyltransferase activity to the ER or Golgi apparatus of the host cell. Passage of the recombinant glycoprotein through the ER or Golgi apparatus of the host cell produces a recombinant glycoprotein comprising predominantly a NANA₂Gal₂GlcNAc₂Man₃GlcNAc₂ glycoform or NANAGal₂GlcNAc₂Man₃GlcNAc₂ glycoform or mixture thereof. For lower eukaryote host cells such as yeast and filamentous fungi, it is useful that the host cell further include a means for providing CMP-sialic acid for transfer to the N-glycan. U.S. Patent Application Publication No. 2005/0260729 discloses a method for genetically engineering lower eukaryotes to have a CMP-sialic acid synthesis pathway and U.S. Patent Application Publication No. 2006/0286637 discloses a method for genetically engineering lower eukaryotes to produce sialylated glycoproteins. The glycoprotein produced in the above cells can be treated in vitro with a neuraminidase to produce a recombinant glycoprotein comprising predominantly a Gal₂GlcNAc₂Man₃GlcNAc₂ glycoform or GalGlcNAc₂Man₃GlcNAc₂ glycoform or mixture thereof.

Any one of the preceding host cells can further include one or more GlcNAc transferase selected from the group consisting of GnT III, GnT IV, GnT V, GnT VI, and GnT IX to produce glycoproteins having bisected (GnT III) and/or multiantennary (GnT IV, V, VI, and IX) N-glycan structures such as disclosed in U.S. Patent Application Publication Nos. 2004/074458 and 2007/0037248.

In further embodiments, the host cell that produces glycoproteins that have predominantly GlcNAcMan₅GlcNAc₂N-glycans further includes a galactosyltransferase catalytic domain fused to a cellular targeting signal peptide not normally associated with the catalytic domain and selected to target Galactosyltransferase activity to the ER or Golgi apparatus of the host cell. Passage of the recombinant glycoprotein through the ER or Golgi apparatus of the'host cell produces a recombinant glycoprotein comprising predominantly the GalGlcNAcMan₅GlcNAc₂ glycoform.

In a further embodiment, the immediately preceding host cell that produced glycoproteins that have predominantly the GalGlcNAcMan₅GlcNAc₂ N-glycans further includes a sialyltransferase catalytic domain fused to a cellular targeting signal peptide not normally associated with the catalytic domain and selected to target sialyltransferase activity to the ER or Golgi apparatus of the host cell. Passage of the recombinant glycoprotein through the ER or Golgi apparatus of the host cell produces a recombinant glycoprotein comprising a NANAGalGlcNAcMan₅GlcNAc₂ glycoform.

Various of the preceding host cells further include one or more sugar transporters such as UDP-GlcNAc transporters (for example, Kluyveromyces lactic and Mus musculus UDP-GlcNAc transporters), UDP-galactose transporters (for example, Drosophila melanogaster UDP-galactose transporter), and CMP-sialic acid transporter (for example, human sialic acid transporter). Because lower eukaryote host cells such as yeast and filamentous fungi lack the above transporters, it is preferable that lower eukaryote host cells such as yeast and filamentous fungi be genetically engineered to include the above transporters.

Host cells further include lower eukaryote cells (e.g., yeast such as Pichia pastoris) that are genetically engineered to eliminate glycoproteins having α-mannosidase-resistant N-glycans by deleting or disrupting one or more of the β-mannosyltransferase genes (e.g., BMT1, BMT2, BMT3, and BMT4)(See U.S. Patent Application Publication No. 2006/0211085) and glycoproteins having phosphomannose residues by deleting or disrupting one or both of the phosphomannosyl transferase genes PNO1 and MNN4B (See, e.g., U.S. Pat. Nos. 7,198,921 and 7,259,007), which in further aspects can also include deleting or disrupting the MNN4A gene. Disruption includes disrupting the open reading frame encoding the particular enzymes or disrupting expression of the open reading frame or abrogating translation of RNAs encoding one or more of the β-mannosyltransferases and/or phosphomannosyltransferases using interfering RNA, antisense RNA, or the like. The host cells can further include any one of the aforementioned host cells modified to produce particular N-glycan structures.

Host cells further include lower eukaryote cells (e.g., yeast such as Pichia pastoris) that are genetically modified to control O-glycosylation of the glycoprotein by deleting or disrupting one or more of the protein O-mannosyltransferase (Dol-P-Man:Protein (Ser/Thr) Mannosyl Transferase genes) (PMTs) (See U.S. Pat. No. 5,714,377) or grown in the presence of Pmtp inhibitors and/or an alpha-mannosidase as disclosed in PCT International Application Publication No. WO 2007/061631, or both. Disruption includes disrupting the open reading frame encoding the Pmtp or disrupting expression of the open reading frame or abrogating translation of RNAs encoding one or more of the Pmtps using interfering RNA, antisense RNA, or the like. The host cells can further include any one of the aforementioned host cells modified to produce particular N-glycan structures.

Pmtp inhibitors include but are not limited to a benzylidene thiazolidinediones. Examples of benzylidene thiazolidinediones that can be used are 5-[[3,4-bis(phenylmethoxy) phenyl]methylene]-4-oxo-2-thioxo-3-thiazolidineacetic Acid; 5-[[3-(1-Phenylethoxy)-4-(2-phenylethoxy)]phenyl]methylene]-4-oxo-2-thioxo-3-thiazolidineacetic Acid; and 5-[[3-(1-Phenyl-2-hydroxy)ethoxy)-4-(2-phenylethoxy)]phenyl]methylene]-4-oxo-2-thioxo-3-thiazolidineacetic Acid.

In particular embodiments, the function or expression of at least one endogenous PMT gene is reduced, disrupted, or deleted. For example, in particular embodiments the function or expression of at least one endogenous PMT gene selected from the group consisting of the PMT1, PMT2, PMT3, and PMT4 genes is reduced, disrupted, or deleted; or the host cells are cultivated in the presence of one or more PMT inhibitors. In further embodiments, the host cells include one or more PMT gene deletions or disruptions and the host cells are cultivated in the presence of one or more Pmtp inhibitors. In particular aspects of these embodiments, the host cells also express a secreted alpha-1,2-mannosidase.

PMT deletions or disruptions and/or Pmtp inhibitors control O-glycosylation by reducing O-glycosylation occupancy, that is by reducing the total number of O-glycosylation sites on the glycoprotein that are glycosylated. The further addition of an alpha-1,2-mannsodase that is secreted by the cell controls O-glycosylation by reducing the mannose chain length of the O-glycans that are on the glycoprotein. Thus, combining PMT deletions or disruptions and/or Pmtp inhibitors with expression of a secreted alpha-1,2-mannosidase controls O-glycosylation by reducing occupancy and chain length. In particular circumstances, the particular combination of PMT deletions or disruptions, Pmtp inhibitors, and alpha-1,2-mannosidase is determined empirically as particular heterologous glycoproteins (antibodies, for example) may be expressed and transported through the Golgi apparatus with different degrees of efficiency and thus may require a particular combination of PMT deletions or disruptions, Pmtp inhibitors, and alpha-1,2-mannosidase. In another aspect, genes encoding one or more endogenous mannosyltransferase enzymes are deleted. This deletion(s) can be in combination with providing the secreted alpha-1,2-mannosidase and/or PMT inhibitors or can be in lieu of providing the secreted alpha-1,2-mannosidase and/or PMT inhibitors.

Thus, the control of O-glycosylation can be useful for producing particular glycoproteins in the host cells disclosed herein in better total yield or in yield of properly assembled glycoprotein.

Yield of recombinant proteins can in some situations be improved by overexpressing nucleic acid molecules encoding mammalian or human chaperone proteins or replacing the genes encoding one or more endogenous chaperone proteins with nucleic acid molecules encoding one or more mammalian or human chaperone proteins. In addition, the expression of mammalian or human chaperone proteins in the host cell also appears to control O-glycosylation in the cell. Thus, further included are the host cells herein wherein the function of at least one endogenous gene encoding a chaperone protein has been reduced or eliminated, and a vector encoding at least one mammalian or human homolog of the chaperone protein is expressed in the host cell. Also included are host cells in which the endogenous host cell chaperones and the mammalian or human chaperone proteins are expressed. In further aspects, the lower eukaryotic host cell is a yeast or filamentous fungi host cell. Examples of the use of chaperones of host cells in which human chaperone proteins are introduced to improve the yield and reduce or control O-glycosylation of recombinant proteins has been disclosed in U.S. Provisional Application Nos. 61/066,409 filed Feb. 20, 2008 and 61/188,723 filed Aug. 12, 2008; and International Patent Application No. PCT/US2009/033507 filed Feb. 9, 2009, each of which is incorporated by reference in its entirety. Like above, further included are lower eukaryotic host cells wherein, in addition to replacing the genes encoding one or more of the endogenous chaperone proteins with nucleic acid molecules encoding one or more mammalian or human chaperone proteins or overexpressing one or more mammalian or human chaperone proteins as described above, the function or expression of at least one endogenous gene encoding a protein O-mannosyltransferase (PMT) protein is reduced, disrupted, or deleted. In particular embodiments, the function of at least one endogenous PMT gene selected from the group consisting of the PMT1, PMT2, PMT3, and PMT4 genes is reduced, disrupted, or deleted.

Therefore, the methods disclosed herein can use any host cell that has been genetically modified to produce glycoproteins that have no N-glycans compositions wherein the predominant N-glycan is selected from the group consisting of complex N-glycans, hybrid N-glycans, and high mannose N-glycans wherein complex N-glycans are selected from the group consisting of Man₃GlcNAc₂, GlcNAC₍₁₋₄₎Man₃GlcNAc₂, Gal₍₁₋₄₎GlcNAc₍₁₋₄₎Man₃GlcNAc₂, and NANA₍₁₋₄₎Gal₍₁₋₄₎Man₃GlcNAc₂, such as Gal₄GlcNAc₂Man₃GlcNAc₂ (5.9) or NANA₂Gal₂GlcNAc₂Man₃GlcNAc₂ (6.0); hybrid N-glycans are selected from the group consisting of Man₅GlcNAc₂, GlcNAcMan₅GlcNAc₂, GalGlcNAcMan₅GlcNAc₂, Gal₂GlcNAcMan₅GlcNAc₂ and NANAGalGlcNAcMan₅GlcNAc₂; and high Mannose N-glycans are selected from the group consisting of Man₅GlcNAc₂(2.0), Man₆GlcNAc₂, Man₇GlcNAc₂, Man_{g}GlcNAc₂, and Man₉GlcNAc₂.

Yeast cells are transformed with expression constructs described above using a variety of standard techniques including, but not limited to, electroporation, microparticle bombardment, spheroplast generation methods, or whole cell methods such as those involving lithium chloride and polyethylene glycol (Cregg et al., 1985, Mol. Cell. Biol. 5:3376-3385; Liu et al., 1992, J Biol. Chem. 270:10940-10951; Waterham et al., 1996, Mol. Cell. Biol. 16:2527-2536; and Cregg and Russell, 1998, Mol. Biol. 103:27-39).

A preferred expression host in this invention is methylotrophic yeast, examples of which includes suitable strains of Pichia methanolica, Hansenula polymorpha, Pichia pastoris and the like, more preferably Pichia pastoris strain as described earlier. The preferred Pichia pastoris transformants should carry at least one copy of an expression cassette comprising an alcohol-inducible promoter, secretory signal sequence, a novel DNA encoding for an HSV glycoprotein, a transcription termination signal and a selection marker.

Broadly, a fermentation process comprises producing a high-density cell-culture of a glycoengineered Pichia pastoris and expression of heterologous protein under suitable conditions. Various types of fermentation techniques such as batch, fed-batch, and continuous fermentation protocols are well known to those skilled in the art (Brock T. D., Biotechnology: A Textbook of Industrial Microbiology, Sinauer Associates, 2^{nd} Ed., (1989); Demain A. L. and Davies J. E., Manual of Industrial Microbiology and Biotechnology, 2^{nd} Ed., ASM Press, (1999); Hewitt et al., 1999, J. Biotechnol. 75:251). The necessary conditions, equipment and materials required to carryout fermentation by any conventional fermentor are well known (Sherman F., Methods in Enzymology, Guthrie C. et al. (Eds.), Academic Press, N.Y., 194:14, (1991); Hollenberg et al., 1997, Curr. Opin. Biotechnol. 8:554-560). Also standard instrumentation is used to monitor various parameters such as temperature, pH, dissolved oxygen level, amount of nutrients such as carbon source/methanol and nitrogen. All equipment and additives are sterilized according to suitable methods known in prior art.

The typical fermentation protocol of the present invention provides conditions for high-density cell-mass build-up. The protocol has some characteristics of fed batch process of fermentation. The rate of addition of feed supply is related with the growth rate of cells, rate at which carbon and nitrogen are assimilated and also with CIH/UN content of the cells.

A typical production process comprises of cells cultured in liquid medium at about 25° C. to 35° C. under aerated condition. Also it is known that in the case of Pichia pastoris the design of fermentor is an important factor during the process optimization (Ellis et al., 1985, Mol. Cell. Biol. 9:1316-1323; Villatte et al., 2001, Appl. Microbiol. Biotechnol. 55:463-5; Morganti et al., 1996, Biotechnol. Appl. Biochem. 23:67; Stratton J. et al., Pichia Protocols, Higgins D. R., and Cregg J. M. (Eds.), Humana Press, Totowa, N.J.) as also described in the prior art (International Patent Application Publication No. WO 90/03431; Phillips A., et al, Methods in Enzymol., Academic Press, N.Y., 119:35-38 (1986)). High aeration requirement may be provided by type, design and length of sparger, and by adjusting the agitation speed, air and oxygen supply based on dissolved oxygen concentration of fermentation broth and cell density. Exhaustion of glycerol leads to arrest of the logarithmic growth phase. At this point glycerol feed is initiated, and the feed rate is adjusted depending upon cell mass build up and utilization.

Culture medium includes a suitable source for carbon such as glucose, glycerol sucrose etc., assimilable nitrogen such as nitrates, NH₄ as ammonical liquor, yeast nitrogen base etc., along with vitamins such as vitamin B₁₂, essential amino acids such as histidine, biotin, methionine etc., mineral supplements and trace metals such as manganese, mercury, iron and molybdenum salts, phosphates, sulfates etc. During fermentation, there can be single or multiple ingredients acting as a source for carbon to the growing cell culture. Suitable carbon sources include compounds, such as glycerol, glucose, fructose and the like, preferably glycerol. Alternatively, carbon source can include lower alcohols such as methanol, ethanol, propanol, isopropanol, butanol, isobutanol, and the like, preferably methanol. The examples of which include aqueous solution or syrups made using glucose or fructose, preferably aqueous solution. Glycerol may be used as the sole carbon source or 40% of glycerol can be mixed with aqueous solution containing other nutrients required by the yeast. Alcohol content of the media can range from about 0.1% to about 3%. For example, medium can contain alcohol about 0.5%, 1%, 2%, or 3%.

In one embodiment, the fermentation is conducted in a manner that the carbon source is a growth-limiting factor and thereby providing good conversion of the carbon source into higher cell mass buildup.

The assimilable nitrogen can be supplied using any nitrogen containing compounds capable of releasing nitrogen in a form that can be utilized by the yeast. Examples of nitrogen source includes organic or inorganic substances such as ammonium salts, nitrates, corn steep liquor, peptone, casein, meat extracts, bean-cakes, potato extracts, protein hydrolysates, yeast extract, urea, ammonium hydroxide and the like, more preferably aqueous ammonia solution.

The media can also contain high level of inorganic salts, such as magnesium, maganese, copper, sodium, molybdenum, zinc, iron, potassium, calcium sulfate, phosphoric acid, orthophosphoric acid, sulfuric acid, boric acid and the like; vitamins such as biotin, thiamine and the like; protease inhibitors; amino acids such as histidine and the like; along with other trace nutrients and metals. Nevertheless, the medium can be supplemented with acid hydrolyzed casein (e.g., casamino acids or amicase) if desired to provide an enriched medium. In addition, media can also contain yeast's processing additives, growth-promoting factors, etc.

The pH range in the aqueous microbial ferment may be in the range of 4 to 7, preferably around 4.5 to about 6.5. The preferred temperature during the fermentation is around 25° C.-30° C.

The Pichia yeast requires aerobic conditions for growth, hence dissolved oxygen is required at all times during the fermentation. This may include supply of molecular oxygen in the form of air, oxygen enriched air or pure molecular oxygen itself so as to maintain the ferment with sufficient dissolved oxygen necessary to assist growth of cell. The overall aeration rates may vary from about 0.3 to 1.0 VVM (volume air per volume of ferment per minute). The level of dissolved oxygen in the culture medium may vary from a minimum of about 1% to about 100% saturation, more preferably about 30% to about 80% saturation, and most preferably about 20-60% saturation. During growth stage, the dissolved oxygen concentration may vary during the initial stages depending upon agitation (stirrer speed) in the fermentor.

To achieve high cell density in the fermentation, a fed batch fermentation protocol may be suitably modified. This may involve addition of suitable nutrients and carbon source. Alternatively, the batch can be modified by supplying booster feed of suitable nutrients from external source.

After the suitable growth phase, protein production may be induced using a suitable alcohol, selected from the group consisting of methanol, ethanol, propanol, isopropanol, butanol, and isobutanol, preferably methanol. Suitable time for starting the production phase can be between 48 to 110 hours of cell growth and the biomass achieved is approximately 100 to 200 g/L of wet weight. Induction of protein production is started by addition of methanol. Methanol is added in the concentration range of about 0.6 to 3.0% v/v preferably 0.8 to 2.8% v/v, which is monitored by gas chromatography. The production medium feed may deliver methanol in neat form or the alcohol may be diluted initially with suitable amounts of water or trace metal solution. Optionally along with a slow feed of methanol, glycerol may be provided for a short time to retain metabolic activity of the cells.

It is not necessary to continuously add methanol for the entire production phase of fermentation process in the present invention. According to a preferable embodiment of the present invention, a medium may contain 1 to 2% v/v methanol at the start of production phase of fermentation process. Production phase is monitored every 4-6 hours by sampling and examining various parameters which includes pH, OD, methanol concentration and increase in the concentration of the desired protein. Addition of methanol is controlled accordingly. Continuous or periodic addition of methanol is then started when the methanol concentration decreases to about 0.5% v/v or less, and for example, 0.2 to 0.5% v/v. In case the methanol concentration in the medium continues to decrease, eventually falling to 0% to 0.1% v/v the continuous addition may be started, and continued until a methanol concentration of 2.0 to 3.0% v/v is attained.

During the time of methanol addition in this manner, the promoter is induced by methanol causing expression of the target gene, which encodes for an HSV glycoprotein. In addition to this, the added methanol may be partly used for the growth of the microorganisms. The preferred embodiment of this invention involves addition of medium or extra nitrogen source prior to induction, which may lead to growth of the microorganisms simultaneous to the protein production, over at least a certain period of time.

Purification of Heterologous Protein Obtained from Transformed Pichia

Methods for recovering recombinant proteins obtained from transformed yeast cells are well known in the art. See, e.g., Romanos et al., DNA Cloning 2: Expression Systems, IRL Press, 2nd Ed., pages 123-167 (1995); Trona et al., Developments in Industrial Microbiology, Elsevier, Amsterdam, 53-64 (1987); Nagabhushan T. L. and Trona P. P., Ullmann's Encyclopedia of Industrial Chemistry, A14, VCH, Weinheim, Federal Republic of Germany 372-374 (1989). Standard techniques, such as affinity chromatography, size exclusion chromatography, ion exchange chromatography, HPLC and the like can be used to purify the protein of interest.

Typically, secreted proteins can have purity anywhere in between 20-50%. The expressed polypeptide can be further purified to 90% purity; or even greater than 95% purity with respect to contaminating macromolecules, particularly other proteins, nucleic acids, and other infectious and pyrogenic agents. Polypeptides expressed by methylotrophic yeast may also be purified to a pharmaceutically pure state, which is greater than 99.0% pure.

In cases where proteins are secreted into culture media, advantage is of relatively lower contaminating substances, and the supernatant can be collected by known methods to isolate proteins. See, e.g., Berg, Acta Path. Microbiol. Immunol. Scand., Section C, Suppl. 279, 1-136 (1982); Berg K, and Heron I., Methods in Enzymology, Academic Press, N.Y., 78, 487-499 (1981); U.S. Pat. No. 4,289,690. This culture supernatant containing expressed protein can be purified by any one or more than one method in combination. The standard methods of protein purification are based on differences in the physicochemical characteristics of the proteins. The various methods are enlisted below:
i) solubility difference, examples of which include methods such as salting out, precipitation with solvents,
ii) differences in molecular size or weight, examples include dialysis, ultrafiltration, gel filtration and SDS-polyacrylamide gel electrophoresis,
iii) difference in the electric charge, examples include ion-exchange chromatography,
iv) methods utilizing specific affinity, examples include affinity chromatography,
v) methods utilizing a difference in the hydrophobic property, examples include reversed-phase high-performance liquid chromatography or Hydrophobic Interaction Chromatography and
vi) methods utilizing a difference in the isoelectric point examples include isoelectric electrophoresis, etc.

In order to obtain the native protein in its correctly folded state, it is preferable to use processes which avoids denaturation and precipitation steps.

Suitable chromatographic media include derivatized dextrans, agarose, cellulose, polyacrylamide, specialty silicas and the like, including PEI, DEAE, QAE, and Q derivatives. Examples of chromatographic media include those media derivatized with phenyl, butyl, or octyl groups, such as Phenyl-Sepharose FF (Pharmacia), Toyopearl butyl 650 (Toso Haas, Montgomeryville, Pa.), Octyl-Sepharose (Pharmacia) and the like, or polyacrylic resins, such as Amberchrom CG 71 (Toso Haas) and the like. Suitable solid supports include glass beads, silica-based resins, cellulosic resins, agarose beads, cross-linked agarose beads, polystyrene beads, cross-linked polyacrylamide resins and the like that are insoluble under the conditions in which they are to be used. Optionally, one can modify these supports with reactive groups such that proteins can link with amino, carboxyl, sulfhydryl, hydroxyl and/or carbohydrate moieties from the protein. Also, it is possible to engineer a tag onto the amino- or carboxyl-terminus of the recombinant protein to allow purification by affinity chromatography.

In cases where the protein thus obtained is in a free form, the free protein can be converted into a salt thereof by known methods or method analogous thereto. In case, where the protein thus obtained is in a salt form vice versa, the protein salt can be converted into a free form or into any other salt thereof by known methods or method analogous thereto. The suitable buffer solution may contain a protein-denaturing agent such as urea or guanidine hydrochloride or a surfactant such as Triton X-100™.

### Pharmaceutical/Vaccine Compositions

The present invention further provides compositions, including pharmaceutical, immunogenic and vaccine compositions, comprising a) a recombinant glycoprotein of the invention, wherein said glycoprotein is gD protein or immunogenic fragment thereof, b) a recombinant HSV gC protein or immunogenic fragment thereof, and c) an adjuvant; immunogenic compositions comprising a) a recombinant HSV gD protein or immunogenic fragment thereof, b) a recombinant glycoprotein of the invention, wherein said glycoprotein is gC protein or immunogenic fragment thereof, and c) an adjuvant; and immunogenic compositions comprising a) a recombinant glycoprotein of the invention, wherein said glycoprotein is gD protein or immunogenic fragment thereof, b) a recombinant glycoprotein of the invention, wherein said glycoprotein is gC protein or immunogenic fragment thereof, and c) an adjuvant. In certain embodiments, the immunogenic compositions comprise gD protein and gC protein which are HSV-2 proteins. In other embodiments, the immunogenic compositions comprise gD protein and gC protein which are HSV-1 proteins. The immunogenic compositions may further comprise one or more recombinant HSV-2 proteins or HSV-1 proteins selected from a gB protein and a gE protein.

Compositions of the invention, including pharmaceutical, immunogenic and vaccine compositions, preferably comprise gD, or a fragment thereof, and gC, or a fragment thereof, and an adjuvant. At least one of gD or gC is a recombinant glycoprotein of the invention having the specified mole percent of a particular glycoprotein. In certain embodiments, both gD and gC are recombinant glycoprotein of the invention having the specified mole percent of a particular glycoprotein. In certain embodiments, a composition comprises (i) a gC-2 having an amino acid sequence of SEQ ID NO: 2, and/or (ii) gD 310 or gD 339 is used. In other embodiments, a composition comprises (i) a gC having 95% homology to SEQ ID NO: 2, and/or (ii) a gD having 95% homology to gD 310 or gD 339. In other embodiments, a composition comprises (i) a gC having up to 20 amino acid changes (in any combination) compared to SEQ ID NO: 2, and/or (ii) a gD having up to 20 amino acid changes (in any combination) compared to gD 310 or gD 339.

gC binds complement components C3, C3b, iC3b, and C3c and is one of three immune evasion molecules that is expressed on the cells surface and incorporated into the virion making it an attractive target for vaccines. Mice immunized with a baculovirus-expressed ectodomain of HSV-1 gC (gCt-1) were protected from HSV-1 challenge. See Talsinger et al., 1995, J Virol 69:4471-4483. No neutralizing antibodies were detected; thus, it was concluded that protection was mediated by antibodies that block C3b binding to gC. gC-2 has modest measurable neutralizing antibody activity and also elicits antibodies that block C3b binding to gC. Thus, for gC-2, protection was mediated by both mechanisms for HSV-2.

Glycoprotein D (gD) is found inserted in the membrane and displays an ectodomain on the external surface of the membrane. This region has been shown to specifically bind to three distinct cellular receptors on the host cell thereby aiding in viral entry. Roles for gD have also been identified in blocking apoptosis and in virion egress. gD binds to several cell surface proteins including nectin-1, nectin-2, herpes entry mediator (HVEM) and 3-O-sulfonated derivatives of heparan sulfate. Binding to any one of these receptors is thought to initiate conformational changes in gD (Krummenacher et al., 2005, EMBO J. 24:4144-4153) that lead to changes in conformation of viral glycoproteins gB and gHgL (Gianni et al., 2009, J. Biol. Chem. 284: 17370-17382). It is the latter 3 proteins that mediate fusion of viral and cellular membranes resulting in deposition of the viral genome into the cytoplasm of the cell. Antibodies elicited by the gD ectodomain have neutralization potential and have been shown to have some efficacy against HSV-2 disease. See Stanberry et al., 2002, New Engl J Med 347:1652-1661.

In certain embodiments, the present invention provides pharmaceutical compositions comprising one or more recombinant HSV proteins selected from the group consisting of gC, gD, gB and gE, at least one of which is produced in Pichia pastoris.

In another embodiment, a composition of the invention comprises recombinant HSV-1 proteins. In another embodiment, the vaccine comprises recombinant HSV-2 proteins. In another embodiment, the vaccine comprises both HSV-1 and HSV-2 proteins. Preferably, the HSV ectodomains are HSV-2.

In one embodiment, a recombinant HSV-1-protein-containing vaccine of the present invention further comprises a recombinant HSV-2 protein or fragment thereof. The recombinant HSV-2 protein is a gD2 protein or fragment thereof, a gC2 protein or fragment thereof, a gE2 protein or fragment thereof, a gB2 protein of fragment thereof, or any combination thereof.

In another embodiment, a recombinant HSV-2-protein-containing vaccine of the present invention further comprises a recombinant HSV-1 protein. The recombinant HSV-1 protein is a gD1 protein or fragment thereof, a gC 1 protein or fragment thereof, a gE1 protein or fragment thereof, a gB2 protein or fragment thereof, or any combination thereof.

In another embodiment, the present invention provides a kit comprising a vaccine utilized in performing a method of the present invention. In another embodiment, the present invention provides a kit comprising a vaccine of the present invention.

### Adjuvants

In certain embodiments, the compositions of the present invention comprise an adjuvant. “Adjuvant” refers to compounds that, when administered to an individual or tested in vitro, increase the immune response to an antigen in the individual or test system to which the antigen is administered. An immune adjuvant may enhance an immune response to an antigen that is weakly immunogenic when administered alone, i.e., inducing no or weak antibody titers or cell-mediated immune response, increase antibody titers to the antigen, and/or lowers the dose of the antigen effective to achieve an immune response in the individual.

In certain embodiments, the adjuvant is an aluminum salt adjuvant. The aluminum salt adjuvant may be an alum-precipitated vaccine or an alum-adsorbed vaccine. Aluminum-salt adjuvants are well known in the art and are described, for example, in Harlow, E. and D. Lane (1988; Antibodies: A Laboratory Manual Cold Spring Harbor Laboratory) and Nicklas, W. (1992; Aluminum salts. Research in Immunology 143:489-493). The aluminum salt includes, but is not limited to, hydrated alumina, alumina hydrate, alumina trihydrate (ATH), aluminum hydrate, aluminum trihydrate, alhydrogel, Superfos, Amphogel, aluminum (III) hydroxide, aluminum hydroxyphosphate sulfate (Merck Aluminum Adjuvant (MAA); see Caulfield et al., 2007, Human Vaccines 3:139-145), amorphous alumina, trihydrated alumina, trihydroxyaluminum, or any other aluminum salt known in the art.

In certain embodiments, a commercially available Al(OH)₃ (e.g. Alhydrogel or Superfos of Denmark/Accurate Chemical and Scientific Co., Westbury, N.Y.) is used to adsorb proteins in a ratio of 50-200 g protein/mg aluminum hydroxide. Adsorption of protein is dependent, in another embodiment, on the pI (Isoelectric pH) of the protein and the pH of the medium. A protein with a lower pI adsorbs to the positively charged aluminum ion more strongly than a protein with a higher pI. Aluminum salts may establish a depot of Ag that is released slowly over a period of 2-3 weeks, nonspecific activation of macrophages and complement activation.

In a particular embodiment, the vaccine is formulated with aluminum hydroxyphosphate sulfate, e.g., Merck aluminum adjuvant, plus a saponin-based adjuvant, e.g., Iscomatrix®.

In certain embodiments, the adjuvant is a CpG-containing nucleotide sequence, for example, a CpG-containing oligonucleotide, in particular, a CpG-containing oligodeoxynucleotide (CpG ODN). In another embodiment, the adjuvant is ODN 1826, which may be acquired from Coley Pharmaceutical Group.

“CpG-containing nucleotide,” “CpG-containing oligonucleotide,” “CpG oligonucleotide,” and similar terms refer to a nucleotide molecule of 8-50 nucleotides in length that contains an unmethylated CpG moiety. In another embodiment, any other art-accepted definition of the terms is intended.

In another embodiment, a CpG-containing oligonucleotide is a modified oligonucleotide. “Modified oligonucleotide” refers, in another embodiment, to an oligonucleotide in which at least two of its nucleotides are covalently linked via a synthetic internucleoside linkage (i.e., a linkage other than a phosphodiester linkage between the 5′ end of one nucleotide and the 3¹ end of another nucleotide), for example, a chemical group not normally associated with nucleic acids has been covalently attached to the oligonucleotide. Representative synthetic internucleoside linkage include, but are not limited to, a phosphorothioate linkage, an alkylphosphonate linkage, a phosphorodithioate linkage, a phosphate ester linkage, an alkylphosphonothioate linkage, a phosphoramidate linkage, a carbamate linkage, a carbonate linkage, a phosphate trimester linkage, an acetamidate linkage, a carboxymethyl ester linkage, or a peptide linkage.

In another embodiment, the term “modified oligonucleotide” refers to oligonucleotides with a covalently modified base and/or sugar. In another embodiment, modified oligonucleotides include oligonucleotides having backbone sugars covalently attached to low molecular weight organic groups other than a hydroxyl group at the 3′ position and other than a phosphate group at the 5′ position. In another embodiment, modified oligonucleotides include a 2′-O-alkylated ribose group, or arabinose. In another embodiment, modified oligonucleotides include murine TLR9 polypeptides, together with pharmaceutically acceptable carriers.

In another embodiment, the CpG-containing oligonucleotide is double-stranded. In another embodiment, the CpG-containing oligonucleotide is single-stranded. In another embodiment, “nucleic acid” and “oligonucleotide” refer to multiple nucleotides (i.e., molecules comprising a sugar (e.g. ribose or deoxyribose) linked to a phosphate group and to an exchangeable organic base, which is either a substituted pyrimidine (e.g. cytosine (C), thymine (T) or uracil (U)) or a substituted purine (e.g. adenine (A) or guanine (G)) or a modified base. In another embodiment, the terms refer to oligoribonucleotides as well as oligodeoxyribonucleotides. In another embodiment, the terms include polynucleosides (i.e., a polynucleotide minus the phosphate) and any other organic base-containing polymer. In another embodiment, the terms encompass nucleic acids or oligonucleotides with a covalently modified base and/or sugar, as described herein.

In another embodiment, a CpG-containing oligonucleotide of methods and compositions of the present invention comprises a substituted purine and pyrimidine. In another embodiment, the oligonucleotide comprises standard purines and pyrimidines such as cytosine as well as base analogs such as C-5 propyne-substituted bases (Wagner et al., 1996, Nat Biotechnol 14:840-844. In another embodiment, purines and pyrimidines include but are not limited to adenine, cytosine, guanine, thymine, 5-methylcytosine, 2-aminopurine, 2-amino-6-chloropurine, 2,6-diaminopurine, hypoxanthine, and other naturally and non-naturally occurring nucleobases, substituted and unsubstituted aromatic moieties. In another embodiment, CpG-containing oligonucleotide is a linked polymer of bases or nucleotides. In another embodiment, “linked” refers to 2 entities bound to one another by any physicochemical means.

Methods for use of CpG oligonucleotides are well known in the art and are described, for example, in Sur et al., 1999, J. Immunol. 162:6284-93; Verthelyi, 2006, Methods Mol. Med. 127:139-58; and Yasuda et al., 2006, Crit. Rev Ther Drug Carrier Syst. 23:89-110.

Methods for production of nucleic acids having modified backbones are well known in the art, and are described, for example in U.S. Pat. Nos. 5,723,335 and 5,663,153 and International Patent Publication No. WO95/26204.

Other suitable adjuvants include, but are not limited to, a Montanide ISA adjuvant, a trimer of complement component C3d (which is optionally covalently linked to the protein immunogen), MF59, a granulocyte/macrophage colony-stimulating factor (GM-CSF) protein or a mixture comprising a GM-CSF protein, a nucleotide molecule encoding GM-CSF or a mixture comprising a nucleotide molecule encoding GM-CSF, a saponin-based adjuvant such as QS21 or Iscomatrix®, or a mixture comprising a saponin-based adjuvant, monophosphoryl lipid A (MPL) or a mixture comprising MPL, SBAS2 or a mixture comprising SBAS2, an unmethylated CpG-containing oligonucleotide or a mixture comprising an unmethylated CpG-containing oligonucleotide, an immune-stimulating cytokine or a mixture comprising an immune-stimulating cytokine, a nucleotide molecule encoding an immune-stimulating cytokine or a mixture comprising a nucleotide molecule encoding an immune-stimulating cytokine, a mixture comprising a quill glycoside, a mixture comprising a bacterial mitogen, a mixture comprising a bacterial toxin, ISCOM (immunostimulating complexes formed by the combination of cholesterol, saponin, phospholipid, and amphipathic proteins) and Iscom Matrix (having essentially the same structure as an ISCOM but without the protein), or a mixture comprising any other adjuvant known in the art. In another embodiment, the adjuvant is a mixture of 2, 3, or more of the above adjuvants.

In another embodiment, the adjuvant is a carrier polypeptide. “Carrier polypeptide” refers to a protein or immunogenic fragment thereof that can be conjugated or joined with an HSV protein of the present invention to enhance immunogenicity of the polypeptide. Examples of carrier proteins include, but are by no means limited to, keyhole limpet Hemocyanin (KLH), albumin, cholera toxin, heat labile enterotoxin (LT), and the like. In another embodiment, the 2 components are prepared as a chimeric construct for expression as a fusion polypeptide. In another embodiment, chemical cross-linking is used to link an HSV protein with a carrier polypeptide.

### Administration

In one embodiment, the present invention provides a method of vaccinating a subject against an HSV infection, the method comprising the step of administering to said subject a vaccine of the present invention. This method may reduce the incidence of an HSV disease and/or HSV infection, prevent a recurrence of an HSV infection, diminish the severity of a recurrence of an HSV infection and/or reduce the frequency of a recurrence of an HSV infection. In certain embodiments, the subject is an HIV-infected subject.

In another embodiment, the present invention provides a method of treating an HSV infection in a subject, the method comprising the step of administering to said subject a composition of the present invention. Such treating encompasses inhibiting, suppressing or impeding an HSV infection in a subject, and includes inhibiting spread of HSV (e.g., spread from DRG or TG to skin, cell-to-cell spread, spread to neural tissue, anterograde or retrograde spread), which may be a primary HSV infection, and inhibiting or preventing a flare or recurrence following a primary HSV infection.

In other embodiments, administration of a composition of the invention inhibits an HSV labialis following a primary HSV infection, treats an HSV encephalitis, reduces an incidence of an HSV encephalitis, treats or reduces an HSV neonatal infection, reduces an incidence of an HSV-mediated herpetic ocular disease, treats an HSV-1 corneal infection or herpes keratitis, reduces an incidence of an HSV-1 corneal infection or herpes keratitis, treats, suppresses or inhibits an HSV genital infection, treats, suppresses or inhibits any manifestation of recurrent HSV infection, reduces an incidence of an HSV-mediated genital ulcer disease, impedes an establishment of a latent HSV, protects against formation of a zosteriform lesion or an analogous outbreak, or inhibits the formation of an HSV zosteriform lesion or an analogous outbreak. The herpes-mediated encephalitis treated or prevented by a method of the present invention is a focal herpes encephalitis, a neonatal herpes encephalitis, or any other type of herpes-mediated encephalitis known in the art.

In another embodiment, the present invention provides a method of treating or reducing an incidence of a disease, disorder, or symptom associated with or secondary to a HSV-mediated encephalitis in a subject, the method comprising the step of administering to said subject a vaccine of the present invention.

In another embodiment, the present invention provides a method of treating, reducing the pathogenesis of, ameliorating the symptoms of, ameliorating the secondary symptoms of, reducing the incidence of, prolonging the latency to a relapse of an HSV infection in a subject, comprising the step of administering to the subject a vaccine of the present invention.

It is to be understood that the methods of the present invention may be used to treat, inhibit, suppress, etc an HSV infection or primary or secondary symptoms related to such an infection following exposure of the subject to HSV. In certain embodiments, the subject has been infected with HSV before vaccination or is at risk for HSV infection. In another embodiment, whether or not the subject has been infected with HSV at the time of vaccination, vaccination by a method of the present invention is efficacious in treating, inhibiting, suppressing, etc an HSV infection or primary or secondary symptoms related to such an infection.

In one embodiment, symptoms are primary, while in another embodiment, symptoms are secondary. In one embodiment, “primary” refers to a symptom that is a direct result of the subject viral infection, while in one embodiment, “secondary” refers to a symptom that is derived from or consequent to a primary cause. In one embodiment, the compositions and strains for use in the present invention treat primary or secondary symptoms or secondary complications related to HSV infection.

In another embodiment, “symptoms” may be any manifestation of a HSV infection, comprising blisters, ulcerations, or lesions on the urethra, cervix, upper thigh, and/or anus in women and on the penis, urethra, scrotum, upper thigh, and anus in men, inflammation, swelling, fever, flu-like symptoms, sore mouth, sore throat, pharyngitis, pain, blisters on tongue, mouth or lips, ulcers, cold sores, neck pain, enlarged lymph nodes, reddening, bleeding, itching, dysuria, headache, muscle pain, etc., or a combination thereof.

In another embodiment, the disease, disorder, or symptom is fever, headache, stiff neck, seizures, partial paralysis, stupor, coma, or any other disease, disorder, or symptom known in the art that is associated with or secondary to a herpes-mediated encephalitis.

Methods of determining the presence and severity of herpes-mediated encephalitis are well known in the art, and are described, for example, in Bonkowsky et al. 2006, Pediatrics 117:1045-8 and Khan et al. 2006, BMC Fam Pract. 7:22.

In another embodiment, the present invention provides a method of treating or reducing an incidence of a disease, disorder, or symptom associated with an HSV infection in a subject, the method comprising the step of administering to said subject a vaccine of the present invention.

The disease, disorder, or symptom secondary to an HSV infection is oral lesions, genital lesions, oral ulcers, genital ulcers, fever, headache, muscle ache, swollen glands in the groin area, painful urination, vaginal discharge, blistering, flu-like malaise, keratitis, herpetic whitlow, Bell's palsy, herpetic erythema multiforme, a lower back symptom (e.g. numbness, tingling of the buttocks or the area around the anus, urinary retention, constipation, and impotence), a localized eczema herpeticum, a disseminated eczema herpeticum, a herpes gladiatorum, a herpetic sycosis, an esophageal symptom (e.g. difficulty swallowing or burning, squeezing throat pain while swallowing, weight loss, pain in or behind the upper chest while swallowing), or any other disease, disorder, or symptom that is known in the art.

In certain embodiments, the HSV infection that is treated or ameliorated by methods and compositions of the present invention is a genital HSV infection, an oral HSV infection, an ocular HSV infection, or a dermatologic HSV infection.

Vaccine administration may also be used for reducing an incidence of a disseminated HSV infection, reducing an incidence of a neonatal HSV infection in an offspring, reducing a transmission of an HSV infection from a subject to an offspring thereof, and reducing a severity of a neonatal HSV infection in an offspring.

In another embodiment, the offspring is an infant. In another embodiment, the transmission that is reduced or inhibited is transmission during birth. In another embodiment, transmission during breastfeeding is reduced or inhibited. In another embodiment, the transmission that is reduced or inhibited is any other type of parent-to-offspring transmission known in the art.

In certain embodiments of the invention, the subject is infected with HIV. A subject may be infected with or susceptible to infection with HSV or at least one other pathogen. A subject may be immunocompromised, infected by HSV, at risk for infection by HSV, immunocompromised, and/or elderly. The compositions or vaccines of the present invention and their related uses may suppress, inhibit, prevent or treat an HSV infection in an HIV-infected subject. In one embodiment, the HIV-infected subject may have CD4 T-cell counts lower than 200 μl, in another embodiment, the HIV-infected subject may have CD4 T-cell counts between 200-500 μl, or in another embodiment, the HIV-infected subject may have CD4 T-cell counts greater than 500/μl. In one embodiment, HIV-infected subjects have high hemolytic serum complement (CH50) levels, while in another embodiment, HIV-infected subjects have low CH50 levels.

Methods of determining the extent of HSV replication and HSV infection are well known in the art, and are described, for example, in Lambiase, 2007, Graefes Arch Clin Exp Ophthalmol 246:121-7, Ramaswamy et al., 2007, Expert Rev Anti Infect Ther. 5:231-43, and Jiang et al., 2007, J. Virol. 81:3495-502.

Methods for determining the presence and extent of herpetic ocular disease, corneal infection, herpes keratitis are well known in the art, and are described, for example, in Labetoulle et al., 2000, Invest Ophthalmol V is Sci. 41:2600-6, and Majumdar et al., 2005, J Ocul Pharmacol Ther. 21:463-74.

In another embodiment, administration of the vaccine to a human subject elicits an anti-HSV-1 gC antibody that blocks an immune evasion function of an HSV protein corresponding to the recombinant HSV-1 gC protein. In another embodiment, the gC fragment includes an immune evasion domain thereof. In another embodiment, immunization with gC and gD, or fragments thereof, in combination limits the ability of HSV-1 to evade a host immune response during a subsequent challenge. In another embodiment, immunization with gC or a fragment thereof limits the ability of HSV-1 to evade a host anti-gD immune response during a subsequent challenge. In another embodiment, the host immune response referred to comprises anti-gD antibodies induced by the vaccine. In another embodiment, the immune-potentiating effect of gC and gD, or fragments thereof, together is greater than the effect of either alone. In another embodiment, the immune-potentiating effects of gC and gD exhibit synergy.

In another embodiment, administration of the vaccine to a human subject elicits an anti-HSV-2 gC antibody that blocks an immune evasion function of an HSV protein corresponding to the recombinant HSV-2 gC protein. In another embodiment, the gC fragment includes an immune evasion domain thereof. In another embodiment, immunization with gC and gD, or fragments thereof, in combination limits the ability of HSV-2 to evade a host immune response during a subsequent challenge. In another embodiment, immunization with gC or a fragment thereof, limits the ability of HSV-2 to evade a host anti-gD immune response during a subsequent challenge. In another embodiment, the host immune response referred to comprises anti-gD antibodies induced by the vaccine.

In another embodiment, a vaccine of the present invention elicits antibodies that inhibit binding of gD to a cellular receptor. In another embodiment, the receptor is herpesvirus entry mediator A (HveA/HVEM). In another embodiment, the receptor is nectin-1 (HveC). In another embodiment, the receptor is nectin-2 (HveB). In another embodiment, the receptor is a modified form of heparan sulfate. In another embodiment, the receptor is a heparan sulfate proteoglycan. In another embodiment, the receptor is any other gD receptor known in the art.

In another embodiment, a vaccine regimen of the present invention further comprises the step of administering to the subject a booster vaccination, wherein the booster vaccination comprises one or more of recombinant HSV gB, gC, gD, or gE protein, or an immunogenic fragment thereof, used in the priming vaccination, but not the other recombinant proteins present in the priming vaccinations. The HSV proteins may be any combination of HSV-1 or HSV-2 proteins.

In another embodiment, the booster vaccination follows a single priming vaccination. “Priming vaccination” refers to a vaccination that comprises two or more recombinant HSV proteins selected from a gD protein, a gC protein, gB protein and a gE protein. The term may refer to a vaccine initially administered, or alternatively, two, three or four priming vaccinations are administered before the booster vaccination. One, two or three booster vaccination may be administered after the priming vaccinations.

The HSV protein may be administered in a single syringe at the same site, in separate syringes at separate sites, or are administered simultaneously at a single site and followed by a booster dose.

### Dosage

The dose of recombinant HSV glycoprotein, or fragment thereof, utilized in a vaccination or in a booster vaccination is, for example, for a human subject, 10, 25, 50, 100, 150, 200, 250, 300, 400, 500, or 750 ng/inoculation or 1, 1.5, 2, 3, 4, 5, 6, 7, 7.5, 8, 9, 10, 11, 12, 13, 14, 15, 16, 18, 20, 22, 25, 30, 40, 50, 60, 70, 80, 90, 100, 110, 120, 130, 140, 150, 160, 180, 200, 250, 300, 400, or 500 μg/inoculation.

In another embodiment, the dosage is 0.02, 0.03, 0.04, 0.05, 0.06, 0.07, 0.08, 0.09, 0.1, 0.11, 0.12, 0.13, 0.15, 0.2, 0.5, 1.25, 2.5, 5, 10, 12.5, 25, 50, 100, 200, 250, 300, or 500 μg/kg body mass (per inoculation). In another embodiment, the dosage is less than 0.02 μg/kg. In another embodiment, the dosage is 0.01-0.02, 0.02-0.03, 0.02-0.04, 0.03-0.06, 0.04-0.08, 0.05-0.1, 0.05-0.15, 0.1-0.2, 0.2-0.3, 0.3-0.4, 0.4-0.6, 0.5-0.8, or 0.8-1 μg/kg body mass (per injection).

In another embodiment, the dosage is 10-100, 50-250, 10-250, 100-500, or 200-300 ng/inoculation. In another embodiment, the dosage is 0.5-2, 0.5-10, 1-2, 2-3, 2-4, 2.5-7.5, 3-6, 4-8, 5-10, 5-15, 10-20, 20-30, 30-40, 40-60, 2-50, 3-50, 5-50, 8-50, 10-50, 20-50, 20-100, 30-100, 50-100, 80-100, 2-200, 30-200, 50-200, 80-200, 100-200, 2-300, 30-300, 50-300, 80-300, 100-300, 200-300, 2-500, 30-500, 50-500, 80-500, 100-500, 200-500, or 300-500 μg/inoculation. In another embodiment, the dosage is 1-2, 2-3, 2-4, 2-50, 3-6, 3-50, 4-8, 5-10, 5-15, 5-50, 8-50, 10-20, 10-50, 20-30, 20-50, 30-40, 40-60 μg/protein/inoculation.

In one embodiment, the dose of the alum salt is 10, 15, 20, 25, 30, 50, 70, 100, 150, 200, 300, 500, or 700 μg, or 1, 1.2, 1.5, 2, 3, 5 mg or more. In another embodiment, the dose of the alum salt is 10-100, 2-100, 30-100, 50-100, 100-200, 150-300, 200-400, 300-600, 500-1000, or 700-1500 μg or 1-2, 1.5-2, 2-3, 3-5, or 5-8 mg. In yet another embodiment, the dose of alum salt described above is per μg of recombinant protein.

The dose of the CpG oligonucleotide or saponin-based adjuvant (e.g., Iscomatrix®) is 10, 20, 30, 50, 70, 100, 150, 200, 300, 500, or 700 μg. In another embodiment, the dose is 1, 12, 1.5, 2, 3, 5 mg or more.

In another embodiment, the dose of the CpG oligonucleotide or saponin-based adjuvant (e.g., Iscomatrix®) is 10-100, 10-30, 20-100, 30-100, 50-100, 100-200, 100-250, 50-250, 150-300, 200-400, 250-500, 300-600, 500-1000, or 700-1500 μg. In another embodiment, the dose is 0.25-2, 0.5-2, 1-2, 1.5-2, 2-3, 3-5, or 5-8 mg.

The booster vaccination may comprise an adjuvant. The adjuvant may comprise a CpG oligonucleotide, ISCOM, an aluminum salt, any other adjuvant disclosed above, or a combination thereof. Dosages of the adjuvant in a booster vaccination may be any dosage disclosed above.

According to any of the methods of the present invention and in one embodiment, the subject is human. In another embodiment, the subject is a rodent, such as a mouse, or a guinea pig. In another embodiment, the subject is canine, feline, bovine, ovine, porcine, a rat or lagomorph. In another embodiment, the subject is mammalian. In another embodiment, the subject is any organism susceptible to infection by HSV.

In another embodiment, the dosage of the recombinant HSV protein for human vaccination is determined by extrapolation from animal studies to human data. In another embodiment, the dosage of recombinant HSV protein is determined by using a ratio of protein efficacious in human vs. guinea pig studies. In another embodiment, the dosage of recombinant HSV protein for human vaccination is determined empirically.

In another embodiment, utilization of an adjuvant of the present invention enables a lower effective dosage of the recombinant HSV protein. In another embodiment, a lower effective dosage of a recombinant HSV protein is enabled by combination with another recombinant HSV protein, e.g., gD with gC immunogen. In another embodiment, a still lower effective dosage of a recombinant HSV protein and another recombinant HSV protein, e.g., gD in combination with gE, and an adjuvant of the present invention.

“Effective dose” of a glycoprotein refers, in another embodiment, the dose required to elicit antibodies that significantly block an immune evasion function of an HSV virus during a subsequent challenge. In another embodiment, the term refers to the dose required to elicit antibodies that effectively block an immune evasion function of an HSV virus during a subsequent challenge. In another embodiment, the term refers to the dose required to elicit antibodies that significantly reduce infectivity of an HSV virus during a subsequent challenge.

Methods for measuring the dose of an immunogen (e.g. in human subjects) are well known in the art, and include, for example, dose-escalating trials.

The recombinant HSV proteins of the invention may be used for the improvement of an existing HSV-1 or HSV-2 vaccine.

In some embodiments, any of the HSV vaccines of and for use in the methods of this invention will comprise an HSV protein or combination of HSV proteins of the present invention, in any form or embodiment as described herein. In some embodiments, any of the HSV vaccines of and for use in the methods will consist of an HSV protein or combination of HSV proteins of the present invention, in any form or embodiment as described herein. In some embodiments, the HSV vaccines of this invention will consist essentially of an HSV protein or combination of HSV proteins of the present invention, in any form or embodiment as described herein. In some embodiments, the term “comprise” refers to the inclusion of other recombinant HSV proteins, as well as inclusion of other proteins that may be known in the art. In some embodiments, the term “consisting essentially of” refers to a vaccine, which has the specific HSV protein or fragment thereof. However, other peptides may be included that are not involved directly in the utility of the HSV protein(s). In some embodiments, the term “consisting” refers to a vaccine having a particular HSV protein or fragment or combination of HSV proteins or fragments of the present invention, in any form or embodiment as described herein.

The compositions of the invention may be used for preventing HSV-1, HSV-2 or a symptom or manifestation thereof, the composition comprising a vaccine of the present invention.

In another embodiment, of methods of the present invention, a vaccine of the present invention is administered as a single inoculation. In another embodiment, the vaccine is administered twice, three times or four times or more.

In another embodiment, the vaccine is administered at separate sites with gD separate from gC. In another embodiment, the vaccine is administered at 1, 2, 3 or 4 week intervals. In another embodiment, the vaccine is administered at 1, 2, 3, 4, 5, or 6 month intervals or any combination thereof. An exemplary 3 dose vaccine may be given at months 0, 2 and 6.

### Formulations

The pharmaceutical compositions containing the HSV recombinant proteins can be administered to a subject by one or more method known to a person skilled in the art, such as parenterally, transmucosally, transdermally, intramuscularly, intravenously, intra-dermally, intra-nasally, subcutaneously, intra-peritonealy, intra-ventricularly, intra-cranially, or intra-vaginally.

In another embodiment, vaccines of the instant invention are administered via epidermal injection, intramuscular injection, subcutaneous injection, or intra-respiratory mucosal injection.

In another embodiment, the pharmaceutical compositions are administered by intravenous, intra-arterial, or intra-muscular injection of a liquid preparation. Liquid formulations include solutions, suspensions, dispersions, emulsions, oils and the like. In another embodiment, the pharmaceutical compositions are administered intravenously and are thus formulated in a form suitable for intravenous administration. In another embodiment, the pharmaceutical compositions are administered intra-arterially and are thus formulated in a form suitable for intra-arterial administration. In another embodiment, the pharmaceutical compositions are administered intra-muscularly and are thus formulated in a form suitable for intra-muscular administration.

In another embodiment, pharmaceutical compositions are administered orally, and are thus formulated in a form suitable for oral administration, i.e., as a solid or a liquid preparation. Solid oral formulations include tablets, capsules, pills, granules, pellets and the like. Liquid oral formulations include solutions, suspensions, dispersions, emulsions, oils and the like.

In other embodiments, pharmaceutically acceptable carriers for liquid formulations are aqueous or non-aqueous solutions, suspensions, emulsions or oils. Examples of nonaqueous solvents are propylene glycol, polyethylene glycol, and injectable organic esters such as ethyl oleate. Aqueous carriers include water, alcoholic/aqueous solutions, emulsions or suspensions, including saline and buffered media. Examples of oils are those of animal, vegetable, or synthetic origin, for example, peanut oil, soybean oil, olive oil, sunflower oil, fish-liver oil, another marine oil, or a lipid from milk or eggs.

In another embodiment, parenteral vehicles (for subcutaneous, intravenous, intraarterial, or intramuscular injection) include sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Examples are sterile liquids such as water and oils, with or without the addition of a surfactant and other pharmaceutically acceptable adjuvants. In general, water, saline, aqueous dextrose and related sugar solutions, and glycols such as propylene glycols or polyethylene glycol are preferred liquid carriers, particularly for injectable solutions. Examples of oils are those of animal, vegetable, or synthetic origin, for example, peanut oil, soybean oil, olive oil, sunflower oil, fish-liver oil, another marine oil, or a lipid from milk or eggs.

In another embodiment, the pharmaceutical composition is delivered in a controlled release system. For example, the agent can be administered using intravenous infusion, an implantable osmotic pump, a transdermal patch, liposomes, or other modes of administration. In another embodiment, a pump is used (Sefton, 1987, CRC Crit. Ref. Biomed. Eng. 14:201; Buchwald et al., 1980, Surgery 88:507; Saudek et al., 1989, N. Engl. J. Med. 321:574. In another embodiment, polymeric materials are used; e.g. in microspheres in or an implant.

Also comprehended by the invention are compounds modified by the covalent attachment of water-soluble polymers such as polyethylene glycol, copolymers of polyethylene glycol and polypropylene glycol, carboxymethyl cellulose, dextran, polyvinyl alcohol, polyvinylpyrrolidone or polyproline. The modified compounds are known to exhibit substantially longer half-lives in blood following intravenous injection than do the corresponding unmodified compounds. Such modifications also increase, in another embodiment, the compound's solubility in aqueous solution, eliminate aggregation, enhance the physical and chemical stability of the compound, and greatly reduce the immunogenicity and reactivity of the compound. In another embodiment, the desired in vivo biological activity is achieved by the administration of such polymer-compound abducts less frequently or in lower doses than with the unmodified compound.

An active component is, in another embodiment, formulated into the composition as neutralized pharmaceutically acceptable salt forms. Pharmaceutically acceptable salts include the acid addition salts (formed with the free amino groups of the polypeptide or antibody molecule), which are formed with inorganic acids such as, for example, hydrochloric or phosphoric acids, or such organic acids as acetic, oxalic, tartaric, mandelic, and the like. Salts formed from the free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

Having described preferred embodiments of the invention with reference to the accompanying drawings, it is to be understood that the invention is not limited to those precise embodiments, and that various changes and modifications may be effected therein by one skilled in the art without departing from the scope or spirit of the invention as defined in the appended claims.

The following examples illustrate, but do not limit the invention.

### EXAMPLES

### Example 1

### Construction of the Vectors for Production of Recombinant HSV Glycoproteins

DNA sequences encoding two different versions of the HSV-2 G strain gD protein ectodomain were synthesized and cloned and named pGLY2757 and pGLY2758 (GeneArt, Inc., Toronto, CA). The two constructs differed by the length of the C-terminus, one encoding the entire ectodomain, amino acids 26-339 (gD 339, pGLY2757) and a second encoding a shorter version without the C-terminal domain, amino acids 26-306 and including two heterologous amino acids, Asn and Gln, appended to the C-terminus after Leu 306 (gD 306NQ, pGLY2758). Both constructs also included a Gly3His9 C-terminal histidine tag. Each of these plasmids was subcloned into a P. pastoris expression vector containing the AOX1 promoter and ShBLE drug resistance marker and fused in frame with the S. cerevisiae α-Mating Factor pre secretion signal and named pGLY2960 and pGLY2961, respectively.

A DNA sequence encoding a third version of the HSV-2 G strain gD protein ectodomain (amino acids 26-310) and including a Gly3His9 C-terminal histidine tag (G3H9), was generated by PCR using primers RCD738 (GAATTCGAAACGATGAGATTTCCTTC SEQ ID NO: 17) and RCD810 (GGCCGGCCCTATTAGTGATGGTGGTGGTGATGGTGATGATGACCAC CACCAGTACCAGCTGGATCTTCCAACAAAGCGG SEQ ID NO: 18) and cloned into the Topo TA cloning vector (Invitrogen, Carlsbad, Calif.), and named pGLY4717. The gD 310 sequence was subcloned into a P. pastoris expression vector containing the AOX1 promoter and ShBLE drug resistance marker and fused in frame with the S. cerevisiae α-Mating Factor pre secretion signal and named pGLY4722.

A DNA sequence encoding the HSV-2 G strain gC protein ectodomain (amino acids 24-448) where amino acids 445-448 were mutated to a Factor Xa protease recognition motif (IEGR) and including a C-terminal (G3H9) Histidine tag, was synthesized and cloned and named pGLY3640 (GeneArt, Inc., Toronto, CA). The DNA sequence from this plasmid was subcloned into a P. pastoris expression vector containing the AOX1 promoter and ShBLE drug resistance marker and fused in frame with the S. cerevisiae α-Mating Factor prepro secretion signal and named pGLY3653.

A second DNA sequence encoding a second HSV-2 G strain gC protein ectodomain (amino acids 24-448) including a N-terminal histidine tag (MGH9G3) followed by a Factor Xa protease recognition motif (IEGR), was generated by PCR using primers RCD856 (GAGTCAAAAAATGGGTCATCATCATCACCATCACCACCATCACGGTGGTGGTATCGAAGGTAGA TTGGCTAATGCTTCTCCAGGTAGAAC SEQ ID NO: 19) and RCD857 (GGCCGGCCCTTATTAAGCACCTTCAACAGCTCTGATAACCTGTCTCTCAG SEQ ID NO: 20) and pGLY3653 as a template DNA and then cloned into the Topo TA cloning vector (Invitrogen, Carlsbad, Calif.), and named pGLY4744. The DNA sequence from pGLY4744 was subcloned into a P. pastoris expression vector containing the AOX1 promoter and ShBLE drug resistance marker and fused in frame with the S. cerevisiae α-Mating Factor prepro secretion signal and named pGLY4749.

A third DNA sequence encoding a second HSV-2 G strain gC protein ectodomain (amino acids 75-448) including a N-terminal histidine tag (H9G3) and mutation of the last four amino acids from VEGA to a Factor Xa protease recognition motif (IEGR), was generated by PCR using primers RCD904 (GAGTCAAAAACATCATCATCACCATCACCACCATCACGGTGGTGGTACTGCTAAACCAGCTCCA CCACC SEQ ID NO: 21) and RCD905 (GAGTCAAAAAACTGCTAAACCAGCTCCACCACC SEQ ID NO: 22) and pGLY3640 as a DNA template, and then cloned into the Topo TA cloning vector (Invitrogen, Carlsbad, Calif.), and named pGLY5623. The DNA sequence from pGLY5623 was subcloned into a P. pastoris expression vector containing the AOX1 promoter and ShBLE drug resistance marker and fused in frame with the S. cerevisiae α-Mating Factor prepro secretion signal and named pGLY5625.

A DNA sequence encoding the HSV-2 gE protein ectodomain (amino acids 24-406) including a Factor Xa protease recognition motif (IEGR) and a C-terminal (G3H9) Histidine tag, was synthesized and cloned and named pGLY4688 (GeneArt, Inc., Toronto, CA). The DNA sequence from pGLY4688 was subcloned into a P. pastoris expression vector containing the AOX1 promoter and ShBLE drug resistance marker and fused in frame with the S. cerevisiae α-Mating Factor prepro secretion signal and named pGLY4756.

A DNA sequence encoding the HSV-2 gB protein ectodomain (amino acids 23-719) including an N-terminal (H9G3) Histidine tag, was synthesized and cloned and named pGLY3715 (GeneArt, Inc., Toronto, CA). The DNA sequence from pGLY3715 was subcloned into a P. pastoris expression vector containing the AOX1 promoter and ShBLE drug resistance marker and fused in frame with the S. cerevisiae α-Mating Factor prepro secretion signal and named pGLY4750. Each of the DNA sequences encoding a HSV glycoprotein was codon optimized for high level expression in yeast. See Table 2 for some of the sequences used.

### Example 2

### Expression of Recombinant HSV Viral Glycoproteins in Yeast

This example describes how to produce HSV viral glycoproteins in yeast host cells. It also demonstrates that expression of HSV-2 gC in Pichia pastoris was enhanced by using host cells in which the gene encoding the endogenous PDI1 has been inactivated and replaced with an expression cassette encoding the human PDI.

### Transformation

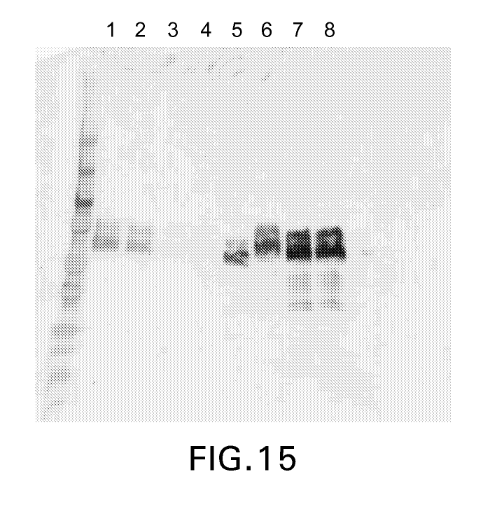

P. pastoris yeast strains were transformed with HSV viral glycoprotein expression vectors by electroporation (using standard techniques as recommended by the manufacturer of the electroporator, BioRad). As an example, yeast strains YGLY733, YGLY3625 and YGLY3626 were transformed with the HSV-2 gC expression construct pGLY3653. Strain YGLY733 is a GFI2.0 strain which PDI does not express any human chaperones. Strains YGLY3625 and YGLY3626 are GFI2.0 strains which harbor deletions of the P. pastoris PDI gene and express a copy of the human PDI gene. Colonies were selected on standard Pichia rich medium plates (Invitrogen) containing 150 mg/L of Zeocin (Invitrogen). Clones were cultivated in standard baffled shake flasks for 72 h at 25° C. in standard BMGY liquid rich medium (Invitrogen) containing glycerol as the sole carbon source, then pelleted and resuspended in BMMY rich medium containing methanol as the sole carbon source and cultivated an additional 24 h. The cultures were pelleted and the supernatants analyzed for expression of gC by Western blot using the anti-HIS mAb (Santa Cruz). Western analysis shows that strain YGLY733 expresses significantly less gC than strains YGLY3625 or YGLY3626, both of which have deletions for P. pastoris PDI and express human PDI (FIG. 15).

### Fermentation Process Description

Fermentation runs were carried out in 15 L (12 L working volume) autoclavable glass bioreactors from Applikon or in 0.5 L (300 ml working volume) autoclavable glass bioreactors from Infors. The reactor is inoculated (0.04% v/v) with an exponential phase shake flask culture grown from a frozen stock vial. The batch phase ends in 24-36 hours upon depletion of the initial charge glycerol. The wet cell weight (WCW) after the batch phase is typically 120±25 g/L WCW. At this point a 50% w/w glycerol solution containing 12 mL/L PTM1 salts is fed to the fermentor in a single pulse leading to a final glycerol concentration of 30 g/L at the start of the glycerol fed-batch phase. A solution containing a synthetic inhibitor of fungal O-glycosylation (PMTi-3) dissolved in methanol at 2.6 mg/mL is added at 1 mL/L. A protease inhibitor cocktail (45 mg/mL Pepstatin A and 15 mM of Chymostatin in DMSO) is added at 0.6 mL/L. Within 4 hours the glycerol is consumed and the wet cell weight has reached 225±25 g/L WCW. Gene expression is then induced by the initiation of a methanol feed containing 12 mL/L of PMT1 salts at 2.3 g/h/L. At the start of the methanol feed batch phase as well as every 24 hours of induction, 1 mL/L of 2.6 mg/mL PMTi-3 in methanol and 0.6 mL/L of the protease inhibitor cocktail are added. Induction continues for 40 hours when the final wet cell weight is expected to be approximately 300±25 g/L. (L* is the initial charge volume before inoculation).

Primary clarification of fermentor broth is performed by centrifugation. The whole cell broth is transferred into 1000 mL centrifuge bottles and centrifuged at 4° C. for 15 minutes at 13,000×g.

### Purification

HIS-tagged protein is purified by standard metal affinity chromatography using an AKTA or AKTA pilot (GE biosciences) and streamline chelating packed on a K9/15 column 0.9×13 cm, GE biosciences. See Hamilton et al., 2006, Science 313:1441-1443.

Analysis of N-Glycans from Viral Glycoproteins

Representative samples of recombinant HSV glycoproteins were assayed for their purity using MALDI-TOF. The protein was deglycosylated by the addition of 30 μl of 10 mM NH₄HCO₃ pH 8.3 containing one milliunit of N-glycanase (Glyko). After 16 hr at 37° C., the solution containing the glycans was removed by centrifugation and evaporated to dryness.

Molecular weights of the glycans were determined using a Voyager DE PRO linear MALDI-TOF (Applied Biosciences) mass spectrometer as described previously (Choi et al., 2003, Proc Natl Acad Sci USA 100:7022-7027).

For N-glycan quantification, enzymatically released N-glycans were then labeled with 2-aminobenzidine (2-AB) using a commercial 2-AB labeling kit (Prozyme, San Leandro, Calif.). HPLC was performed using a Prevail CHO ES, 5 micron bead, amino-bound silica column maintained at 30° C. N-glycan species determinations were then made based on commercial and in-house prepared standards and MALDI-TOF mass spectrometer mass measurements (Table 3).

TABLE 3
Purity of N-glycans on HSV glycoproteins expressed in Pichia

| PROTEIN | LOT # | GLYCOFORM | %-PURITY |
|---|---|---|---|
| gC | 1 | 2.0 | 88 |
| gC | 2 | 2.0 | 78 |
| gC | 3 | 2.0 | 95 |
| gC | 4 | 2.0 | 95 |
| gD 339 | 1 | 2.0 | 81.5 |
| gD 339 | 2 | 2.0 | 85 |
| gD 339CC | 1 | 2.0 | 88 |
| gD 310 | 1 | 2.0 | 88 |
| gD 339 | 1 | 5.9 | 71 |
| gD 339 | 1 | 6.0 | 58 |

The average purity of glycoforms in strain 2.0 was greater than 87%. Only single samples from strain 5.9 and 6.0 were assayed. The average purity in these strains was 71% and 58%, respectively.

### Example 3

### Efficacy of a Soluble gC-2 Vaccine in the Guinea Pig Vaginal Model of HSV-2 Infection

Subunit vaccines consisting of glycoproteins essential for viral entry have demonstrated limited efficacy in humans; indicating that exploring alternative strategies would be beneficial. gC is a virulence factor that mediates immune evasion by binding to C3b, inhibiting complement-mediated neutralization. Studies in mice vaccinated with a soluble form of HSV-1 gC show reduced disease severity upon HSV-1 challenge despite a lack of neutralizing antibodies to gC.

Guinea pigs were immunized with the ectodomain of HSV-2 gC (gCt-2) encompassing AA 24-444 to test for protection from disease upon HSV-2 challenge, akin to the HSV-1 model. The gCt-2 proteins used for immunizations were purified from the fungal yeast, Pichia pastoris, which had been genetically engineered to express glycoproteins with humanized N-glycosylation structures as described in Example 2. See Hamilton et al., 2003, Science 301:1244-1246; and Choi et al., 2003, Proc Natl Acad Sci USA 100:5022-5027.

### Structure-Function Analysis

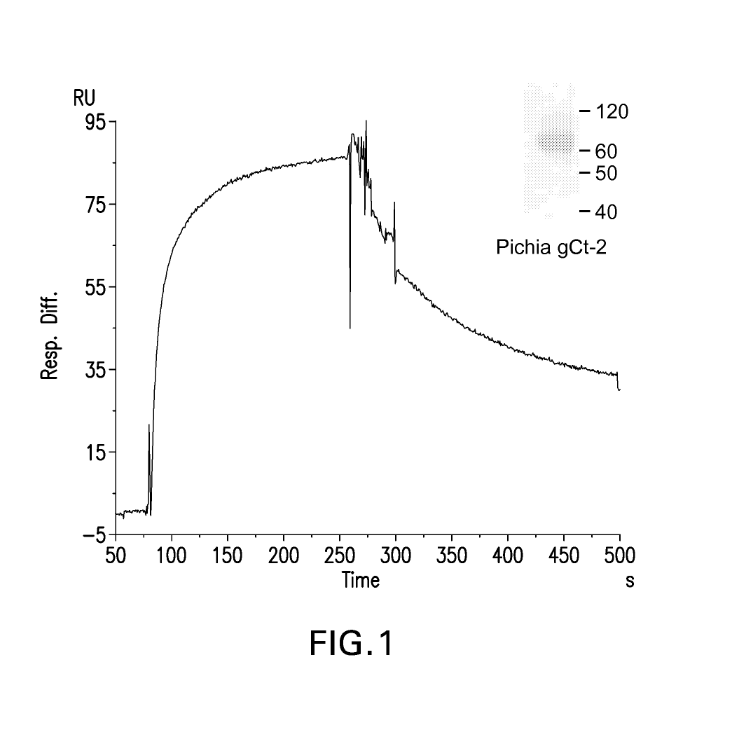

Seven hundred fifty nanograms of gCt-2 (2.0) was resolved by SDS-PAGE, transferred to nitrocellulose and then Western blotted with a rabbit polyclonal antiserum recognizing gC using standard methods (Hung et al., 1992, J. Virol. 66:4013-4027) (R81; provided by Roselyn Eisenberg and Gary Cohen, University of Pennsylvania). Results are shown in the FIG. 1 insert. The predicted molecular weight of gCt-2, 57 kDa, included estimating the mass of the 7 N-linked glycans. The blot shows the majority of protein is slightly larger than the predicted size. The observed differences from the predicted size are due to the high degree of O-glycosylation, especially at the amino terminus of gCt-2 (Olofsson, et al., 1981, J. Virol. 38:564-570).

To evaluate function of gCt-2, human complement protein, C3b, was biotinylated with sulfo-N-hydroxysuccinimide-biotin (Pierce) by primary amine coupling per manufacturer's instructions. The average number of biotin molecules per gC was 1.78 and was determined per manufacturer's instructions. Using a BIACORE 2000 at room temperature, according to the manufacturer's protocols, biotinylated C3b was immobilized at high density (770 response units [RU]) on a carboxylmethyldextran chip coated with streptavidin (SA-CM5). The running buffer was HBS (10 mM HEPES, 150 mM NaCl, pH 7.4) containing 0.005% P-20 (surfactant). Glycoprotein C proteins at a concentration of 10 μg/ml were flowed over the chip's surface at 30 μl/min for 3 min. Buffer was flowed over the chip for 3 min to examine dissociation of gC from C3b. Soluble gD, which served as a negative control because it does not bind C3b, was flowed over the chip and shown not to bind to the C3b chip (data not shown). Binding curves of gCt-2 proteins were corrected for non-specific binding to the C3b-CM5 chip by subtracting flow response over a blank SA-CM5 flow cell (FC1) from the flow response over the C3b-coated flow cell (FC2). By Biacore analysis, gCt-2 protein bound to human C3b (See FIG. 1). Specifically, gCt-2 bound to C3b with rapid kinetics and dissociated with reduced kinetics relative to association. These results are consistent with a gCt protein of similar size expressed from baculovirus (Rux et al., 2002, Virology 294: 324-332).

### Vaccine Efficacy

Female Hartley guinea pigs weighing between 200 and 250 g were vaccinated IM in the hind flank on days 0, 7, and 21 with 0, 10, 25, 50, or 100 μg of soluble, HSV-2 gCt-2 (2.0) adjuvanted with Merck Aluminum Adjuvant (MAA). On day 42 post-initial vaccination (3 weeks post-final vaccination), animals were anesthetized and serum samples were collected via the superior vena cava. On day 43, guinea pigs were anesthetized, swabbed vaginally three times to clear the vaginal tract and inoculated with 5×10⁵ PFU of HSV-2, strain G. Vaginal swab samples were collected daily between days 44 and 57 post-initial vaccination (1 and 14 d post-infection [p.i.]). Guinea pigs were observed through day 64 post-initial vaccination (day 21 p.i.) for HSV-2 disease. On days 112 and 113 d post-initial vaccination (69 and 70 p.i.), lumbosacral dorsal root ganglia (DRG) were extracted from challenge survivors for real-time PCR analysis.

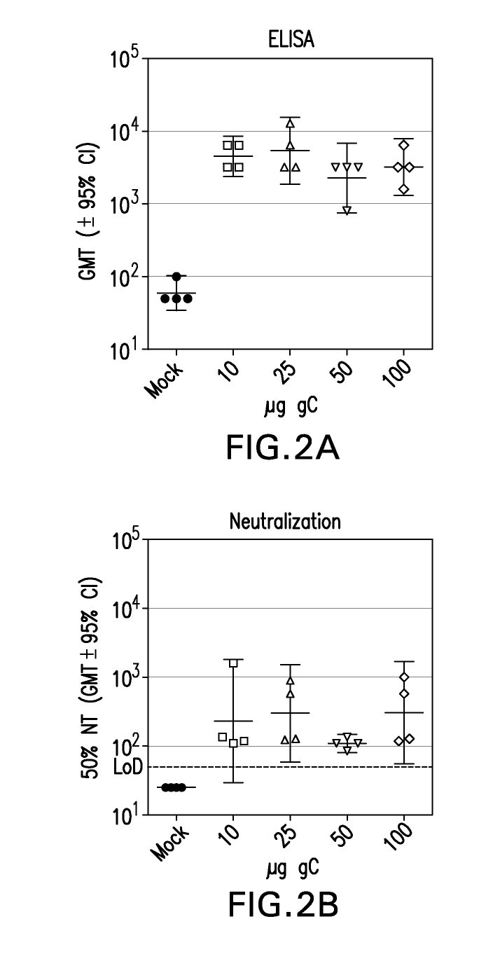

To evaluate the levels of total IgG produced against HSV-2 gC, Immulon 4B microtiter plates were coated overnight at 4° C. with 50 ng/well of purified HSV-2 lysate in bicarbonate buffer. The plates were blocked the following day with 1% BSA and 0.05% Tween 20 in PBS, and serial dilutions of guinea pig sera were added to the wells. The plates were washed to remove any unbound antibody and bound IgG was detected using horseradish peroxidase-conjugated secondary antibody and TMB substrate and read at an OD of 405 nm with a Molecular Devices VERSAmax variable wavelength plate reader. ELISA geometric mean endpoint titers to HSV-2 lysate were plotted as the reciprocal dilution of the endpoint (FIG. 2A). Pichia gCt-2 elicited circulating IgG to HSV-2 that was significantly higher than Mock (P>0.04, Mann-Whitney test). There was no significant difference, however, in levels of serum IgG as a function of antigen concentration (P>0.31) indicating that there was no dosing effect.

Neutralization titers were evaluated using ELVIS® cells (Diagnostic HYBRIDS), which are a BHK cell line stably transfected with lacZ driven by the HSV-1 UL39 promoter. Upon HSV infection of ELVIS® cells, viral immediate-early genes induce the UL39-LacZ cassette. Guinea pig sera was diluted in culture media containing 6.7% rabbit complement and co-incubated with 1,800 PFU of HSV-2 strain MS for 1 h at 37° C. Antibody/virus mix was subsequently added to the ELVIS® cell monolayer and cultured overnight. β-galactosidase production was measured using a Gal-Screen kit (Applied Biosystems). The 50% neutralization titer represents the dilution of serum at which β-galactosidase levels are 50% that of the positive control (HSV-2 in the absence of serum). The geometric mean 50% neutralization titers are plotted as the reciprocal dilution (FIG. 2B). gCt-2 elicited neutralizing antibodies. Similar to the ELISA results, there was no significant differences in levels of neutralizing antibodies as a function of gCt-2 concentration (P>0.11).

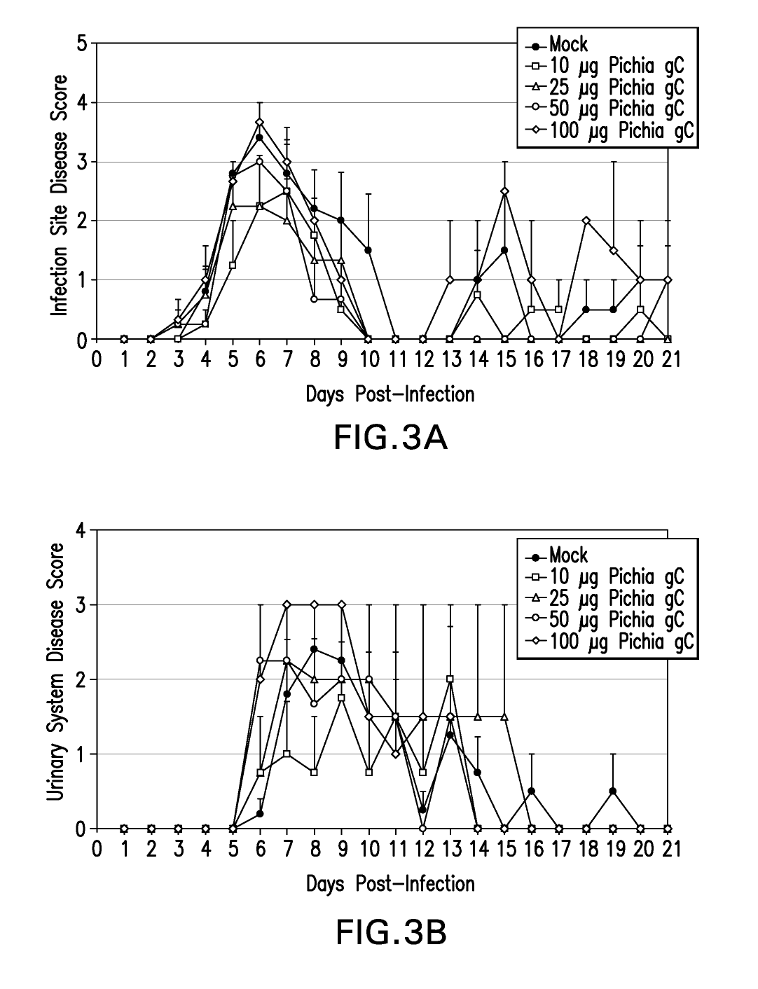

To evaluate the extent of HSV-2 vaginal disease, a scoring system was adapted from Stanberry et al., 1982 (Stanberry et al., 1982, J Infect Dis 146:397-404), whereby infected animals were given a score of 0 to 4. Guinea pigs given a score of 0 exhibited no signs of HSV infection. A score of 1 denoted redness and/or swelling of the perineum. Animals with a score of 2 exhibited 1 or 2 herpetic lesions or had 1 or more healing lesions on the perineum whereas those with a score of 3 had 3 or more lesions. Guinea pigs that had a score of 4 exhibited coalesced or ulcerated lesions on the perineum. The results for each vaccine group were plotted as the mean+standard error of the mean (FIG. 3A). Urinary tract disease was also scored. A score of 1 denoted excretion of apparent purulent exudate during urination. A score of 2 was given to animals with urinary retention. Guinea pigs with a score of 3 expressed bloody urine. Notably, severe hemorrhagic cystitis was the primary cause of removal of animals from the study. The results for each vaccine group are plotted as the mean+standard error of the mean (FIG. 3B).

Mock-vaccinated animals exhibited severe infection site and urinary system disease. During the acute phase of infection, these animals presented with edema, erythema, and herpetic lesions on the perineum between days 4 and 10 p.i. and acute urinary retention with purulent exudate and blood containing urine between days 7 and 14 p.i. Vaccination with low concentrations of gCt-2 partially alleviated disease symptoms, but the reductions were not significant (P>0.17, Two-sided t test). Animals that received higher concentrations of gCt-2 exhibited more severe disease symptoms than those that received 10 μg of gCt-2. Notably, disease scores in animals vaccinated with 100 μg of Pichia gCt-2 were higher than Mock on many days post-challenge.

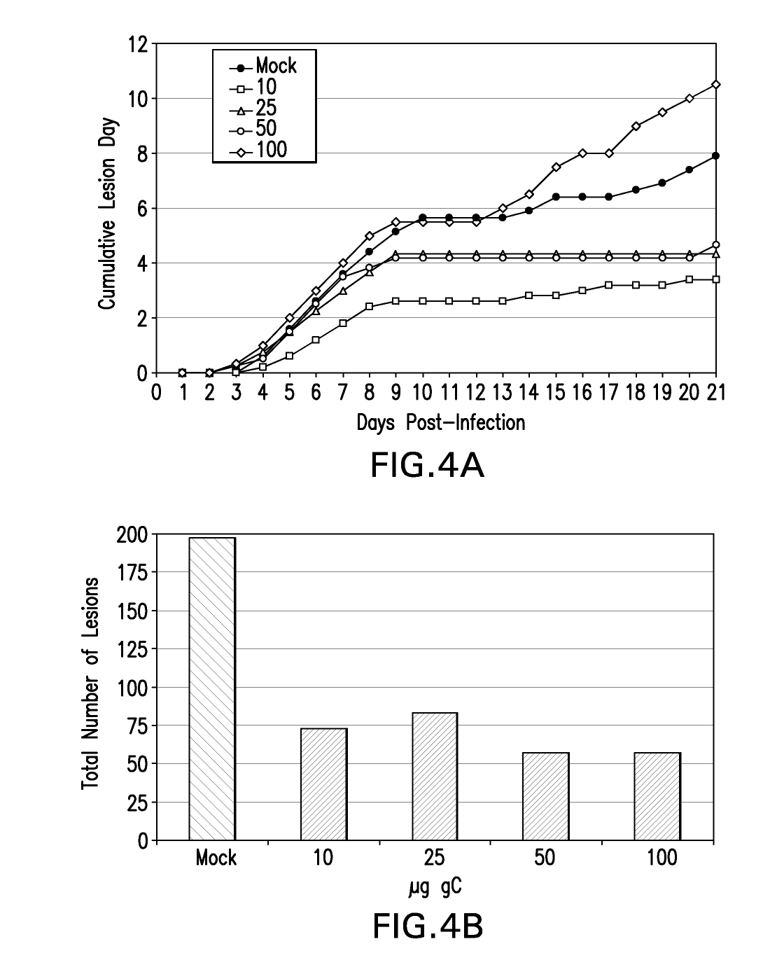

Lesion days, which are the days when clinically apparent herpetic lesions were observed on the perineum, were plotted cumulatively per day for 21 days post-infection. In addition, the total number of lesions on the perineum of HSV-2-infected guinea pigs were quantified for 21 days and summed per vaccine group. With the exception of the 100 μg Pichia group, all vaccinated groups had reduced lesion days relative to Mock (FIG. 4A). Only animals vaccinated with 10 μg of gCt-2 exhibited reduced kinetics of lesion days. Animals vaccinated with 25 μg or more of gCt-2 had kinetics of lesion days similar to mock-vaccinated animals. Notably, animals vaccinated with 100 μg gCt-2 had more rapid kinetics than Mock during both the acute (days 3 to 14) and recurrence phases (days 14 to 21) of infection.

The overall number of lesions was reduced in all gCt-2 vaccinated groups relative to Mock (FIG. 4B). Vaccination with gCt-2 reduced the total number of lesions by 2.4- to 2.8-fold relative to Mock. Together, these results show that vaccination with gCt-2 reduces the frequency and the total number of lesions exhibited during HSV-2 infection.

To evaluate the levels of HSV-2 shed into the vaginal vault, the vaginas of guinea pigs were swabbed daily for 14 days p.i. Vaginal swab samples were frozen at −80° C. in tissue culture medium. Frozen samples were later thawed and thoroughly mixed, and infectious virus was quantified by standard plaque assays on Vero cell monolayers. Swab samples were titrated on Vero cell monolayers by standard plaque assay (level of detection of 0.7 log₁₀ PFU/vagina). Viral titers per swab were determined and plotted as the mean+standard error of the mean.

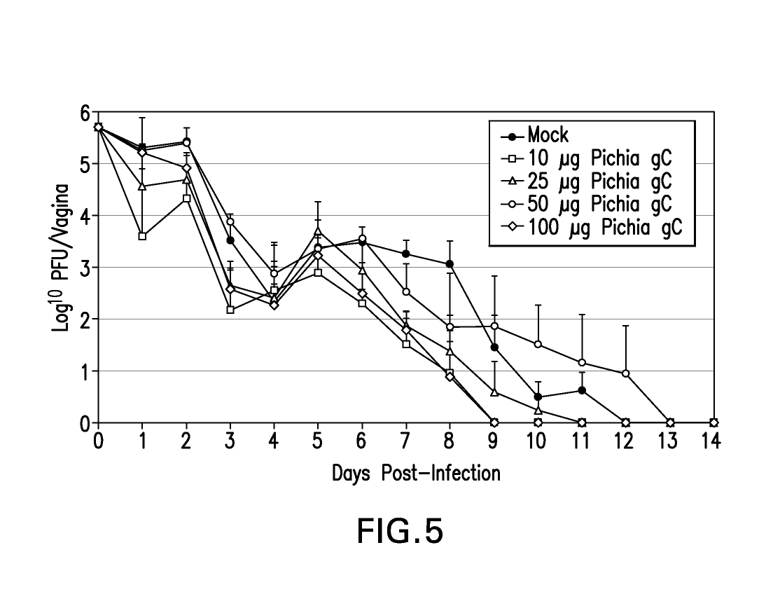

In Mock animals, HSV-2 peaked on days 2 and 6 p.i. and was not detected after day 11 p.i. (FIG. 5). Animals vaccinated with 10, 25, or 100 μg of gCt-2 showed peak titers on days 2 and 5 p.i. and virus was not detected in vaginal secretions between days 9 and 11 p.i., 1 to 3 days shorter than Mock. Specifically, the levels of virus in vaginas was significantly lower than Mock on days 7 and 8 p.i. for the 10 μg group (P=0.03 for both days, two-sided t test), day 7 p.i. for the 25 μg group (P=0.01), and days 6 and 7 for the 100 μg group (P=0.04 and 0.01, respectively). Animals vaccinated with 50 μg of gCt-2 had virus levels similar to Mock. Notably, both Pichia 10 μg and 100 μg groups had similar levels of virus in the vagina between days 3 and 14 p.i. yet they represented the extremes in disease severity with 100 μg gCt-2 animals exhibiting the most severe herpetic disease and the 10 μg gCt-2 animals exhibiting the least amount of disease (see FIG. 3). The two results together suggest that there is a lack of correlation between viral loads in the vagina and the severity of disease.

To determine the efficiency of the establishment of latency in vaccinated animals, surviving guinea pigs were euthanized on days 69 and 70 p.i. and lumbosacral DRG were harvested. Total DNA was isolated from DRG using the Qiamp DNA mini kit (Qiagen, Hilden, Germany) as per the manufacturer's instructions. HSV-2 DNA was quantified in duplicate using a primer/probe set to the gG gene (US4) and normalized to the host cell gene, GAPDH. Quadruplet standard curves were constructed from 10-fold dilutions of gG-2 plasmid and purified guinea pig DNA (BioChain Institute, Inc.). The DNA sample along with oligonucleotide primer/probe sets specific for gG-2 and GAPDH were mixed with Quantitect Multiplex PCR no Rox Master Mix (Qiagen). Reactions were carried out in a Stratagene Mx3005P Real Time PCR system and analyzed with Stratagene MxPro Software. The results are plotted as the copy number of HSV-2 DNA per 10⁶ copies of GAPDH. Samples that had no detectable viral genomes were given a value that was 50% of the LoD (26 genome copies).

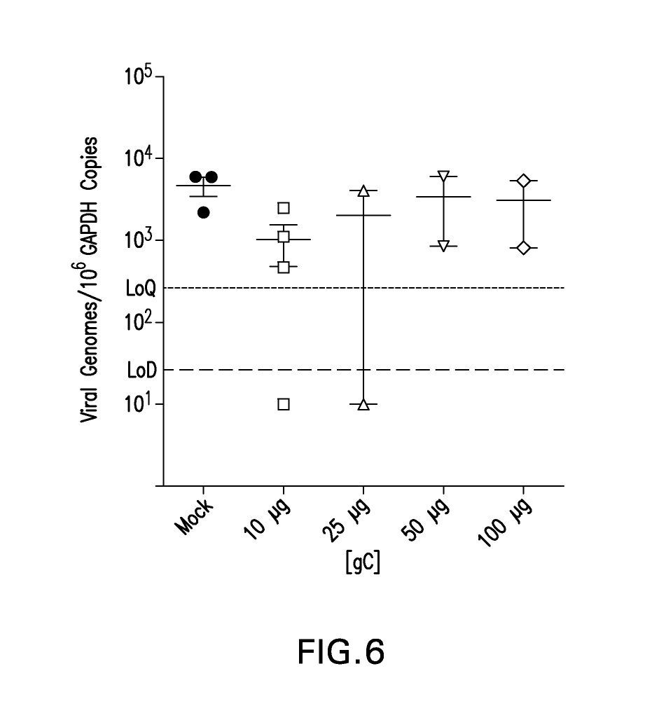

The results showed that animals vaccinated with 10 μg of gCt-2 had levels of viral genomes 4.6-fold lower than Mock (FIG. 6). This was the only group where viral genome levels were significantly reduced compared to Mock (P=0.03, Two-sided t test). Relative to Mock, animals vaccinated with 25, 50 or 100 μg of gCt-2 exhibited 2.4-, 1.3- and 1.5-fold reductions in viral genomes. These results suggest an inverse relationship between the amount of gCt-2 used to vaccinate and the level of challenge virus genomes in DRG. Together, these results indicate that gCt-2 vaccination only modestly reduces viral genome loads in lumbosacral DRG.

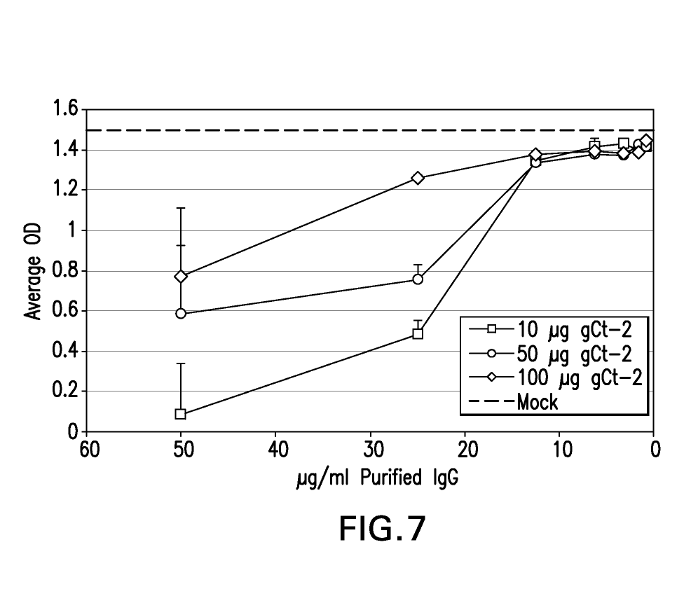

Because of the inhibitory activity that complement protein C3b has on virus replication, the levels of antibody that block gC:C3b interactions in sera from vaccinated guinea pigs was measured in an ELISA format. Purified human C3b (Complement Technologies) at 200 ng/well in bicarbonate buffer was coated overnight at 4° C. on Immulon 4B microtiter plates. The plates were blocked the following day. Simultaneous to the blocking of C3b-coated plates, serial dilutions of purified IgG from gCt-2-immunized guinea pig sera was mixed with 50 ng of gCt-2 and incubated for 1 h at 37° C. with gentle mixing. The IgG/gC mix was transferred to the C3b-coated plates and incubated for 1 h at room temperature. The plates were washed to remove any unbound gC. Glycoprotein C bound to C3b was detected at an OD of 405 nm using rabbit polyclonal antibody R81 and anti-rabbit IgG HRP. The results are plotted as the OD405 per μg/ml of antibody (FIG. 7).

The results showed that dose-dependent blocking of gCt-2 binding to immobilized C3b was observed in all groups. IgG from animals vaccinated with 10 μg of gCt-2 had the highest C3b-blocking activity followed by 50 μg and 100 μg of gCt-2. This result shows an inverse correlation between the amount of gCt-2 used for vaccination and the levels of IgG that block binding of gC to C3b. Moreover, there is a correlation between the C3b blocking activity and severity of HSV-2 disease (compare FIG. 7 with FIG. 3). Thus, guinea pigs vaccinated with gCt-2 elicit IgG that blocks gC-C3b interactions.

The overall summary of the results suggest that gC-2 is not efficacious in inhibiting viral disease in guinea pigs but can reduce viral titers at the site of infection as well as the level of viral genomes in the DRG. Moreover, the results suggest a correlation between vaccine efficacy and the production of antibodies that block C3b binding to HSV-2 gC.

### Example 4

### Efficacy of a Soluble gD-2 Vaccine in the Mouse Vaginal Model of HSV-2 Infection

The full length ectodomain of HSV-2 gD encompassing AA 26-339 was purified from media of Pichia strains 2.0, 5.9, and 6.0 by immobilized metal affinity chromatography as described in Example 2. To test whether these proteins were correctly folded, the biological activity was tested by determining whether the viral glycoproteins could block HSV-2 infection by binding to receptors on permissive cells.

Vero cell monolayers were pretreated with serial dilutions of gD-2 for 1 h at 4° C. Baculovirus 331 (formally termed 306: Nicola et al., J. Virol. 70:3815, 1996) was included because this protein has been evaluated in this assay and the results were published and referenced. The negative control was untreated cells. Approximately 100 PFU of HSV-2 US5::lacZ virus was added to each well and incubated for 1 h at 4° C. The inoculum was replaced with fresh medium containing 0.5% methyl cellulose and incubated for 18 h at 34° C. (both the methyl cellulose and 34° C. incubation were used to minimize spread of virus). Cells were stained the following day, and βgal⁺ cells were counted.

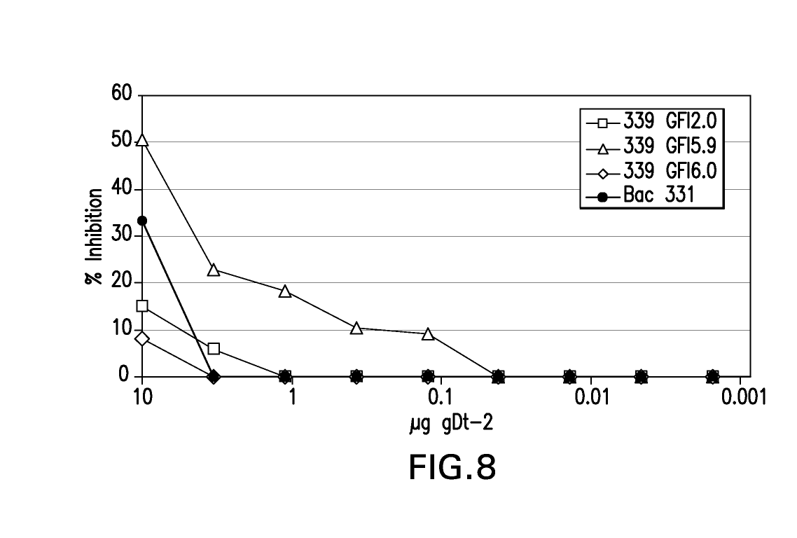

The results showed a dose-dependent inhibition of HSV-2 infection (FIG. 8). Of the three gDt-2 339 proteins, the protein derived from the GFI5.9 strain was most active. Protein derived from the GFI2.0 strain was less active in the range of concentrations tested. Protein from the GFI6.0 strain was the least active. Bac 331 had an intermediate activity between OFT 5.9 and 2.0 proteins

Vaccination and HSV-2 Challenge of Mice with Various Forms of gDt-2 Produced from the GFI2.0, 5.9, and 6.0 Strains of Pichia pastoris

Six week old female BALB/c mice were vaccinated on days 0, 6, and 20 with either 1 or 3 μg of GlycoFi Pichia-expressed gDt-2 310 (GFI2.0) or 339 (GFI 2.0, 5.9 or 6.0) or Bac 331. Notably, no adjuvant was used in this test. Animals were bled on day 35 and challenged by intravaginal inoculation with 5×10⁵ PFU of HSV-2 strain G. Mice were evaluated for end point antibody titers against lysates of purified HSV-2 strain G and for protection from death because of vaccine-induced neutralizing antibodies.

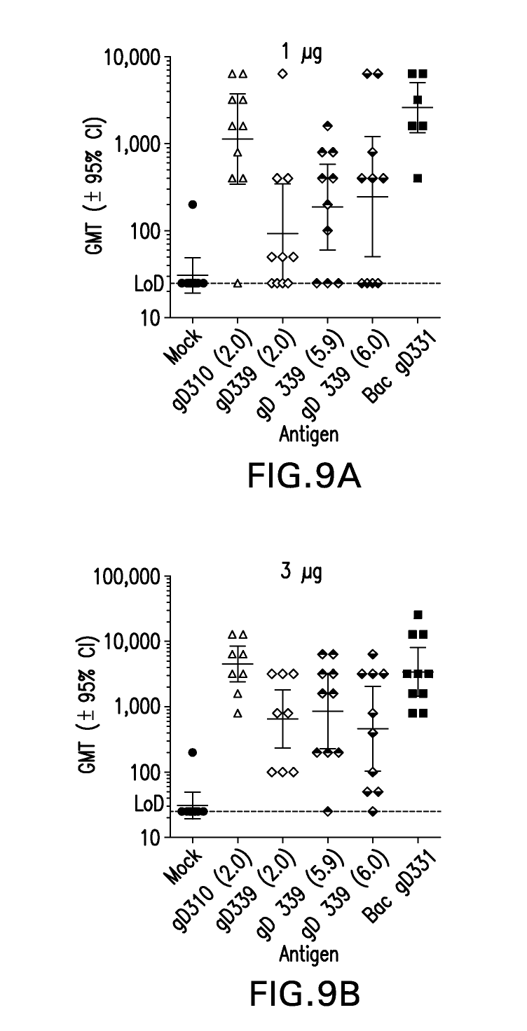

Mice vaccinated with 1 or 3 μg of gDt-2 elicited antibody titers against HSV-2 with geomean titers significantly higher than Mock (P<0.025, Mann-Whitney test) (FIG. 9). Of the animals vaccinated with 1 μg of antigen (FIG. 9A), 4 of 10 animals from in the gD 339 GFI2.0 and 6.0 groups, 3 of 10 from the gD 339 GFI5.9 group, and 1 of 10 animals from the gD 310 GFI2.0 group did not have antibodies reactive to HSV-2. All animals in the Bac 331 group elicited anti-HSV antibodies. Among the Pichia gD groups, antibody titers were significantly higher in animals receiving 1 μg gD 310 GFI2.0 than in animals receiving gD 339 GFI2.0 and gD 339 GFI5.9 (P=0.02), but not 339 GFI6.0 (P=0.12). Compared to 1 μg of Bac 331, all three gD 339 proteins elicited significantly less antibodies (P<0.01 for GFI 2.0 and 5.9 proteins and P=0.02 for GFI6.0 protein). There was no significant difference between Bac 331 and gD 310 GFI2.0 (P=0.26). In animals vaccinated with 3 μg of antigen (FIG. 9B), only 1 of 10 animals in the gD 339 GFI5.9 and 6.0 groups did not seroconvert. Animals receiving 3 μg of gD 310 GFI2.0 had significantly higher antibody titers to HSV-2 than animals receiving 3 μg of gD 339 (P<0.01 for GFI2.0 and 6.0 and P=0.03 for GFI5.9). Levels of antibodies were significantly higher in animals receiving 3 μg Bac 331 than in animals receiving gD 339 GFI2.0 and 6.0 (P=0.02 and 0.05, respectively). There were no significant differences in levels of antibodies among Bac 331, gD 310 GFI2.0 and gD 339 GFI5.9 (P=0.51 and 0.16, respectively).

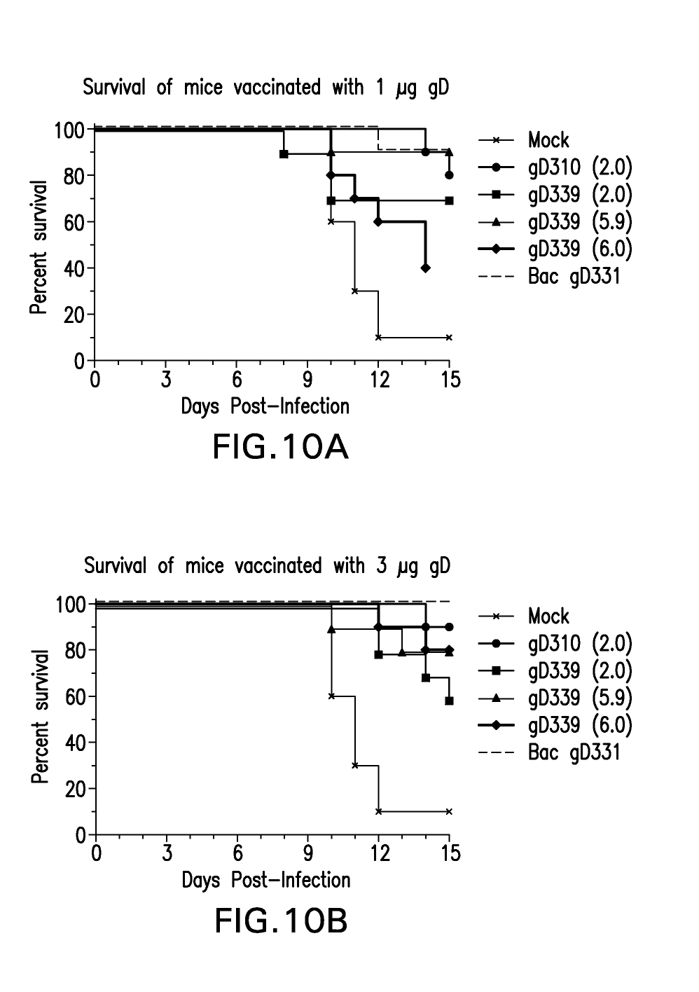

To test for the production of functional antibodies against HSV-2, mice were challenged intravaginally with HSV-2 strain G (FIG. 10). All 1 μg and 3 μg gDt-2 animal groups were significantly protected from vaginal HSV-2 challenge compared to Mock-vaccinated animals (P≦0.05, Log-rank test). In challenged mice, gD Bac 331, gD 310 GFI2.0 and gD 339 GFI5.9-vaccinated animals had the highest survival rates regardless of antigen concentration. Of mice vaccinated with 1 μg of gDt-2, Pichia gD 310 GFI2.0, gD 339 GFI5.9 and Bac 331 groups were significantly protected compared to mice vaccinated with gD 339 GFI6.0 (P=0.04, 0.03, and 0.03, respectively). Mice receiving 3 μg of gDt-2 showed significant differences in protection only between gD Bac 331 and gD 339 GFI2.0 (P=0.03).

### Example 5

### Guinea Pig HSV-2 Challenge Study Evaluating Pichia Expressed gD, gC and the Combination of gD+gC

Thirty-five female guinea pigs approximately 200-300 g were divided into seven groups of five. Vaccines were formulated utilizing Pichia pastoris (GFI2.0) expressed gC and gD 310 in the microgram doses listed in Table 4 in combination with Merck's amorphous aluminum hydroxyphosphate sulfate adjuvant and Iscomatrix.

TABLE 4
Vaccine groups

| Gp # | # of G. pigs | Antigen | Source | Dose | Adjuvant |
|---|---|---|---|---|---|
| 1 | 5 | gC | GFI2.0 | 10 | ISCOM + MAA |
| 2 | 5 | gC | GFI2.0 | 50 | ISCOM + MAA |
| 3 | 5 | gD 310 | GFI2.0 | 5 | ISCOM + MAA |
| 4 | 5 | gD 310 + gC | GFI2.0 | 5 + 10 | ISCOM + MAA |
| 5 | 5 | gD 310 + gC | GFI2.0 | 5 + 50 | ISCOM + MAA |
| 6 | 5 | gD 306 | CHO | 5 | MPL + AlOH |
| 7 | 5 | Mock | — | — | ISCOM + MAA |

The animals were immunized intramuscularly (IM) on days 0, 7 and 21 with bilateral hind flank injections (100 μl/injection for a total dose volume of 200 μl for groups 1-5 and 7, and a total dose volume of 125 μl for group 6). Sera were collected on Day 42, three weeks post-dose 3. On day 43, guinea pigs were anesthetized, swabbed vaginally three times to clear the vaginal tract and challenged with 1×10⁶, FU MS strain HSV-2. Vaginal swabs and disease score observations were collected daily for two weeks through the acute phase (Day 44 through Day 58) and disease score observations continued one additional week (through Day 65). Lumbosacral dorsal root ganglia (DRG) were extracted from challenge survivors on Day 72 for real-time PCR analysis.

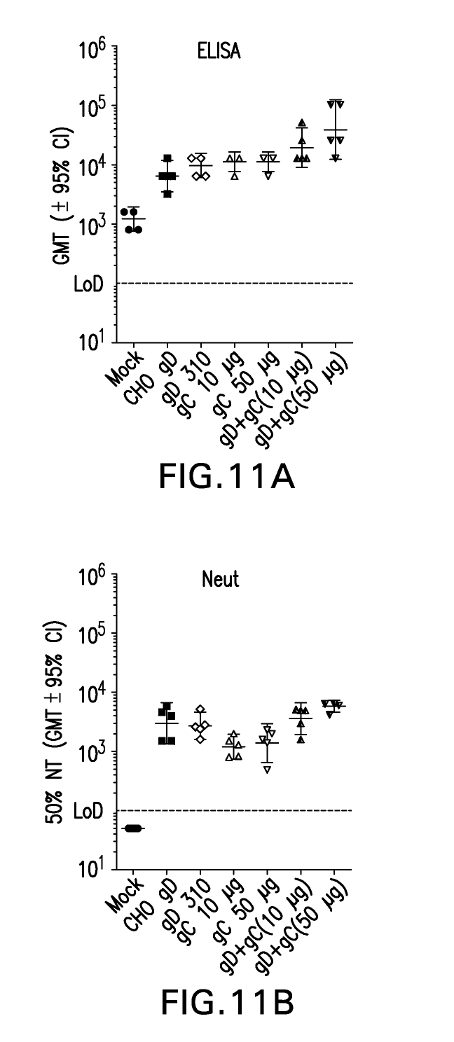

To evaluate the levels of total IgG produced against HSV-2,96-well Immulon 413 microtiter plates were coated overnight at 4° C. with 100 ng/well of purified HSV-2 lysate in bicarbonate buffer. The plates were blocked the following day and serial dilutions of guinea pig sera added to the wells. The plates were washed to remove any unbound antibody and bound IgG was detected at an OD of 405 nm using horseradish peroxidase-conjugated secondary antibody. ELISA geometric mean endpoint titers to HSV-2 lysate were plotted as the reciprocal dilution of the endpoint (FIG. 11A).

All vaccines elicited circulating IgG titers to HSV-2 that were significantly higher than Mock. The greatest antibody titers against HSV-2 lysates were achieved with the combination gD+gC vaccines (FIG. 11A).

Neutralization titers were evaluated using ELVIS® cells (Diagnostic HYBRIDS), which are a baby hamster kidney (BHK) cell line stably transfected with lacZ driven by the HSV-1 UL39 promoter. Upon HSV infection of ELVIS® cells, viral immediate-early genes induce the UL39-LacZ cassette. Guinea pig sera was diluted in culture media containing ˜6.7% rabbit complement and co-incubated with 1,800 PFU of HSV-2 strain MS for 1 h at 37° C. Antibody/virus mix was subsequently added to the ELVIS® cell monolayer and cultured overnight. LacZ expression results in β-galactosidase production. β-galactosidase activity was measured using a Gal-Screen kit (Applied Biosystems). The 50% neutralization titer represents the dilution of serum at which β-galactosidase activity levels are 50% that of the positive control (HSV-2 in the absence of serum). The geometric mean 50% neutralization titers are plotted as the reciprocal dilution (FIG. 11B)

The results showed that all vaccines demonstrated HSV-2 neutralization potential. The Mock vaccinated animals did not (FIG. 11B). The vaccines containing any combination of gD had greater neutralization potential than the gC alone vaccines.

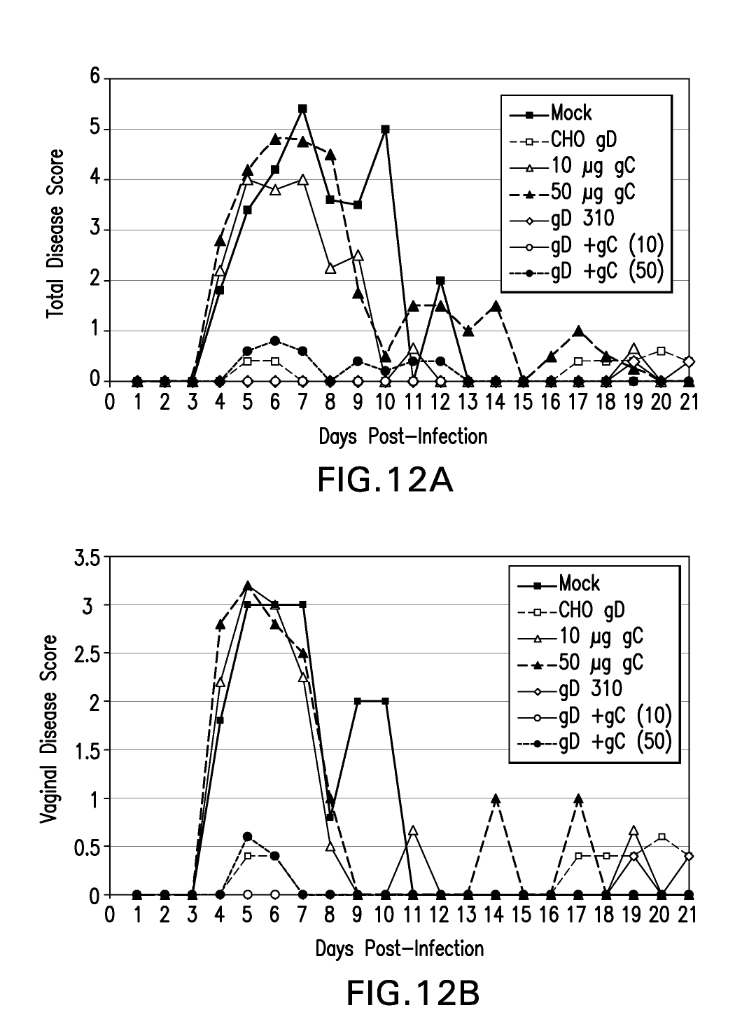

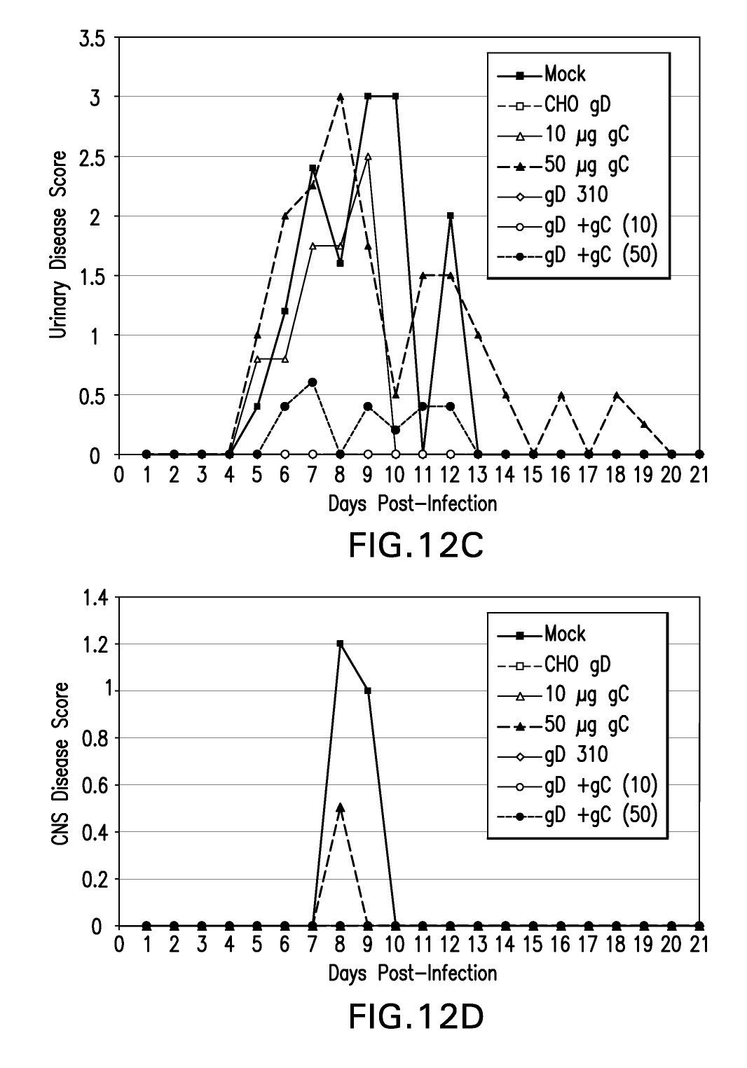

To evaluate the extent of HSV-2 vaginal disease, a scoring system of infection site disease was adapted from Stanberry et al., 1982 (J. Infect Dis, 146:397-404), whereby infected animals were given a score of 0 to 4 (FIG. 12B). Guinea pigs given a score of 0 exhibited no signs of HSV infection. A score of 1 denoted redness and/or swelling of the perineum. Animals with a score of 2 exhibited 1 or 2 herpetic lesions or had 1 or more healing lesions on the perineum whereas those with a score of 3 had 3 or more lesions. Guinea pigs that had a score of 4 exhibited coalesced or ulcerated lesions on the perineum. Urinary tract disease was also scored (FIG. 12C). A score of 1 denoted excretion of apparent purulent exudate during urination. A score of 2 was given to animals with urinary retention. Guinea pigs with a score of 3 expressed bloody urine. Notably, severe hemorrhagic cystitis was the primary cause of removal of animals from the study. Central nervous disease scores (FIG. 12D) ranged from 1 to 4 where 1 denoted ruffled fur and/or depression, 2 denoted weak hind legs, 3 denoted unilateral hind leg paralysis and 4 denoted bilateral hind leg paralysis. The total disease score (FIG. 12A) reflects the sum of the infection site disease, urinary system, and central nervous disease scores. The results of the total diseae score and the individual disease scores for each vaccine group are plotted as the mean+standard error of the mean (FIG. 12).

Mock-vaccinated animals exhibited severe infection site and urinary system disease. During the acute phase of infection, these animals presented with edema, erythema, and herpetic lesions on the perineum between days ˜3 and 11 post infection (pi) and purulent exudate and blood containing urine-containing urine and acute urinary retention between days ˜7 and 14 pi. The gD+gC(10) had no notable disease (FIG. 12). This group demonstrated significantly reduced total disease throughout the 21 day observation period compared to all groups except Pichia expressed gD alone. Picha gD 310 had no acute disease and minor disease during the reactivation period at days 19 and 21.

The overall number of lesions was reduced substantially in all gDt-2 vaccinated groups relative to Mock (data not shown). Vaccination with Pichia gCt-2 reduced the total number of lesions by 2.4 to 2.6-fold relative to Mock. Immunization with any gD or gD+gC vaccine reduced the number of lesions by greater than 40 to 120 fold.

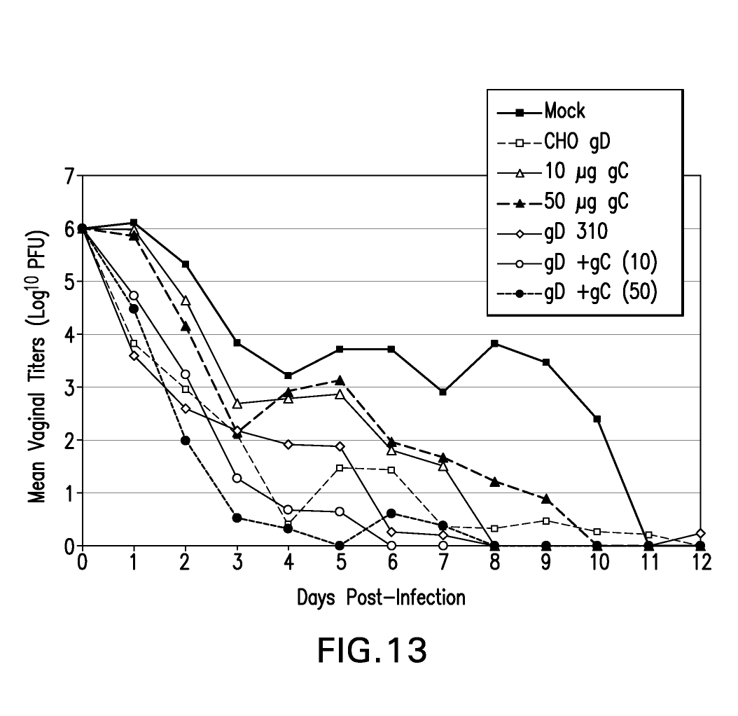

To evaluate the levels of HSV-2 shed into the vaginal vault, the vaginas of guinea pigs were swabbed daily during the acute phase of viral replication. Vaginal swab samples were frozen at −80° C. in tissue culture medium. Frozen samples were later thawed, thoroughly mixed, and infectious virus quantified by standard plaque assays on Vero cell monolayers. Viral titers per swab were determined and the results of each vaccine group are plotted as the mean+standard error of the mean (FIG. 13).

In Mock animals, HSV-2 titers peaked on day 1-2 and were not detected by day 11 p.i. Animals vaccinated with gD+gC(10) had the shortest number of days of detectable vaginal titers and virus was not detectable by day 6. In all vaccinated animals, the levels of virus in vaginal swabs were significantly lower than Mock (FIG. 13). gD 310, gD+gC(50) and gC(10) had no detectable titers by day 8. gD CHO titers declined at a slower rate compared to gD 310 or gD+gC vaccines and had low detectable titers to day 12. Notably, gD+gC(10) exhibited no disease throughout the observation period (FIG. 12) and rapid reduction in vaginal titers, to below the level of detection, showing a correlation between viral loads in the vagina and the severity of disease.

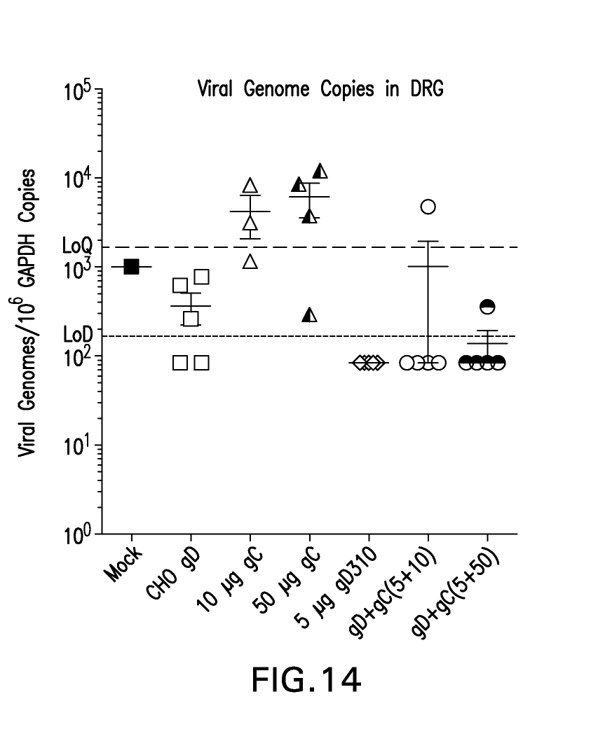

To determine the efficiency of the establishment of latency in vaccinated animals, surviving guinea pigs were euthanized and lumbosacral DRG were harvested. Total DNA was isolated from DRG using the Qiamp DNA mini kit (Qiagen, Hilden, Germany) as per the manufacturer's instructions. HSV-2 DNA was quantified in duplicate using a primer/probe set to the gG gene (US4) and normalized to the host cell gene, GAPDH. Quadruplet standard curves were constructed from 10-fold dilutions of gG-2 plasmid and purified guinea pig DNA (BioChain Institute, Inc.). The DNA sample along with oligonucleotide primer/probe sets specific for gG-2 and GAPDH were mixed with Quantitect Multiplex PCR no Rox Master Mix (Qiagen). Reactions were carried out in a Stratagene Mx3005P Real Time PCR system and analyzed with Stratagene MxPro Software. The results are plotted as the copy number of HSV-2 DNA per 10⁶ copies of GAPDH (FIG. 14). Notably, samples that had no detectable viral genomes were given a value that was 50% of the LoD (168 genome copies).

The viral genomes in the DRG were substantially reduced in the gD310 and gD+gC vaccine groups. Viral genome titers below the level of quantitation cannot be accurately calculated and viral genomes below the level of detection cannot be accurately assessed in the assay. Thus GMTs calculated with values falling in these ranges should be considered with those reservations in mind. Clearly the DRG viral loads were markedly reduced in all animals receiving any gD (FIG. 14). The correlation between reduction in disease, reduced vaginal viral titers and reduced viral genomes in the DRG appears to hold for the Pichia expressed gD and gD+gC vaccines.

In summary, all vaccines tested (gD, gC and gD+gC) elicited high HSV-2-specific antibody titers with neutralizing activity. The combination vaccine produced the highest titers to HSV2 lysates; however, the gD and gD+gC vaccines had overlapping neutralizing titers confidence intervals. Vaccination with gD+gC and gD alone vaccines produced significant reduction in total disease and reduced the frequency and the total number of lesions exhibited during HSV-2 infection. gD+gC(10) animals demonstrated complete protection from disease. Vaccination with gC alone reduced the total number of lesions but did not show significant differences in total disease score or cumulative lesion days when compared to Mock. The gD+gC and gD alone vaccines can significantly reduce the levels of virus shed in guinea pig vaginas. gD+gC and gD alone vaccination substantially reduced the viral genome loads in lumbosacral DRG and thus most effectively reduced or prevented the establishment of viral latency in the neurons. The gD+gC(10) vaccine demonstrated the most favorable and the gD Pichia vaccine showed the next best efficacy in protecting guinea pigs from HSV-2 disease and in addition, positively reduced the burden of disease as assessed by viral vaginal titers and DRG viral genome loads.

What is claimed is:

1. A composition of a recombinant Herpes Simplex Virus glycoprotein selected from the group consisting of gC, gD, gB and gE, or an immunogenic fragment thereof, said composition comprising a plurality of bi-antennary N-linked glycans attached to the glycoprotein, said plurality of N-linked glycans comprising greater than 50 mole percent of an N-linked glycan consisting essentially of a structure selected from the group consisting of Man₅GlcNAc₂; NANA₂Gal₂GlcNAc₂Man₃GlcNAc₂; GlcNAc₂Man₃GlcNAc₂; Man₃GlcNAC₂; NANAGalGlcNAcMan₅GlcNAc₂; Gal GlcNAc Man₅GlcNAc₂; GlcNAcMan₅GlcNAc₂; and Man₈GlcNAc₂.

2. The composition of claim 1, wherein said glycoprotein protein is gC, gD, or an immunogenic fragment thereof.

3. The composition of claim 2, wherein said glycoprotein is a HSV-1 glycoprotein.

4. The composition of claim 2, wherein said glycoprotein is a HSV-2 glycoprotein.

5. The composition of claim 1, wherein said plurality of N-linked glycans comprises greater than 50 mole percent of an N-linked glycan consisting essentially of a structure selected from the group consisting of Man₅GlcNAc₂; and NANA₂Gal₂GlcNAc₂Man₃GlcNAc₂.

6. The composition of claim 1, wherein said plurality of N-linked glycans comprises greater than 75 mole percent of an N-linked glycan consisting essentially of Man₅GlcNAc₂.

7. The composition of claim 1, wherein said plurality of N-linked glycans comprises greater than 85 mole percent of an N-linked glycan consisting essentially of Man₅GlcNAc₂.

8. The composition of claim 1, wherein said plurality of N-linked glycans comprises greater than 55 mole percent of an N-linked glycan consisting essentially of NANA₂Gal₂GlcNAc₂Man₃GlcNAc₂.

9. The composition of claim 1, wherein said plurality of N-linked glycans comprises greater than 70 mole percent of an N-linked glycan consisting essentially of NANA₂Gal₂GlcNAc₂Man₃GlcNAc₂.

10. An immunogenic composition comprising:
a) a composition of claim 1, wherein said glycoprotein is gD protein or immunogenic fragment thereof;
b) a recombinant HSV gC protein or immunogenic fragment thereof; and
c) an adjuvant.

11. The vaccine of claim 10, wherein said gD protein and said gC protein are HSV-2 proteins.

12. The vaccine of claim 10, further comprising one or more recombinant HSV-2 protein selected from a gB protein and a gE protein.

13. The vaccine of claim 10, wherein said adjuvant is a CpG-containing nucleotide molecule, an aluminum salt adjuvant, a saponin-based adjuvant, or any combination thereof.

14. The vaccine of claim 11, wherein the adjuvant is a combination of an aluminum salt adjuvant and a saponin-based adjuvant.

15. An immunogenic composition comprising:
a) a recombinant HSV gD protein or immunogenic fragment thereof;
b) a composition of claim 1, wherein said glycoprotein is gC protein or immunogenic fragment thereof; and;
c) an adjuvant.

16. The vaccine of claim 15, wherein said gD protein and said gC protein are HSV-2 proteins.

17. The vaccine of claim 15, further comprising one or more recombinant HSV-2 protein selected from a gB protein and a gE protein.

18. The vaccine of claim 15, wherein said adjuvant is a CpG-containing nucleotide molecule, an aluminum salt adjuvant, a saponin-based adjuvant, or any combination thereof.

19. The vaccine of claim 18, wherein the adjuvant is a combination of an aluminum salt adjuvant and a saponin-based adjuvant.

20. An immunogenic composition comprising:
a) a composition of claim 1, wherein said glycoprotein is gD protein or immunogenic fragment thereof;
b) a composition of claim 1, wherein said glycoprotein is gC protein or immunogenic fragment thereof; and;
c) an adjuvant.

21. The vaccine of claim 20, wherein said gD protein and said gC protein are HSV-2 proteins.

22. The vaccine of claim 20, further comprising one or more recombinant HSV-2 protein selected from a gB protein and a gE protein.

23. The vaccine of claim 20, wherein said adjuvant is a CpG-containing nucleotide molecule, an aluminum salt adjuvant, a saponin-based adjuvant, or any combination thereof.

24. The vaccine of claim 23, wherein the adjuvant is a combination of an aluminum salt adjuvant and a saponin-based adjuvant.

25. A method for the production of recombinant Herpes Simplex Virus glycoprotein gC, gB or gE comprising:
a) transforming Pichia pastoris with an expression vector containing a DNA encoding a HSV protein selected from the group consisting of gC, gB and gE, which is under regulation of a promoter functional in Pichia pastoris;
b) culturing said Pichia pastoris in a medium; and
c) recovering/isolating the HSV protein from the cultured Pichia pastoris,
wherein said Pichia pastoris is a strain in which one or more of the following modifications have been made: i) disruption of the yeast protein disulfide isomerase gene (PDT); or ii) expression of one or more chaperones selected from the group consisting of human PDI, human calreticulin (hCRT), human Erp57, human FLC1, human ERO1, human FAD1.

26. The method of claim 25, wherein said Pichia pastoris is a glycoengineered Pichia pastoris strain selected from 2.0, 5.9, and 6.0.

27. An isolated nucleic acid comprising a nucleotide sequence of SEQ ID NO: 10, 12, 14, or 16.

28. The isolated nucleic acid of claim 27, further comprising a signal sequence or HIS tag.
